(19) United States
(12) Patent Application Publication
(10) Pub. No.: US 2026/0137665 A1
(43) Pub. Date: May 21, 2026

(54) COMBINATION THERAPIES FOR THE TREATMENT OF CANCER

(71) Applicant: AADI Bioscience, Inc., Pacific Palisades (CA)

(72) Inventors: Shihe HOU, Millington, NJ (US); Andrew KWON, Manhattan Beach, CA (US); Neil P. DESAI, Pacific Palisades, CA (US)

(21) Appl. No.: 19/119,692

(22) PCT Filed: Oct. 10, 2023
(86) PCT No.: PCT/US2023/076507

§ 371 Date: Apr. 9, 2025

Related U.S. Application Data
(60) Provisional application No. 63/415,252, filed on Oct. 11, 2022.
(60) Provisional application No. 63/461,145, filed on Apr. 21, 2023.

(51) Int. Cl. A61K 31/436; A61K 9/51; A61K 31/416; A61K 31/496; A61K 31/4985; A61K 31/517; A61K 31/519; A61K 31/5386; A61P 35/00

(52) U.S. Cl. CPC A61K 31/436; A61K 9/5169; A61K 31/416; A61K 31/496; A61K 31/4985; A61K 31/517; A61K 31/519; A61K 31/5386; A61P 35/00

(57) ABSTRACT

Provided are methods of treating cancer (e.g, a cancer that comprises one or more cancer cells that express a KRAS G12C mutant protein and/or have at least one mTOR-activating aberration) in an individual that comprise administering a composition comprising nanoparticles that comprise an mTOR inhibitor (such as a limus drug, e.g, sirolimus or a derivative thereof) and an albumin in combination with a KRAS G12C inhibitor to the individual. Also provided are related kits.

### CROSS-REFERENCE TO RELATED APPLICATIONS

This application claims the priority benefit of U.S. Patent Application Ser. No. 63/415,252, filed Oct. 11, 2022 and U.S. Patent Application Ser. No. 63/461,145 filed Apr. 21, 2023, the entire contents of which are incorporated herein by reference for all purposes.

### FIELD OF THE INVENTION

The present application relates to combination therapies for the treatment of cancer, e.g., cancers characterized by expression of a KRAS mutant protein (e.g., a KRAS G12C mutant protein) and by dysregulated (e.g., activated) mTOR signaling.

### BACKGROUND

The mammalian target of rapamycin (mTOR) is a conserved serine/threonine kinase that serves as a central hub of signaling in the cell to integrate intracellular and extracellular signals and to regulate cellular growth and homeostasis. Activation of the mTOR pathway is associated with cell proliferation and survival, while inhibition of mTOR signaling leads to inflammation and cell death. Dysregulation of the mTOR signaling pathway has been implicated in an increasing number of human diseases, including cancer and autoimmune disorders. Consequently, mTOR inhibitors have found wide applications in treating diverse pathological conditions such as solid tumors, hematological malignancies, organ transplantation, restenosis, and rheumatoid arthritis.

Sirolimus (INN/USAN), also known as rapamycin, is an immunosuppressant drug used to prevent rejection in organ transplantation; it is especially useful in kidney transplants. Sirolimus-eluting stents were approved in the United States to treat coronary restenosis. Additionally, sirolimus has been demonstrated as an effective inhibitor of tumor growth in various cell lines and animal models. Other limus drugs, such as analogs of sirolimus, have been designed to improve the pharmacokinetic and pharmacodynamic properties of sirolimus. For example, Temsirolimus was approved in the United States and Europe for the treatment of renal cell carcinoma. Everolimus was approved in the U.S. for treatment of advanced breast cancer, pancreatic neuroendocrine tumors, advanced renal cell carcinoma, and subependymal giant cell astrocytoma (SEGA) associated with Tuberous Sclerosis. The mode of action of sirolimus is to bind the cytosolic protein FK-binding protein 12 (FKBP12), and the sirolimus-FKBP12 complex in turn inhibits the mTOR pathway by directly binding to the mTOR Complex 1 (mTORC1).

Albumin-based nanoparticle compositions have been developed as a drug delivery system for delivering substantially water insoluble drugs. See, for example, U.S. Pat. Nos. 5,916,596; 6,506,405; 6,749,868, and 6,537,579, 7,820,788, and 7,923,536. Abraxane®, an albumin stabilized nanoparticle composition containing paclitaxel, was approved in the United States in 2005 and subsequently in various other countries for treating metastatic breast cancer, pancreatic cancer, and non-small cell lung cancer. FYARRO®, an albumin stabilized nanoparticle composition containing sirolimus, was recently approved for treating PEcoma.

KRAS mutations also play a role in some of the most common and deadly cancers, including lung, colon, colorectal, and rectal cancers. KRAS mutations are estimated to be present in approximately 25% of tumors. One single type of KRAS mutation, i.e., the KRAS G12C mutation, accounts for about 44% of all KRAS mutations. G12C is a single point mutation with a glycine-to-cysteine substitution at codon 12 of the KRAS protein. This substitution favors the active, GTP-bound conformation of KRAS, amplifying signaling pathways that lead to oncogenesis. KRAS G12C is particularly prevalent in non-small cell lung cancer (NSCLC), which makes up about 85% of all lung cancer cases in the U.S. Approximately 13% of Americans with NSCLC have the KRAS G12C mutation, and there are about 23,000 new cases of KRAS G12C NSCLC diagnosed every year in the U.S. alone. Clinical trials of KRAS G12C allele-specific inhibitors adagrasib and sotorasib have shown promising activity in cancers expressing the KRAS G12C mutant protein. However, the clinical trial data also indicates that there is significant variation in response among patients treated with KRAS G12C inhibitors and that KRAS G12C inhibitor monotherapy is unlikely to be sufficient to elicit a sustained therapeutic response.

Accordingly, there is a continuing need in the art for methods of treating cancers that exhibit dysregulation of the mTOR signaling pathway and express the KRAS G12C mutant protein.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In some embodiments, provided is a method of treating cancer in an individual, comprising administering to the individual: (a) an effective amount of a composition comprising nanoparticles comprising an mTOR inhibitor and an albumin, and (b) an effective amount of a KRAS inhibitor. In some embodiments, the cancer comprises one or more cancer cells that express a KRAS mutant protein. In some embodiments, the cancer comprises (such as further comprises) one or more cancer cells that have at least one mTOR-activating aberration. In some embodiments, the individual is human.

In some embodiments, the mTOR inhibitor is a limus drug. In some embodiments, the limus drug is sirolimus. In some embodiments, the average diameter of the nanoparticles in the composition is no greater than about 150 nm. In some embodiments, the average diameter of the nanoparticles in the composition is no greater than about 120 nm. In some embodiments, the weight ratio of the albumin to the mTOR inhibitor in the nanoparticle composition is no greater than about 10:1. In some embodiments, the nanoparticles comprise the mTOR inhibitor associated with the albumin. In some embodiments, the nanoparticles comprise the mTOR inhibitor coated with the albumin. In some embodiments, the mTOR inhibitor nanoparticle composition is administered intravenously or subcutaneously. In some embodiments, the mTOR inhibitor nanoparticle composition is administered intravenously.

In some embodiments, the KRAS inhibitor is an antibody, a peptide, a protein, an antisense oligonucleotide, or a small molecule that inhibits the activity of the KRAS mutant protein. In some embodiments, the KRAS inhibitor is a small molecule. In some embodiments, the KRAS inhibitor is a small molecule KRAS G12C inhibitor selected from the group consisting of: sotorasib, adagrasib, JAB-21822, GDC-6036, JDQ443, D-1553, GH35, GFH925, BPI-421286, and LY3537982, RMC-6291, RMC-8839, HBI-2438, and JNJ-74699157. In some embodiments, the KRAS G12C inhibitor small molecule is sotorasib or adagrasib. In some embodiments, the sotorasib or the adagrasib is administered orally. In some embodiments, the cancer comprises one or more cancer cells that express a KRAS G12C mutant protein. In some embodiments, the KRAS inhibitor is a small molecule KRAS G12D inhibitor selected from the group consisting of: MRTX1133 and RMC-6236. In some embodiments, the cancer comprises one or more cancer cells that express a KRAS G12D mutant protein. In some embodiments, the KRAS inhibitor is a small molecule KRAS G12V inhibitor, and wherein the small molecule KRAS G12V inhibitor is JAB-23000. In some embodiments, the cancer comprises one or more cancer cells that express a KRAS G12V mutant protein.

In some embodiments, the cancer that comprises one or more cancer cells that express a KRAS mutant protein and/or have at least one mTOR-activating aberration is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin. In some embodiments, the cancer is solid tumor, lung cancer, bladder cancer, appendiceal cancer, colorectal cancer, small bowel cancer, pancreatic cancer, or tumor of unknown origin. In some embodiments, the cancer or tumor (such as any of the preceding cancers or tumors) is advanced, unresectable, and/or metastatic. In some embodiments, the cancer is solid tumor (e.g., advanced, unresectable, and/or metastatic solid tumor), lung cancer (e.g., advanced, unresectable, and/or metastatic lung cancer), or bladder cancer (e.g., advanced, unresectable, and/or metastatic bladder cancer). In some embodiments, the lung cancer is non-small cell lung cancer (NSCLC), e.g., advanced, unresectable, and/or metastatic NSCLC.

In some embodiments, the mTOR inhibitor nanoparticle composition and the KRAS inhibitor are administered simultaneously. In some embodiments, the mTOR inhibitor nanoparticle composition and the KRAS inhibitor are administered concurrently. In some embodiments, the mTOR inhibitor nanoparticle composition and the KRAS inhibitor are administered sequentially. In some embodiments, the mTOR inhibitor nanoparticle composition is administered weekly, once every three weeks, or twice every three weeks. In some embodiments, the KRAS inhibitor is administered daily or twice every day.

In some embodiments, the method comprises selecting the individual for treatment based on the presence of one or more cancer cells with at least one mTOR-activating aberration prior to the administration of the mTOR inhibitor nanoparticle composition and the KRAS inhibitor. In some embodiments, the mTOR-activating aberration comprises a mutation in an mTOR-associated gene. In some embodiments, the mTOR-activating aberration is in at least one mTOR-associated gene selected from the group consisting of: AKT1, FLT-3, MTOR, PIK3CA, PIK3CG, TSC1, TSC2, RHEB, STK11, NF1, NF2, TP53, FGFR4, BAP1, KRAS, NRAS, NRF2, KEAP1, and PTEN. In some embodiments, the mTOR activating aberration is in TSC1 and/or TSC2. In some embodiments, the method comprises (such as further comprises) selecting the individual for treatment based on the presence of one or more cancer cells that express a KRAS mutant protein. In some embodiments, the KRAS mutant protein is a KRAS G12C mutant protein, a KRAS G12D mutant protein, or a KRAS G12V mutant protein.

In some embodiments, provided is a kit for treating cancer in a subject comprising: (a) a composition comprising nanoparticles comprising an mTOR inhibitor and an albumin, and (b) instructions for administering an effective amount of the mTOR inhibitor nanoparticle composition and an effective amount of a KRAS inhibitor to a subject who has a cancer that comprises one or more cancer cells that express a KRAS mutant protein and/or have at least one mTOR-activating aberration. In some embodiments, the cancer is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin. In some embodiments, the individual is human.

In some embodiments, the mTOR inhibitor is a limus drug. In some embodiments, the limus drug is sirolimus. In some embodiments, the average diameter of the nanoparticles in the composition is no greater than about 150 nm. In some embodiments, the average diameter of the nanoparticles in the composition is no greater than about 120 nm. In some embodiments, the weight ratio of the albumin to the mTOR inhibitor in the nanoparticle composition is no greater than about 10:1. In some embodiments, the nanoparticles comprise the mTOR inhibitor associated with the albumin. In some embodiments, the nanoparticles comprise the mTOR inhibitor coated with the albumin.

In some embodiments, the KRAS inhibitor is an antibody, a peptide, a protein, an antisense oligonucleotide, or a small molecule that inhibits the activity of the KRAS mutant protein. In some embodiments, the KRAS inhibitor is a small molecule. In some embodiments, the KRAS inhibitor is a small molecule KRAS G12C inhibitor selected from the group consisting of: sotorasib, adagrasib, JAB-21822, GDC-6036, JDQ443, D-1553, GH35, GFH925, BPI-421286, and LY3537982, RMC-6291, RMC-8839, HBI-2438, and JNJ-74699157. In some embodiments, the KRAS inhibitor is a small molecule KRAS G12D inhibitor selected from the group consisting of: MRTX1133 and RMC-6236. In some embodiments, the KRAS inhibitor is a small molecule KRAS G12V inhibitor, and wherein the small molecule KRAS G12V inhibitor is JAB-23000.

### BRIEF DESCRIPTION OF THE DRAWINGS

FIG. 1A provides the results of experiments that were performed to assess the anti-tumor activity of (i) nab-sirolimus, (ii) everolimus, (iii) sotorasib, (iv) nab-sirolimus+sotorasib, and (v) everolimus+sotorasib in mice bearing NCI-H2030 human non-small cell lung cancer xenografts.

FIG. 1B provides waterfall plots showing tumor volume regression in NCI-H2030-xeongrafted mice treated with saline, nab-sirolimus, everolimus, sotorasib, nab-sirolimus+sotorasib, and everolimus+sotorasib.

FIG. 1C shows the % change in body weight in NCI-H2030-xeongrafted mice treated with saline, nab-sirolimus, everolimus, sotorasib, nab-sirolimus+sotorasib, and everolimus+sotorasib.

FIG. 2A provides the results of experiments that were performed to assess the anti-tumor activity of (i) nab-sirolimus, (ii) everolimus, (iii) sotorasib, (iv) adagrasib, (v) nab-sirolimus+sotorasib, (vi) everolimus+sotorasib, (viii) nab-sirolimus+adagrasib, and (viii) everolimus+adagrasib in mice bearing NCI-H2122 human non-small cell lung cancer xenografts.

FIG. 2B provides waterfall plots showing tumor volume regression in NCI-H2122-xeongrafted mice treated with saline, nab-sirolimus, everolimus, sotorasib, adagrasib, nab-sirolimus+sotorasib, everolimus+sotorasib, nab-sirolimus+adagrasib, and everolimus+adagrasib.

FIG. 2C shows the % change in body weight in NCI-H2122-xeongrafted mice treated with saline, nab-sirolimus, everolimus, sotorasib, adagrasib, nab-sirolimus+sotorasib, everolimus+sotorasib, nab-sirolimus+adagrasib, and everolimus+adagrasib.

FIG. 3A provides the results of experiments that were performed to assess the anti-tumor activity of (i) nab-sirolimus, (ii) sotorasib, (iii) adagrasib, (iv) nab-sirolimus+sotorasib, and (v) nab-sirolimus+adagrasib in mice bearing UMUC3 human bladder cancer xenografts.

FIG. 3B provides waterfall plots showing tumor volume regression in UMUC3-xeongrafted mice treated with saline, nab-sirolimus, sotorasib, adagrasib, nab-sirolimus+sotorasib, and nab-sirolimus+adagrasib.

FIG. 3C shows the % change in body weight in UMUC3-xeongrafted mice treated with saline, nab-sirolimus, sotorasib, adagrasib, nab-sirolimus+sotorasib, and nab-sirolimus+adagrasib.

FIG. 4 shows the anti-tumor activity of single agent sotorasib and single agent adagrasib in mice bearing NCI-H2122 NSCLC tumors (left side, data taken from FIG. 2A), as well as the anti-tumor activity of single agent sotorasib and single agent adagrasib in mice bearing UMUC3 tumors (right side, data taken from FIG. 3A).

FIG. 5 shows the study designs of the Phase 1 and Phase 2 portions of the clinical trial described in Example 4.

FIG. 6 shows comparison of nab-sirolimus and everolimus trough (A) tumor and (B) blood concentrations in NSCLC (adenocarcinoma) NCI-H2122 model described in Example 1.

FIGS. 7A-7B show western blot results of phospho-S6 and phosphor-4EBP1 in tumor cells after treatment with nab-sirolimus, everolimus, or a combination of nab-sirolimus/everolimus and one of sotorasib or adagrasib.

### DETAILED DESCRIPTION

### Overview

The present application is based on the unexpected finding that a combination treatment comprising a composition comprising nanoparticles comprising an mTOR inhibitor and an albumin (e.g., an “mTOR inhibitor nanoparticle composition,” such as a sirolimus/albumin nanoparticle composition) and a KRAS inhibitor (e.g., a KRAS G12C inhibitor such as sotorasib or adagrasib) is significantly more effective in inhibiting the growth of tumors than either agent alone. Applicant also found that such combination treatment was more effective in inhibiting tumor growth than a combination treatment comprising a non-nanoparticle mTOR inhibitor (e.g., everolimus) and a KRAS inhibitor (e.g., a KRAS G12C inhibitor such as sotorasib or adagrasib). The improved tumor growth inhibition (TGI) of the combination treatment comprising an mTOR inhibitor nanoparticle composition and a KRAS inhibitor (e.g., a KRAS G12C inhibitor) correlated with significantly higher rate of tumor response (e.g., tumor regression of more than −30% change in tumor volume) than single agent treatment with an mTOR inhibitor nanoparticle composition, single agent treatment with a KRAS inhibitor (e.g., a KRAS G12C inhibitor), and combination treatment comprising a non-nanoparticle mTOR inhibitor and a KRAS G12C inhibitor (e.g., a KRAS G12C inhibitor).

The present application therefore in one aspect provides methods of treating cancer in an individual that comprise administering to the individual (a) an effective amount of a composition comprising nanoparticles comprising an mTOR inhibitor and an albumin (e.g., nab-sirolimus) and (b) an effective amount of a KRAS inhibitor (e.g., a KRAS G12C inhibitor). In some embodiments, the cancer comprises one or more cells that express a KRAS mutant protein (e.g., a KRAS G12C mutant protein). The mTOR pathway is often activated in cancer patients with KRAS mutation and contributes to adaptive resistance to KRAS inhibitors (Byun et al. (2019) “Oncogenic KRAS signaling activates mTORC1 through COUP-TFII-mediated lactate production.” EMBO Rep 20(6).) Additionally or alternatively, in some embodiments, the tumor comprises cancer cell(s) that have at least one mTOR-activating aberration. In another aspect, provided are kits and articles of manufacture for the treatment of cancer, e.g., a cancer comprising one or more cells that express a KRAS mutant protein (e.g., a KRAS G12C mutant protein) and/or have at least one mTOR-activating aberration, which include a composition comprising nanoparticles comprising an mTOR inhibitor and an albumin (e.g., nab-sirolimus). In some embodiments, the kits and articles of manufacture comprise instructions for administering the composition comprising the mTOR inhibitor and an albumin to an individual in combination with a KRAS inhibitor (e.g., a KRAS G12C inhibitor) to treat cancer, e.g., a cancer comprising one or more cells that express the KRAS mutant protein (e.g., a KRAS G12C mutant protein) and/or have at least one mTOR-activating aberration. In some embodiments, the kits and articles of manufacture further comprise an KRAS inhibitor (e.g., a KRAS G12C inhibitor).

### Definitions

Before describing the embodiments in detail, it is to be understood that the present disclosure is not limited to particular compositions or biological systems, which can, of course, vary. It is also to be understood that the terminology used herein is for the purpose of describing particular embodiments only and is not intended to be limiting.

As used herein “nab” stands for nanoparticle albumin-bound, and “nab-sirolimus” is an albumin stabilized nanoparticle formulation of sirolimus (rapamycin). nab-sirolimus is also known as nab-rapamycin, which has been previously described. See, for example, WO2008109163A1, WO2014151853, WO2008137148A2, and WO2012149451A1, each of which is incorporated herein by reference in their entirety.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequences of cancer. The methods described herein contemplate any one or more of these aspects of treatment.

As used herein, an “at risk” individual is an individual who is at risk of developing cancer. An individual “at risk” may or may not have detectable disease and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

The term “effective amount” used herein refers to an amount of a compound or composition sufficient to treat a specified disorder, condition, or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer.

As is understood in the art, an “effective amount” may be in one or more doses, i.e., a single dose or multiple doses may be required to achieve the desired treatment endpoint. An effective amount may be considered in the context of administering one or more therapeutic agents, and a nanoparticle composition (e.g., a composition including sirolimus and an albumin) may be considered to be given in an effective amount if, in conjunction with one or more other agents, a desirable or beneficial result may be or is achieved. The components (e.g., the first and second therapies) in a combination therapy described herein may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality, such as administration of a nanoparticle composition described herein in addition to administration of the other agent to the same individual under the same treatment plan. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, the term “subject” for purposes of treatment refers to any animal classified as a mammal, including humans, domestic and farm animals, and zoo, sports, or pet animals, such as dogs, horses, cats, cows, etc. Preferably, the mammal is human.

As used herein, “specific,” “specificity,” or “selective” or “selectivity” as used when describing a compound as an inhibitor, means that the compound preferably interacts with (e.g., binds to, modulates, and inhibits) a particular target (e.g., a protein and an enzyme) than a non-target. For example, the compound has a higher affinity, a higher avidity, a higher binding coefficient, or a lower dissociation coefficient for a particular target. The specificity or selectivity of a compound for a particular target can be measured, determined, or assessed by using various methods well known in the art. For example, the specificity or selectivity can be measured, determined, or assessed by measuring the IC50 of a compound for a target. A compound is specific or selective for a target when the IC50 of the compound for the target is 2-fold, 4-fold, 6-fold, 8-fold, 10-fold, 20-fold, 50-fold, 100-fold, 500-fold, 1000-fold, or more lower than the IC50 of the same compound for a non-target. For example, the IC50 of a KRAS G12C inhibitor is 2-fold, 4-fold, 6-fold, 8-fold, 10-fold, 20-fold, 50-fold, 100-fold, 500-fold, 1000-fold, or more lower than the IC50 of the same KRAS G12C inhibitor for wild type KRAS. IC50 can be determined by commonly known methods in the art.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/that or are included on the Inactive Ingredient Guide prepared by the U.S. Food and Drug administration.

It is understood aspects and embodiments of the present disclosure include “comprising,” “consisting,” and/or “consisting essentially of” aspects and embodiments.

Reference to “about” a value or parameter herein includes (and describes) variations that are directed to that value or parameter per se. For example, description referring to “about X” includes description of “X.”

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

### Methods of Treating Cancer

In some embodiments, provided is a method of treating a cancer in an individual (e.g., a human), comprising administering to the individual (a) an effective amount of a composition comprising nanoparticles comprising an mTOR inhibitor (such as a limus drug, e.g., sirolimus or a derivative or analog thereof) and an albumin (an “mTOR inhibitor nanoparticle composition”); and (b) an effective amount of a KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K1T17R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor). In some embodiments, the cancer comprises one or more cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein, respectively). Additionally or alternatively, in some embodiments, the cancer comprises one or more cells that have at least one mTOR-activating aberration. In some embodiments, the cancer is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin. In some embodiments, the cancer or tumor (such as any of the preceding cancers or tumors) is advanced, unresectable, and/or metastatic. In some embodiments, the cancer is solid tumor (e.g., advanced, unresectable, and/or metastatic solid tumor), lung cancer (e.g., advanced, unresectable, and/or metastatic lung cancer), or bladder cancer (e.g., advanced, unresectable, and/or metastatic bladder cancer). In some embodiments, the lung cancer is non-small cell lung cancer (NSCLC), e.g., advanced, unresectable, and/or metastatic NSCLC.

In some embodiments, provided is a method of treating a cancer in an individual (e.g., a human), comprising administering to the individual (a) an effective amount of a composition comprising nanoparticles comprising an mTOR inhibitor (such as a limus drug, e.g., sirolimus or a derivative or analog thereof) and an albumin (an “mTOR inhibitor nanoparticle composition”), wherein the mTOR inhibitor in the nanoparticles is associated (e.g., coated) with the albumin; and (b) an effective amount of a KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor). In some embodiments, the method comprises administering to the individual (a) an effective amount of a composition comprising nanoparticles comprising an mTOR inhibitor (such as a limus drug, e.g., sirolimus or a derivative or analog thereof) and an albumin (an “mTOR inhibitor nanoparticle composition”), wherein the nanoparticles have an average particle size of no greater than about 150 nm (such as no greater than about 120 nm); and (b) an effective amount of a KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor). In some embodiments, the method comprises administering to the individual (a) an effective amount of a composition comprising nanoparticles comprising an mTOR inhibitor (such as a limus drug, e.g., sirolimus or a derivative or analog thereof) and an albumin (an “mTOR inhibitor nanoparticle composition”), wherein the nanoparticles comprise the mTOR inhibitor associated (e.g., coated) with albumin, and wherein the nanoparticles have an average particle size of no greater than about 150 nm (such as no greater than about 120 nm); and (b) an effective amount of a KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor). In some embodiments, the method comprises administering to the individual (a) an effective amount of a composition comprising nanoparticles comprising an mTOR inhibitor (such as a limus drug, e.g., sirolimus or a derivative of analog thereof) and an albumin (an “mTOR inhibitor nanoparticle composition”), wherein the nanoparticles comprise the mTOR inhibitor associated (e.g., coated) with the albumin, wherein the nanoparticles have an average particle size of no greater than about 150 nm (such as no greater than about 120 nm, for example about 100 nm), and wherein the weight ratio of albumin and the mTOR inhibitor in the mTOR inhibitor nanoparticle composition is about 10:1 or less (such as about 10:1 or about 9:1 or about 8:1); and (b) an effective amount of a KRAS G12C inhibitor. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. Exemplary mTOR inhibitor nanoparticle compositions that find use with the methods provided herein are described in further detail below. In some embodiments, the cancer comprises one or more cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein). Additionally or alternatively, in some embodiments, the cancer comprises one or more cells that have at least one mTOR-activating aberration. Exemplary cancers that are treated according to a method described herein are described elsewhere herein.

In some embodiments, the KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor) is, e.g., a polypeptide (such as an antibody), a peptide, an antisense oligonucleotide or a small molecule that inhibits the activity of the KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein, respectively).

Additional information about exemplary agents that can be used in the treatment of cancers that comprises one or more cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein) can be found at, e.g., world-wide-web(dot)mycancergenome(dot)org/content/biomarkers/.

In some embodiments, the KRAS inhibitor is a KRAS G12C inhibitor. In some embodiments, the KRAS G12C inhibitor is a small molecule. Exemplary small molecule KRAS G12C inhibitors that find use with the methods provided herein include, without limitation, e.g., sotorasib, which is also known as AMG 510 (Amgen/Beigene), MRTX849, which is also known as adagrasib (Mirati/Zai Lab), JAB-21822 (Jacobiopharma), GDC-6036 (Genentech), JDQ443 (Novartis), D-1553 (InventisBio and Merck Sharp & Dohme), GH35 (Genhouse Bio), GFH925 (GenFleet Therapeutics), BPI-421286 (Bettapharma), LY3537982, RMC-6291 (Revolution Medicine), RMC-8839 (Revolution Medicine), HBI-2438 (Huya Biosciences International, LLC), and JNJ-74699157 (Johnson & Johnson). Exemplary small molecule KRAS G12D inhibitors that find use with the methods provided herein include, without limitation, MRTX1133 (Mirati Therapeutics) and RMC-6236 (Revolution Medicines). Exemplary small molecule KRAS G12V inhibitors that find use with the methods provided herein include, without limitation, JAB-23000. Additional details regarding these and other exemplary KRAS inhibitors are described in further detail below. In some embodiments, the mTOR inhibitor nanoparticle composition and the KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor) are administered sequentially (i.e., the administration periods of the mTOR inhibitor nanoparticle composition and the KRAS inhibitor do not overlap with each other). In some embodiments, mTOR inhibitor nanoparticle composition and the KRAS inhibitor are administered simultaneously. In some embodiments, mTOR inhibitor nanoparticle composition and the KRAS inhibitor are administered concurrently (i.e., the administration periods of the mTOR inhibitor nanoparticle composition and the KRAS inhibitor overlap with each other).

In some embodiments, provided is a method of treating a cancer in an individual (e.g., a human), comprising administering to the individual (a) an effective amount of a composition comprising nanoparticles comprising an mTOR inhibitor (such as a limus drug, e.g., sirolimus or a derivative or analog thereof) and an albumin (an “mTOR inhibitor nanoparticle composition”); and (b) an effective amount of a KRAS inhibitor (e.g., a KRAS GT2C inhibitor, a KRAS GT2A inhibitor, a KRAS GT2D inhibitor, a KRAS GT2F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K1T17N inhibitor, a KRAS K1T17R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor), wherein the mTOR inhibitor nanoparticle composition and the KRAS inhibitor are administered concurrently. In some embodiments, the cancer comprises one or more cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS GT3V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K1T17N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein, respectively). Additionally or alternatively, in some embodiments, the cancer comprises one or more cells that have at least one mTOR-activating aberration. In some embodiments, the administrations of the mTOR inhibitor nanoparticle composition and the KRAS inhibitor are initiated at about the same time (for example, within any one of 1, 2, 3, 4, 5, 6, or 7 days). In some embodiments, the administrations of the mTOR inhibitor nanoparticle composition and the KRAS inhibitor are terminated at about the same time (for example, within any one of 1, 2, 3, 4, 5, 6, or 7 days). In some embodiments, the administration of the KRAS inhibitor continues (for example for about any one of 0.5, 1, 2, 3, 4, 5, 6, 7, 8, 9, 10, 11, or 12 months) after the termination of the administration of the mTOR inhibitor nanoparticle composition. In some embodiments, the administration of the KRAS inhibitor is initiated after (for example after about any one of 0.5, 1, 2, 3, 4, 5, 6, 7, 8, 9, 10, 11, or 12 months) the initiation of the administration of the mTOR inhibitor nanoparticle composition. In some embodiments, the administrations of the mTOR inhibitor nanoparticle composition and the KRAS inhibitor are initiated and terminated at about the same time. In some embodiments, the administrations of the mTOR inhibitor nanoparticle composition and the KRAS inhibitor are initiated at about the same time and the administration of the KRAS inhibitor continues (for example for about any one of 0.5, 1, 2, 3, 4, 5, 6, 7, 8, 9, 10, 11, or 12 months) after the termination of the administration of the mTOR nanoparticle composition. In some embodiments, the administration of the mTOR nanoparticle composition and the KRAS inhibitor stop at about the same time and the administration of the KRAS inhibitor is initiated after (for example after about any one of 0.5, 1, 2, 3, 4, 5, 6, 7, 8, 9, 10, 11, or 12 months) the initiation of the administration of the mTOR inhibitor nanoparticle composition. In some embodiments, the administration of the nanoparticle composition and the KRAS inhibitor stop at about the same time and the administration of the mTOR inhibitor nanoparticle composition is initiated after (for example after about any one of 0.5, 1, 2, 3, 4, 5, 6, 7, 8, 9, 10, 11, or 12 months) the initiation of the administration of the KRAS inhibitor.

In some embodiments of any of the methods described herein, the individual (e.g., human) has been diagnosed with or is suspected of having cancer. In some embodiments, the cancer comprises one or more cells that express a KRAS mutant protein. Additionally or alternatively, in some embodiments, the cancer comprises one or more cells that have at least one mTOR-activating aberration. In some embodiments, the individual is a human. In some embodiments, the individual is a clinical patient, a clinical trial volunteer, an experimental animal, etc.

In some embodiments of any of the methods described herein, the method comprises (such as further comprises) selecting the individual for treatment based on the presence of one or more cancer cells with at least one mTOR-activating aberration in a sample from the individual prior to the administration of the mTOR inhibitor nanoparticle composition and the KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor). In some embodiments, the mTOR-activating aberration comprises a mutation of an mTOR-associated gene. In some embodiments, the mTOR-activating aberration comprises a copy number variation of an mTOR-associated gene. In some embodiments, the mTOR-activating aberration comprises an aberrant expression level of an mTOR-associated gene. In some embodiments, the mTOR-activating aberration comprises an aberrant activity level of an mTOR-associated gene. In some embodiments, the at least one mTOR-activating aberration comprises an aberrant phosphorylation level of the protein encoded by the mTOR-associated gene. In some embodiments, the mTOR-activating aberration is in at least one mTOR-associated gene selected from the group consisting of AKT1, FLT-3, MTOR, PIK3CA, PIK3CG, TSC1, TSC2, RHEB, STK11, NF1, NF2, TP53, FGFR4, BAP1, KRAS, NRAS, NRF2, KEAP1, and PTEN. In some embodiments, the at least one mTOR-associated gene is TSC1 and/or TSC2. In some embodiments, the mTOR-activating aberration is assessed (such as detected) by gene sequencing (e.g., next-generation sequencing or “NGS”). In some embodiments, mTOR-activating aberration is assessed (such as detected) by sequencing the DNA in a tumor sample (e.g., a formalin-fixed paraffin embedded tumor sample) from the individual. In some embodiments, the mTOR-activating aberration is assessed (such as detected) by sequencing circulating or cell-free DNA in a blood sample from the individual. Further details regarding mTOR-activating aberrations and assessing/detecting mTOR-activating aberrations in a sample (e.g., a tumor sample or blood sample) obtained from an individual having cancer are described herein below and in WO 2017/004267, the contents of which are incorporated by reference herein in their entirety.

In some embodiments of any of the methods described herein, the method comprises (such as further comprises) selecting the individual for treatment based on the presence of one or more cancer cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein) in a sample from the individual prior to the administration of the mTOR inhibitor nanoparticle composition and the KRAS inhibitor. In some embodiments, the KRAS mutation is assessed (such as detected) by gene sequencing (e.g., next-generation sequencing or “NGS”). In some embodiments, the KRAS mutation is assessed (such as detected) by sequencing the DNA in a cancer sample (e.g., a formalin-fixed paraffin embedded cancer sample) from the individual. In some embodiments, the KRAS mutation is assessed (such as detected) by sequencing circulating or cell-free DNA in a blood sample from the individual.

Cancer treatments can be evaluated, for example, by tumor regression, tumor weight or size shrinkage, time to progression (TTP), duration of survival (DOS), progression free survival (PFS), overall survival (OS), objective response rate (ORR), duration of response (DOR), quality of life (QoL), protein expression and/or activity. Approaches to determining efficacy of the therapy can be employed, including for example, measurement of response through radiological imaging. In some embodiments, the efficacy of treatment is measured as the percentage tumor growth inhibition (% TGI), calculated using the equation 100×(ΔC−ΔT)/ΔC, where ΔT and ΔC are the changes in the mean tumor volumes between the last day when all animals in the saline or control group were alive and the first day of measurement for the treatment and control groups, respectively. In some embodiments, the % TGI is about any one of 10%, 20%, 30%, 40%, 50%, 60%, 70%, 80%, 90%, 91%, 92%, 93%, 94%, 95%, or more than 95% (e.g., more than about any one of 96%, 97%, 98%, or 99%). In some embodiments, overall survival (OS) is defined as the time from the start of treatment to date of death due to any cause. In some embodiments, progression free survival (PFS) is defined as the time from the start of treatment to the date of progressive disease (PD) or death due to any cause, whichever occurs first. In some embodiments, duration of response (DOR) is defined as the time from the start of treatment to the first documentation of objective tumor response (complete response (“CR”) or partial response (“PR”)) to the first documentation of either PD or death due to any cause, whichever occurs first. In some embodiments, objective response rate (ORR) is defined as the proportion of individuals (e.g., patients, subjects) that experience confirmed complete response (CR) or partial response (PR) based on RECIST v1.1 criteria during the time period from first dose of treatment until last dose of treatment.

Compositions Comprising Nanoparticles Comprising an mTOR Inhibitor and an Albumin mTOR Inhibitors

The methods provided herein comprise administering an effective amount of an mTOR inhibitor nanoparticle composition to an individual having cancer. In some embodiments, the cancer comprises one or more cancer cells that express a KRAS mutant protein (e.g., (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein). Additionally or alternatively, in some embodiments, the cancer comprises one or more cells that have at least one mTOR-activating aberration. In some embodiments, “mTOR inhibitor” refers to inhibitors of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110: 163-75; Fang et al. (2001). Science 294 (5548): 1942-5). mTORCT integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GOL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto et al. (2004). Nat. Cell Biol. 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor (such as a limus drug, e.g., sirolimus or a derivative or analog thereof) is an inhibitor of mTORC1. In some embodiments, the mTOR inhibitor (such as a limus drug, e.g., sirolimus or a derivative or analog thereof) is an inhibitor of mTORC2. In some embodiments, the mTOR inhibitor (such as a limus drug, e.g., sirolimus or a derivative or analog thereof) is an inhibitor of both mTORC1 and mTORC2.

In some embodiments, the mTOR inhibitor is a limus drug, which includes sirolimus (also known as rapamycin and Rapamune) and its derivatives and analogs. Other exemplary limus drugs include, but are not limited to, temsirolimus (also known as CCI-779 and Torisel), everolimus (also known as RAD001, Zortress, Certican, and Afinitor), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is sirolimus (rapamycin). Sirolimus is macrolide antibiotic that complexes with FKBP-12 and inhibits the mTOR pathway by binding mTORC1.

Other exemplary mTOR inhibitors include, but are not limited to, BEZ235 (NVP-BEZ235), AZD8055, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, and WYE-354.

BEZ235 (NVP-BEZ235) is an imidazoquilonine derivative that is an mTORC1 catalytic inhibitor (Roper J, et al. PLoS One, 2011, 6(9), e25132). Everolimus is the 40-O-(2-hydroxyethyl) derivative of sirolimus and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORC1 complex. PI-103 is a small molecule that inhibits the activation of the rapamycin-sensitive (mTORC1) complex (Knight et al. (2006) Cell. 125: 733-47). KU-0063794 is a small molecule that inhibits the phosphorylation of mTORC1 at Ser2448 in a dose-dependent and time-dependent manner. INK 128, AZD2014, NVP-BGT226, CH5132799, WYE-687, and are each small molecule inhibitors of mTORC1. PF-04691502 inhibits mTORC1 activity. GDC-0980 is an orally bioavailable small molecule that inhibits Class I P13 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Kγ and mTOR. PP-121 is a multi-target inhibitor of PDGFR, Hck, mTOR, VEGFR2, Src and Abl. OSI-027 is a selective and potent dual inhibitor of mTORC1 and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3Kβ, PI3Kδ, PI3Kγ and mTOR. WYE-354 inhibits mTORC1 in HEK293 cells (0.2 μM-5 μM) and in HUVEC cells (10 nM-1 μM). WYE-354 is a potent, specific and ATP-competitive inhibitor of mTOR. Deforolimus (Ridaforolimus, AP23573, MK-8669) is a selective mTOR inhibitor.

In some embodiments, the mTOR inhibitor is a derivative or analog of any of the mTOR inhibitors described herein. In some embodiments, a “derivative” or “analog” of an mTOR inhibitor refers to a compound that is structurally similar to the mTOR inhibitor or is in the same general chemical class as the mTOR inhibitor. In some embodiments, the derivative or analog of the mTOR inhibitor retains similar chemical and/or physical property (including, for example, functionality) of the second therapeutic agent or moiety.

In some embodiments, provided is a method of treating a cancer in an individual (e.g., a human), comprising administering to the individual (a) an effective amount of a composition comprising nanoparticles comprising an mTOR inhibitor and an albumin (an “mTOR inhibitor nanoparticle composition”), wherein the mTOR inhibitor is selected from the group consisting of sirolimus (also known as rapamycin and Rapamune) or a derivative or analog thereof, temsirolimus (also known as CCI-779 and Torisel), everolimus (also known as RAD001, Zortress, Certican, and Afinitor), ridaforolimus (AP-23573), deforolimus (MK-8669), zotarolimus (ABT-578), pimecrolimus, and tacrolimus (FK-506), BEZ235 (NVP-BEZ235), AZD8055, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, and WYE-354; and (b) an effective amount of a KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor). In some embodiments, the mTOR inhibitor is sirolimus. In some embodiments, the cancer comprises one or more cancer cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS GT3V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein, respectively). Additionally or alternatively, in some embodiments, the cancer comprises one or more cells that have at least one mTOR-activating aberration. In some embodiments, cancer is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin. In some embodiments, the cancer or tumor (such as any of the preceding cancers or tumors) is advanced, unresectable, and/or metastatic. In some embodiments, the cancer is solid tumor (e.g., advanced, unresectable, and/or metastatic solid tumor), lung cancer (e.g., advanced, unresectable, and/or metastatic lung cancer), or bladder cancer (e.g., advanced, unresectable, and/or metastatic bladder cancer). In some embodiments, the lung cancer is non-small cell lung cancer (NSCLC), e.g., advanced, unresectable, and/or metastatic NSCLC. An mTOR inhibitor nanoparticle composition (such as sirolimus/albumin nanoparticle composition) can be administered via any of the accepted modes of administration or agents known in the art. In some embodiments, the mTOR inhibitor nanoparticle composition is administered intravenously or subcutaneously. In some embodiments, the mTOR inhibitor nanoparticle composition (such as sirolimus/albumin nanoparticle composition) and the KRAS inhibitor are administered in a single unit dose. In some embodiments, the mTOR inhibitor nanoparticle composition (such as sirolimus/albumin nanoparticle composition) and the KRAS inhibitor are administered in separate dosage forms.

In some embodiments, provided is a method of treating a cancer in an individual (e.g., a human), comprising administering to the individual (a) an effective amount of a composition comprising nanoparticles comprising an mTOR inhibitor (e.g., sirolimus or a derivative or analog thereof) and an albumin, and (b) a KRAS G12C inhibitor (e.g., sotorasib or adagrsib). In some embodiments, the nanoparticles are administered (e.g., intravenously or subcutaneously administered) once a week, twice every three weeks (e.g., Day 1 and Day 8 of a 21-day cycle), or once every three weeks. In some embodiments, the amount of the mTOR inhibitor (e.g., sirolimus) for each administration is about 1-75 mg/m². In some embodiments, the KRAS G12C inhibitor is administered (e.g., orally) once a day or twice a day. In some embodiments, the amount of the KRAS G12C inhibitor for each administration is about 200-800 mg, optionally wherein the KRAS G12C inhibitor is administered twice a day (e.g., between a 12-hour interval). In some embodiments, the amount of the KRAS G12C inhibitor for each administration is about 100-2000 mg, optionally wherein the KRAS G12C inhibitor is administered once a day. In some embodiments, the cancer is a locally advanced or metastatic cancer. In some embodiments, the cancer is a solid tumor (e.g., a lung cancer, e.g., NSCLC, e.g., bladder cancer). In some embodiments, the cancer comprises a mTOR-activating aberration in STK11, TP53, ATM, CDKN2A, or UGT2B17. In some embodiments, the cancer comprises a mTOR-activating aberration in one or more genes selected from the group consisting of TP53, STK11, PTEN, ATM, CDKN2A, and UGT2B17.

### Nanoparticle Compositions

The mTOR inhibitor nanoparticle compositions that find use with the methods described herein comprise nanoparticles comprising (in various embodiments consisting essentially of or consisting of) an mTOR inhibitor (such as a limus drug, e.g., sirolimus or a derivative or analog thereof) and an albumin (such as human serum albumin). Nanoparticles of poorly water soluble drugs (such as macrolides) have been disclosed, for example, in U.S. Pat. Nos. 5,916,596; 6,506,405; 6,749,868, 6,537,579, 7,820,788, and 8,911,786, and also in U.S. Pat. Pub. Nos. 2006/0263434, and 2007/0082838; PCT Patent Application WO08/137148, each of which is incorporated herein by reference in their entirety.

In some embodiments, the mTOR inhibitor nanoparticle composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 10 to about 400 nm. In some embodiments, the average or mean diameter of the nanoparticles is about 10 to about 150 nm. In some embodiments, the average or mean diameter of the nanoparticles is about 40 to about 120 nm. In some embodiments, the nanoparticles are no less than about 50 nm. In some embodiments, the nanoparticles are sterile-filterable.

In some embodiments, the nanoparticles in the mTOR inhibitor nanoparticle composition have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the mTOR inhibitor nanoparticle composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the mTOR inhibitor nanoparticle composition fall within the range of about 10 nm to about 400 nm, including for example about 10 nm to about 200 nm, about 20 nm to about 200 nm, about 30 nm to about 180 nm, about 40 nm to about 150 nm, about 40 nm to about 120 nm, and about 60 nm to about 100 nm.

In some embodiments, the albumin in the mTOR inhibitor nanoparticle composition has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are cross-linked (for example cross-linked through one or more disulfide bonds).

In some embodiments, the nanoparticles comprising an mTOR inhibitor described herein (such as a limus drug, e.g., sirolimus or a derivative or analog thereof) are associated (e.g., coated) with an albumin (such as human albumin or human serum albumin). In some embodiments, the composition comprises an mTOR inhibitor (such as a limus drug, e.g., sirolimus or a derivative or analog thereof) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as a limus drug, e.g., sirolimus or a derivative or analog thereof) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as a limus drug, e.g., sirolimus or a derivative or analog thereof) that is substantially free of polymeric materials (such as polymeric matrix).

In some embodiments, the mTOR inhibitor nanoparticle composition comprises an albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of an albumin (such as human albumin or human serum albumin) and an mTOR inhibitor (such as a limus drug, e.g., sirolimus or a derivative or analog thereof) in an mTOR inhibitor nanoparticle composition is about 18:1 or less, such as about 15:1 or less, for example about 10:1 or less. In some embodiments, the weight ratio of an albumin (such as human albumin or human serum albumin) and an mTOR inhibitor (such as a limus drug, e.g., sirolimus or a derivative or analog thereof) in the in an mTOR inhibitor nanoparticle composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of an albumin and an mTOR inhibitor (such as a limus drug, e.g., sirolimus or a derivative or analog thereof) in the nanoparticle portion of the in an mTOR inhibitor nanoparticle composition is about any one of 1:2, 1:3, 1:4, 1:5, 1:9, 1:10, 1:15, or less. In some embodiments, the weight ratio of the albumin (such as human albumin or human serum albumin) and the mTOR inhibitor (such as a limus drug, e.g., sirolimus or a derivative or analog thereof) in the in an mTOR inhibitor nanoparticle composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the mTOR inhibitor nanoparticle composition (such as sirolimus/albumin nanoparticle composition) comprises one or more of the above characteristics.

In some embodiments, the mTOR inhibitor nanoparticle composition that finds use with the methods described herein is in a dry formulation (such as lyophilized composition). In some embodiments, the mTOR inhibitor nanoparticle composition is suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the mTOR inhibitor nanoparticle composition comprises an albumin (such as human albumin or human serum albumin). The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mr 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolemic shock (see, e.g., Tullis, JAMA, 237: 355-360, 460-463, (1977)) and Houser et al., Surgery, Gynecology and Obstetrics, 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The albumin (such as human albumin or human serum albumin) in the mTOR inhibitor nanoparticle composition generally serves as a carrier for the mTOR inhibitor, i.e., the albumin in the composition makes the mTOR inhibitor (such as a limus drug, e.g., sirolimus or a derivative or analog thereof) more readily suspendable in an aqueous medium or helps maintain the suspension as compared to compositions not comprising an albumin. This can avoid the use of toxic solvents (or surfactants) for solubilizing the mTOR inhibitor, and thereby can reduce one or more side effects of administration of the mTOR inhibitor (such as a limus drug, e.g., sirolimus or a derivative thereof) into an individual (such as a human). Thus, in some embodiments, an mTOR inhibitor nanoparticle composition that finds use with the methods described herein is substantially free (such as free) of surfactants, such as Cremophor (or polyoxyethylated castor oil, including Cremophor EL® (BASF)). In some embodiments, the mTOR inhibitor nanoparticle composition (such as sirolimus/albumin nanoparticle composition) is substantially free (such as free) of surfactants. A composition is “substantially free of Cremophor” or “substantially free of surfactants” if the amount of Cremophor or surfactant in the composition is not sufficient to cause one or more side effect(s) in an individual when the mTOR inhibitor nanoparticle composition (such as sirolimus/albumin nanoparticle composition) is administered to the individual. In some embodiments, the mTOR inhibitor nanoparticle composition (such as sirolimus/albumin nanoparticle composition) contains less than about any one of 20%, 15%, 10%, 7.5%, 5%, 2.5%, or 1% organic solvent or surfactant. In some embodiments, the albumin is human albumin or human serum albumin. In some embodiments, the albumin is recombinant albumin.

The amount of an albumin in an mTOR inhibitor nanoparticle composition that finds use with the methods described herein will vary depending on other components in the composition. In some embodiments, the mTOR inhibitor nanoparticle composition comprises an albumin in an amount that is sufficient to stabilize the mTOR inhibitor (such as a limus drug, e.g., sirolimus or a derivative or analog thereof) in an aqueous suspension, for example, in the form of a stable colloidal suspension (such as a stable suspension of nanoparticles). In some embodiments, the albumin is in an amount that reduces the sedimentation rate of the mTOR inhibitor (such as a limus drug, e.g., sirolimus or a derivative thereof) in an aqueous medium. For particle-containing compositions, the amount of the albumin also depends on the size and density of nanoparticles of the mTOR inhibitor.

An mTOR inhibitor (such as a limus drug, e.g., sirolimus or a derivative or analog thereof) is “stabilized” in an aqueous suspension if it remains suspended in an aqueous medium (such as without visible precipitation or sedimentation) for an extended period of time, such as for at least about any one of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000× magnification about fifteen minutes after preparation of the suspension. Stability can also be evaluated under accelerated testing conditions, such as at a temperature that is higher than about 40° C.

In some embodiments, the albumin is present in the mTOR inhibitor nanoparticle composition in an amount that is sufficient to stabilize the mTOR inhibitor (such as a limus drug, e.g., sirolimus or a derivative or analog thereof) in an aqueous suspension at a certain concentration. For example, the concentration of the mTOR inhibitor (such as a limus drug, e.g., sirolimus or a derivative or analog thereof) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as a limus drug, e.g., sirolimus or a derivative or analog thereof) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the mTOR inhibitor nanoparticle composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the mTOR inhibitor nanoparticle composition, in liquid form, comprises about 0.5% to about 5% (w/v) of albumin.

In some embodiments, the weight ratio of the albumin to the mTOR inhibitor (such as a limus drug, e.g., sirolimus or a derivative or analog thereof) in the mTOR inhibitor nanoparticle composition is such that a sufficient amount of mTOR inhibitor binds to, or is transported by, the cell. While the weight ratio of an albumin to an mTOR inhibitor (such as a limus drug, e.g., sirolimus or a derivative or analog thereof) will have to be optimized for different albumin and mTOR inhibitor combinations, generally the weight ratio of an albumin to an mTOR inhibitor (such as a limus drug, e.g., sirolimus or a derivative or analog thereof) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 11:1, about 5:1 to about 10:1, or about 10:1. In some embodiments, the albumin to mTOR inhibitor (such as a limus drug, e.g., sirolimus or a derivative or analog thereof) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as a limus drug, e.g., sirolimus or a derivative or analog thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the mTOR inhibitor nanoparticle composition to be administered to an individual (such as a human) without significant side effects. In some embodiments, the albumin (such as human serum albumin or human albumin) is in an amount that is effective to reduce one or more side effects of administration of the mTOR inhibitor (such as a limus drug, e.g., sirolimus or a derivative or analog thereof) to a human. In some embodiments, the term “reducing one or more side effects” of administration of the mTOR inhibitor (such as a limus drug, e.g., sirolimus or a derivative or analog thereof) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as a limus drug, e.g., sirolimus or a derivative or analog thereof) can be reduced.

In some embodiments, the mTOR inhibitor nanoparticle compositions that find use with the methods described herein comprise nanoparticles comprising an mTOR inhibitor (such as a limus drug, e.g., sirolimus or a derivative or analog thereof) and an albumin (such as human albumin or human serum albumin), wherein the nanoparticles have an average diameter of no greater than about 200 nm. In some embodiments, the mTOR inhibitor nanoparticle compositions that find use with the methods described herein comprise nanoparticles comprising an mTOR inhibitor (such as a limus drug, e.g., sirolimus or a derivative or analog thereof) and an albumin (such as human albumin or human serum albumin), wherein the nanoparticles have an average diameter of no greater than about 150 nm. In some embodiments, the mTOR inhibitor nanoparticle compositions that find use with the methods described herein comprise nanoparticles comprising an mTOR inhibitor (such as a limus drug, e.g., sirolimus or a derivative or analog thereof) and an albumin (such as human albumin or human serum albumin), wherein the nanoparticles have an average diameter of no greater than about 150 nm (for example about 100 nm). In some embodiments, the mTOR inhibitor nanoparticle compositions that find use with the methods described herein comprise nanoparticles comprising sirolimus and human albumin (such as human serum albumin), wherein the nanoparticles have an average diameter of no greater than about 150 nm (for example about 100 nm). In some embodiments, the mTOR inhibitor nanoparticle compositions that find use with the methods described herein comprise nanoparticles comprising sirolimus and human albumin (such as human serum albumin), wherein the average or mean diameter of the nanoparticles is about 10 to about 150 nm. In some embodiments, the mTOR inhibitor nanoparticle compositions that find use with the methods described herein comprise nanoparticles comprising sirolimus and human albumin (such as human serum albumin), wherein the average or mean diameter of the nanoparticles is about 40 to about 120 nm.

In some embodiments, the mTOR inhibitor nanoparticle compositions that find use with the methods described herein comprise nanoparticles comprising an mTOR inhibitor (such as a limus drug, e.g., sirolimus or a derivative or analog thereof) and an albumin (such as human albumin or human serum albumin), wherein the nanoparticles have an average diameter of no greater than about 200 nm, wherein the weight ratio of the albumin and the mTOR inhibitor (e.g., sirolimus) in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) and an albumin (such as human albumin or human serum albumin), wherein the nanoparticles have an average diameter of no greater than about 150 nm, wherein the weight ratio of the albumin and the mTOR inhibitor (e.g., sirolimus) in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) and an albumin (such as human albumin or human serum albumin), wherein the nanoparticles have an average diameter of about 150 nm, wherein the weight ratio of the albumin and the mTOR inhibitor (e.g., sirolimus) in the composition is no greater than about 10:1 (such as about 10:1 or about 9:1 or about 8:1). In some embodiments, the mTOR inhibitor nanoparticle compositions that find use with the methods described herein comprise nanoparticles comprising sirolimus and human albumin (such as human serum albumin), wherein the nanoparticles have an average diameter of no greater than about 150 nm (for example about 100 nm), wherein the weight ratio of albumin and mTOR inhibitor in the composition is about 10:1 or about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin). In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the nanoparticles have an average diameter of no greater than about 200 nm. In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the nanoparticles have an average diameter of no greater than about 150 nm. In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the nanoparticles have an average diameter of about 10 nm to about 150 nm. In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the nanoparticles have an average diameter of about 40 nm to about 120 nm. In some embodiments, the mTOR inhibitor nanoparticle compositions that find use with the methods described herein comprise nanoparticles comprising sirolimus associated (e.g., coated) with human albumin (such as human serum albumin), wherein the nanoparticles have an average diameter of no greater than about 150 nm (for example about 100 nm). In some embodiments, the mTOR inhibitor nanoparticle compositions that find use with the methods described herein comprise nanoparticles comprising sirolimus associated (e.g., coated) with human albumin (such as human serum albumin), wherein the nanoparticles have an average diameter of about 10 nm to about 150 nm. In some embodiments, the mTOR inhibitor nanoparticle compositions that find use with the methods described herein comprise nanoparticles comprising sirolimus associated (e.g., coated) with human albumin (such as human serum albumin), wherein the nanoparticles have an average diameter of about 40 nm to about 120 nm.

In some embodiment, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the nanoparticles have an average diameter of no greater than about 200 nm, wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the nanoparticles have an average diameter of no greater than about 150 nm, wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) associated (e.g., coated) with an albumin (such as human albumin or human serum albumin), wherein the nanoparticles have an average diameter of about 150 nm, wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising sirolimus associated (e.g., coated) with human albumin (such as human serum albumin), wherein the nanoparticles have an average diameter of no greater than about 150 nm (for example about 100 nm), wherein the weight ratio of albumin and the sirolimus in the composition is about 10:1 or about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin). In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin), wherein the nanoparticles have an average diameter of no greater than about 200 nm. In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin), wherein the nanoparticles have an average diameter of no greater than about 150 nm. In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin), wherein the nanoparticles have an average diameter of no greater than about 150 nm (for example about 100 nm). In some embodiments, the mTOR inhibitor nanoparticle compositions that find use with the methods described herein comprise nanoparticles comprising sirolimus stabilized by human albumin (such as human serum albumin), wherein the nanoparticles have an average diameter of no greater than about 150 nm (for example about 100 nm). In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin), wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin), wherein the nanoparticles have an average diameter of no greater than about 200 nm, wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin), wherein the nanoparticles have an average diameter of no greater than about 150 nm, wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising an mTOR inhibitor (such as a limus drug, e.g., sirolimus or a derivative or analog thereof) stabilized by an albumin (such as human albumin or human serum albumin), wherein the nanoparticles have an average diameter of about 150 nm, wherein the weight ratio of the albumin and the mTOR inhibitor in the composition is no greater than about 10:1 (such as about 10:1 or about 9:1 or about 8:1). In some embodiments, the mTOR inhibitor nanoparticle compositions that find use with the methods described herein comprise nanoparticles comprising sirolimus stabilized by human albumin (such as human serum albumin), wherein the nanoparticles have an average diameter of no greater than about 150 nm (for example about 100 nm), wherein the weight ratio of albumin and the sirolimus in the composition is about 10:1 or about 9:1 or about 8:1. In some embodiments, the average or mean diameter of the nanoparticles is about 10 nm to about 150 nm. In some embodiments, the average or mean diameter of the nanoparticles is about 40 nm to about 120 nm.

In some embodiments, the mTOR inhibitor nanoparticle compositions that find use with the methods described herein comprise nab-sirolimus. In some embodiments, the mTOR inhibitor nanoparticle composition that finds use with the methods described herein is nab-sirolimus. nab-sirolimus is a formulation of sirolimus stabilized by human albumin USP, which can be dispersed in directly injectable physiological solution. The weight ratio of human albumin and sirolimus is about 8:1 to about 10:1. When dispersed in a suitable aqueous medium such as 0.9% sodium chloride injection or 5% dextrose injection, nab-sirolimus forms a stable colloidal suspension of sirolimus. The mean particle size of the nanoparticles in the colloidal suspension is about 100 nanometers. Since HSA is freely soluble in water, nab-sirolimus can be reconstituted in a wide range of concentrations ranging from dilute (0.1 mg/ml sirolimus or a derivative thereof) to concentrated (20 mg/ml sirolimus or a derivative thereof), including for example about 2 mg/ml to about 8 mg/ml, or about 5 mg/ml.

Methods of making nanoparticle compositions are known in the art. For example, nanoparticles containing an mTOR inhibitor (such as a limus drug, e.g., sirolimus or a derivative or analog thereof) and an albumin (such as human serum albumin or human albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579, 7,820,788, and 8,911,786, and also in U.S. Pat. Pub. Nos. 2007/0082838, 2006/0263434 and PCT Application WO 08/137148.

Briefly, the mTOR inhibitor (such as a limus drug, e.g., sirolimus or a derivative or analog thereof) is dissolved in an organic solvent, and the solution can be added to an albumin solution. The mixture is subjected to high pressure homogenization. The organic solvent can then be removed by evaporation. The dispersion obtained can be further lyophilized. Suitable organic solvents include, for example, ketones, esters, ethers, chlorinated solvents, and other solvents known in the art. For example, the organic solvent can be methylene chloride or chloroform/ethanol (for example with a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1).

Other Components in mTOR Inhibitor Nanoparticle Compositions

In some embodiments, an mTOR inhibitor nanoparticle composition that finds use with the methods described herein is present in a composition that include other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the mTOR inhibitor nanoparticle composition that finds use with the methods described herein is suitable for administration to a human. In some embodiments, the composition is suitable for administration to a mammal such as, in the veterinary context, domestic pets and agricultural animals. There are a wide variety of suitable formulations of the mTOR inhibitor nanoparticle composition (such as sirolimus/albumin nanoparticle composition) (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets, or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. The formulations can additionally include lubricating agents, wetting agents, emulsifying, and suspending agents, preserving agents, sweetening agents, or flavoring agents.

Formulations suitable for parenteral administration include aqueous and non-aqueous, isotonic sterile injection solutions, which can contain antioxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the mTOR inhibitor nanoparticle composition that finds use with the methods described herein is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Albumin-Based Nanoparticle Compositions of Sirolimus (Rapamycin)

In some embodiments, the mTOR inhibitor nanoparticle composition that finds use with the methods described herein includes (a) nanoparticles that include sirolimus and albumin, and (b) a non-nanoparticle portion that includes sirolimus and albumin. The sirolimus and the albumin of the nanoparticles are associated with each other in the nanoparticles. For example, the nanoparticles may include a coating having the albumin, which surrounds a core comprising the sirolimus. In the non-nanoparticle portion of the composition, the sirolimus and the albumin may or may not associated with each other (i.e., the sirolimus may be in a reversible binding equilibrium with the albumin), but do not associate with each other in a manner that forms nanoparticles. That is, the nanoparticle composition may include nanoparticle-bound albumin and nanoparticle-bound sirolimus in the nanoparticle portion of the composition, and non-nanoparticle albumin and non-nanoparticle sirolimus in the non-nanoparticle portion of the composition. As used herein, “in the nanoparticles” is used synonymously with “in the nanoparticle portion.” The albumin of the nanoparticles may be further distinguishable from the albumin in the non-nanoparticle portion of the composition; for example, the oligomeric profile of the albumin in the nanoparticles may differ from the oligomeric profile of the albumin in the non-nanoparticle portion of the composition. The oligomer profile means the percentage of various albumin species compared with the total albumin in the composition. The types of albumin species includes albumin monomers, dimers, trimers, oligomers, and polymers. As used herein, “albumin monomers” or “monomeric albumin” refers to an albumin species having one, and only one, albumin unit; “albumin dimers” or “dimeric albumin” refers to an albumin species having two, and only two, albumin units; “albumin trimers” or “trimeric albumin” refers to albumin species having three, and only three, albumin units; “albumin polymers” refers to albumin species having a higher molecular weight than albumin monomers and albumin dimers; “albumin oligomers” or “oligomeric albumin” refers to lower molecular weight polymeric albumin species associated with a UV-based size-exclusion chromatography peak observed between a peak associated with albumin dimers and higher molecular weight polymeric albumin species.

The albumin of the nanoparticles associates with the sirolimus of the nanoparticles so that a nanoparticle suspension has a high concentration of sirolimus, which allows the composition to be used as a pharmaceutical composition for treating certain diseases, such as cancer. Manufactured nanoparticles (which may be made, for example, using the methods described herein) may be formulated, filtered, or otherwise processed to obtain the pharmaceutical composition, which may be suitable for medical use in a human individual.

Generally, to make the sirolimus pharmaceutical compositions described herein, sirolimus is dissolved in an organic solvent. Suitable organic solvents include, for example, ketones, esters, ethers, chlorinated solvents, and other solvents known in the art. For example, the organic solvent can be a mixture of methylene chloride/ethanol, chloroform/ethanol, or chloroform/tert-butanol (for example with a ratio of about any one of 1:9, 1:8, 1:7, 1:6, 1:5, 1:4, 1:3, 1:2, 1:1, 2:1, 3:1, 4:1, 5:1, 6:1, 7:1, 8:1, or 9:1 or with a ratio of about any one of 3:7, 5:7, 4:6, 5:5, 6:5, 8:5, 9:5, 9.5:5, 5:3, 7:3, 6:4, or 9.5:0.5). In some embodiments, the organic solvent comprises between about 10% and about 50% tert-butanol by volume. In some embodiments, the organic solvent comprises about any of 10%, 15%, 20%, 25%, 30%, 35%, 40%, 45%, or 50% tert-butanol by volume. In some embodiments, the organic solvent comprises about any of 10-15%, 15-20%, 20-25%, 25-30%, 30-35%, 35-40%, 40-45%, or 45-50%, or any combination of such ranges, of tert-butanol by volume. In some embodiments, the organic solvent comprises between about 50% and about 90% chloroform by volume. In some embodiments, the organic solvent comprises about any of 50%, 55%, 60%, 65%, 70%, 75%, 80%, 85%, or 90% chloroform by volume. In some embodiments, the organic solvent comprises about any of 50-55%, 55-60%, 60-65%, 65-70%, 70-75%, 75 80%, 80-85%, or 85-90%, or any combination of such ranges, of chloroform by volume. In some embodiments, the organic solvent comprises between about 10% and about 50% tert-butanol by volume and between about 50% and about 90% chloroform by volume. In some embodiments, the organic solvent comprises chloroform and tert-butanol at a volumetric ratio of about 1:1 to about 1:9, such as about any of 1:1, 2:1, 3:1, 4:1, 5:1, 6:1, 7:1, 8:1, and 9:1.

Albumin (such as recombinant albumin, for example NOVOZYME™ recombinant albumin or INTRIVIA™ recombinant albumin disclosed herein) is dissolved in an aqueous solution (such as water) and combined with the sirolimus solution to form a crude emulsion. The mixture is subjected to high pressure homogenization (e.g., using an Avestin, APV Gaulin, MICROFLUIDIZER™ such as a MICROFLUIDIZER™ Processor M-110EH from Microfluidics, Stansted, or Ultra Turrax homogenizer). The emulsion may be cycled through the high pressure homogenizer for between about 2 to about 100 cycles, such as about 5 to about 50 cycles or about 6 to about 20 cycles (e.g., about any one of 6, 8, 10, 12, 14, 16, 18 or 20 cycles). The organic solvent can then be removed by evaporation utilizing suitable equipment known for this purpose, including, but not limited to, rotary evaporators, falling film evaporators, wiped film evaporators, spray driers, and the like that can be operated in batch mode or in continuous operation. In some embodiments, the evaporator is a wiped film evaporator. The solvent may be removed at reduced pressure (such as at about any one of 25 mm Hg, 30 mm Hg, 40 mm Hg, 50 mm Hg, 100 mm Hg, 200 mm Hg, or 300 mm Hg). The amount of time used to remove the solvent under reduced pressure may be adjusted based on the volume of the formulation. For example, for a formulation produced on a 300 mL scale, the solvent can be removed at about 1 to about 300 mm Hg (e.g., about any one of 5-100 mm Hg, 10-50 mm Hg, 20-40 mm Hg, or 25 mm Hg) for about 5 to about 60 minutes (e.g., about any one of 7, 8, 9, 10, 11, 12, 13, 14, 15 16, 18, 20, 25, or 30 minutes). The dispersion obtained can be further lyophilized.

The nanoparticle compositions comprising sirolimus and an albumin (such as pharmaceutical compositions) described herein can be in liquid (e.g., as a nanoparticle suspension) or powder forms. For example, in some embodiments, the composition is a liquid nanoparticle suspension (for example prior to lyophilization). In some embodiments, the composition is a reconstituted suspension (e.g., in an aqueous solution such as a saline solution). In some embodiments, the composition is dried, such as lyophilized. In some embodiments, the composition is sterile. In some embodiments, the composition is contained in a sealed container, such as a sealed vial (e.g., a glass vial) or sealed bag.

Additional details regarding sirolimus/albumin nanoparticle compositions are provided in PCT/US2020/060070, the contents of which are incorporated herein in their entirety.

An exemplary sirolimus/albumin nanoparticle composition is FYARRO™, also known as sirolimus protein-bound particles for injectable suspension (albumin-bound). Complete information about sirolimus protein-bound particles for injectable suspension (albumin-bound) (i.e., FYARRO™) preparation, dispensing, dosage, and administration schedule can be found in the local package insert (for the United States, see, e.g., www(dot)accessdata(dot)fda.gov/drugsatfda_docs/label/2021/2133121bl(dot)pdf.

### KRAS and Exemplary KRAS Inhibitors

KRAS, a member of the RAS family, is a key regulator of signaling pathways responsible for cell proliferation, differentiation, and survival. See, e.g., Cox et al. (2003) Nat Rev Drug Discov. 13(11):828-851 and Downward J. (2003) Nat Rev Cancer. 3(1):11-22. KRAS is the most frequently mutated oncogene in human cancer, and mutations in KRAS can result in continuous cellular proliferation and cancer development. The distribution of KRAS mutations varies in different human cancers. At the allele level, most mutations by single-nucleotide substitutions occurred at one of five “hotspot” codons: 12, 13, 61, 117, and 146. Glycine 12 can be mutated to at least eight amino acids (A, C, D, F, L, R, S, and V). Glycine 13 can be mutated to at least seven amino acids (A, C, F, P, R, S, and V) Glutamine 61 can be mutated to at least six other amino acids (E, H, K, L, P, and R) and a stop codon. Lysine 117 can be mutated to at least two other amino acids (N and R). Alanine 146 can be mutated to one of at least six amino acids (E, G, P, S, T, and V). In some embodiments, the term “KRAS inhibitor” refers to any agent, e.g., polypeptide, fusion polypeptide, antibody, peptide, antisense oligonucleotide, or small molecule drug, that inhibits the activity of the KRAS mutant protein.

### KRAS G12C Inhibitors

The KRAS G12C mutation occurs in about 13% of NSCLC patients, and 1%-3% of colorectal and other solid tumors. G12C is a single point mutation with a glycine-to-cysteine substitution at codon 12. See Cox et al. (2003) Nat Rev Drug Discov. 13(11):828-851; Neumann et al. (2009) Pathol Res Pract. 205:858-862; and Biernacka et al. (2016) Cancer Genet. 209(5):195-198. This substitution favors the activated GTP-bound state of KRAS, amplifying signaling pathways that lead to oncogenesis (see, e.g., Ryan et al. (2018) Nat Rev Clin Oncol. 15(11):709-720).

In some embodiments, the term “KRAS G12C inhibitor” refers to any agent, e.g., polypeptide, fusion polypeptide, antibody, peptide, antisense oligonucleotide, or small molecule drug, that inhibits the activity of the KRAS G12C mutant protein. In some embodiments, the KRAS G12C inhibitor interacts directly with the KRAS G12C mutant protein to inhibit the protein's activity. In some embodiments, the KRAS G12C inhibitor is a small molecule drug. Exemplary small molecule KRAS G12C inhibitors that find use with the methods provided herein include, without limitation, e.g., sotorasib (also known as AMG 510, LUMAKRAS™, and LUMYKRAS™), adagrasib (also known as MRTX849), JAB-21822 (also known as JAB-21000), GDC-6036, JDQ443, D-1553, GH35, GFH925, BPI-421286, LY3537982, RMC-6291, RMC-8839, HBI-2438, or JNJ-74699157. Other exemplary small molecule KRAS G12C inhibitors are described in, e.g., Hillig et al. (2019) Proc Natl Acad Sci USA. 116(7): 2551-2560 and Sun et al. (2012) Angew Chem Int Ed Engl. 51(25): 6140-6143, and in WO 2020/233592, WO 2021/023247, WO 2021/083167, WO 2020/238791, WO 2021/000885, CN 112585129, CN 112552295, CN 112390818, CN 112390796, WO 2019/141250, CN 111592528, WO 2020/259432, CN 112300153, CN 112552294, WO 2020/239123, CN 110698378, CN 111377918, CN 111205286, CN 112047939, WO 2020/259513, WO 2021/023154, CN 112430234, WO 2021/063346, WO 2021/058018, CN 112225734, WO 2021/155716, CN112159405, CN 112778302, CN 112830928, WO 2020/1027943, CN 112574199, WO 2021/037018, CN 110172089, WO 2020/156285, CN 111499634, WO 2020/177629, WO 2020/216190. WO 2020/221239, WO 2020/233592, WO 2020/238791, WO 2020/239077, WO 2020/239123, CN 112047933, CN 112047937, CN 112047948, WO 2020/259513, WO 2020/259573, WO 2020/259432, WO 2021/000885, CN 112174950, CN 112300153, WO 2021/023154, WO 2021/023247, CN 112390818, WO 2021/027943, WO 2021/027911, WO 2021/031952, CN 112390796, WO 2021/037018, CN 112442029, WO 2021/043322, WO 2021/052499, CN 112538084, CN 112552295, WO 2021/058018, WO 2021/063346, WO 2021/068898, WO 2021/078285, CN 112225734, CN 112707905, CN 112745335, WO 2021/083167, CN 112778284, WO 2021/088458, CN 112851663, WO 2021/093758, WO 2021/098859, CN 112830928, CN 111377918, WO 2021/104431, WO 2021/109737, WO 2021/113595, CN 112920183, WO 2021/121371, WO 2021/121367, CN 113004269, WO 2021/129824, WO 2021/129820, CN 113061132, WO 2021/139678, WO 2021/139748, CN 111205286, WO 2021/143693, CN 113135924, WO 2021/147965, WO 2021/155716, WO 2021/168193, WO 2021/169990, WO 2021/169963, CN 113321654, WO 2021/175199, WO 2021/180181, WO 2021/185233, WO 2021/190467, WO 2021/197499, CN 112574199 and CN 112300269, the contents of which are incorporated herein by reference in their entireties.

In some embodiments, provided is a method of treating a cancer in an individual (e.g., a human), comprising administering to the individual (a) an effective amount of a composition comprising nanoparticles comprising an mTOR inhibitor and an albumin (an “mTOR inhibitor nanoparticle composition; and (b) an effective amount of a KRAS G12C inhibitor, wherein the KRAS G12C inhibitor is sotorasib (also known as AMG 510, LUMAKRAS™, and LUMYKRAS™), adagrasib (also known as MRTX849), JAB-21822 (also known as JAB-21000), GDC-6036, JDQ443, D-1553, GH35, GFH925, BPI-421286, LY3537982, RMC-6291, RMC-8839, HBI-2438, and JNJ-74699157. In some embodiments, the cancer comprises one or more cancer cells that express a KRAS G12C mutant protein. Additionally or alternatively, in some embodiments, the cancer comprises one or more cells that have at least one mTOR-activating aberration. In some embodiments, cancer is solid tumor, lung cancer, bladder cancer, appendiceal cancer, colorectal cancer, small bowel cancer, pancreatic cancer, or tumor of unknown origin. In some embodiments, the cancer or tumor (such as any of the preceding cancers or tumors) is advanced, unresectable, and/or metastatic. In some embodiments, the cancer is solid tumor (e.g., advanced, unresectable, and/or metastatic solid tumor), lung cancer (e.g., advanced, unresectable, and/or metastatic lung cancer), or bladder cancer (e.g., advanced, unresectable, and/or metastatic bladder cancer). In some embodiments, the lung cancer is non-small cell lung cancer (NSCLC), e.g., advanced, unresectable, and/or metastatic NSCLC.

In some embodiments, the KRAS G12C inhibitor is sotorasib, which, as noted above, is also known as AMG 510, LUMAKRAS™, and LUMYKRAS™. AMG 510 is currently under development by Amgen/Beigene. Sotorasib can exist in either of two atropisomeric forms and one is more active than the other (see, e.g., https://cen(dot)acs(dot)org/pharmaceuticals/drug(dash)discovery/Amgen(dash)unveils(dash) KRas(dash)inhibitor(dash)human/97/i14). Sotorasib selectively forms an irreversible covalent bond to the sulfur atom in the cysteine residue that is present in the G12C mutated form of the KRAS protein, but not in the wild type form. The covalent binding of sotorasib to KRAS G12C locks the protein in its inactive GDP-bound conformation, thus inhibiting KRAS-dependent signal transduction. Sotorasib has the empirical formula C30HF2N6O3 and a molecular weight of 560.606 g/mol. Sotorasib is described chemically as 6-Fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and has the following chemical structure:

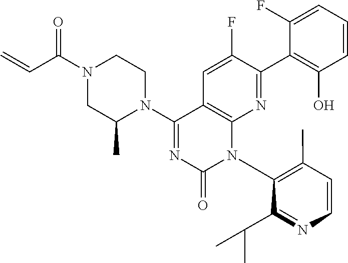

The CAS Registry Number for sotorasib is 2252403-56-6. The efficacy of sotorasib was demonstrated in a subset of patients enrolled in a single-arm, open-label, multicenter trial (NCT03600883) and is currently being investigated in further clinical trials. Complete information about sotorasib preparation, dispensing, dosage, and administration schedule can be found in the local package insert (for the United States, see, e.g., www(dot)accessdata(dot)fda(dot)gov/drugsatfda_docs/label/2021/214665s0001bl.pdf. Further details regarding the structure and synthesis of sotorasib are provided in WO 2018/217651, the contents of which are incorporated herein by reference in their entirety.

In some embodiments, the KRAS G12C inhibitor is adagrasib (also known as MRTX849). Adagrasib, which is currently under development by Mirati/Zai Lab. Like sotorasib, adagrasib selectively forms an irreversible covalent bond to the sulfur atom in the cysteine residue that is present in the G12C mutated form of the KRAS protein, but not in the wild type form. Like sotorasib, the covalent binding of adagrasib to KRAS G12C locks the protein in its inactive GDP-bound conformation, thus inhibiting KRAS-dependent signal transduction. Adagrasib has the empirical formula C32H35C1FN702 and a molecular weight of 604.13 g/mol. Adagrasib is described chemically as 2-[(2S)-4-[7-(8-chloronaphthalen-1-yl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrileand has the following chemical structure:

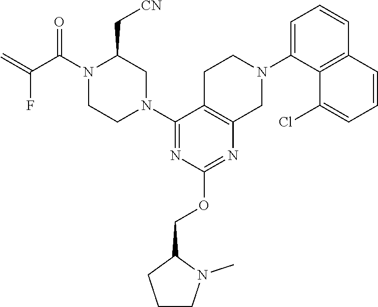

The CAS Registry Number for MRTX849 is 2326521-71-3. Adagrasib is currently being evaluated in several clinical trials, including NCT04613596, NCT04685135, NCT03785249, NCT04330664, and others. Further details regarding the structure and synthesis of adagrasib are provided in Fell et al. (2020) J. Med. Chem. 63, 6679-6693 and WO 2017/201161, the contents of which are incorporated herein by reference in their entirety.

In some embodiments, the KRAS G12C inhibitor is JAB-21822. JAB-21822 is being developed by Jacobio Pharmaceuticals Group Co., LTD. (see, e.g., en(dot)jacobiopharma(dot)com/blank3(dot)html?introld=23 and www(dot)jacobiopharma(dot)com/news/207(dot)html). JAB-21822 is currently being evaluated in several clinical trials, including NCT05009329 and NCT05002270. Further details regarding the structure and synthesis of JAB-21822 are provided in WO 2021/057832, the contents of which are incorporated herein by reference in their entirety.

In some embodiments, the KRAS G12C inhibitor is GDC-6036. GDC-6036 is being developed by Genentech, Inc. (see, e.g., www(dot)genentechoncology(dot)com/pipeline-molecules/kras-gl2c.html) and is currently being evaluated clinical trial NCT04449874. Further details regarding the structure and synthesis of GDC-6036 are provided in WO 2020/097537, the contents of which are incorporated herein by reference in their entirety.

In some embodiments, the KRAS G12C inhibitor is JDQ443. JDQ443 is an inhibitor of KRAS G12C that is being developed by Novartis. The structure of JDQ443 is:

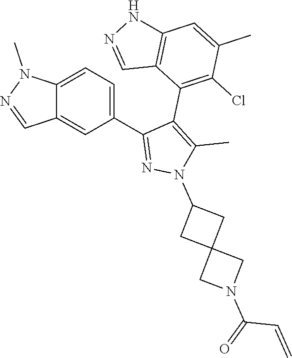 [SMILES: C=CC(=O)N1CC2(CC(n3nc(-c4ccc5c(cnn5C)c4)c(-c4c(Cl)c(C)cc5[nH]ncc45)c3C)C2)C1]

JDQ443 is currently being evaluated in clinical trial NCT04699188. Further details regarding the synthesis of JDQ443 are provided in WO 2021/124222, the contents of which are incorporated herein by reference in their entirety.

In some embodiments, the KRAS G12C inhibitor is D-1553. D-1553 is being developed by InventisBio Co., Ltd. (see, e.g., www(dot)inventisbio(dot)com/% e4% b8% b4% e5% ba %8a % e8% af %/o95% e9% aa %8c/) and is currently being evaluated in clinical trial NCT04585035 in collaboration with Merck Sharp & Dohme. Further details regarding the structure and synthesis of D-1553 are provided in WO 2020/233592, the contents of which are incorporated herein by reference in their entirety.

In some embodiments, the KRAS G12C inhibitor is GH35. GH35 is being developed by Suzhou Genhouse Bio Co., Ltd. (see, e.g., www(dot)genhousebio(dot)com/en/product/index(dot)html) and is being evaluated in clinical trial NCT05010694. Further details regarding the structure and synthesis of GH35 are provided in WO2020/177653, the contents of which are incorporated herein by reference in their entirety.

In some embodiments, the KRAS G12C inhibitor is GFH925. GFH925 is being developed by GenFleet Therapeutics (Zhejiang) (see, e.g., www(dot)genfleet(dot)com/en/science) and is being evaluated in clinical trial NCT05005234. Further details regarding the structure and synthesis of GFH925 are provided in WO 2020/177629, WO 2020/221239, and WO 2021/031952, the contents of which are incorporated herein by reference in their entirety.

In some embodiments, the KRAS G12C inhibitor is BPI-421286. BPI-421286 is being developed by Betta Pharmaceutical Co., Ltd (see, e.g., www(dot)bettapharma(dot)com/News/show/id/2380), and clinical trial applications for the evaluation of BPI-421286 (CXHL2100046 and CXHL2100047) have been accepted by the State Food and Drug Administration of the People's Republic of China. Further details regarding the structure and synthesis of BPI-421286 are provided in CN112390796, the contents of which are incorporated herein by reference in their entirety.

In some embodiments, the KRAS G12C inhibitor is LY3537982. LY3537982 is being developed by Eli Lilly and Company and Loxo Oncology, Inc. (see, e.g., www(dot)lillyloxooncologypipeline(dot)com/molecule/kras-gl2c-inhibitor/) and is being investigated in clinical trial NCT04956640. Further details regarding the structure and synthesis of LY3537982 are provided in WO2021/118877, the contents of which are incorporated herein by reference in their entirety.

In some embodiments, the KRAS G12C inhibitor is RMC-6291. RMC-6291 is currently under development by Revolution Medicines, Inc. (see, e.g., www(dot)revmed(dot)com/pipeline and is being investigated in clinical trial NCT05462717. In some embodiments, the KRAS G12C inhibitor is RMC-8839, which is also under development by Revolution medicines, Inc.

In some embodiments, the KRAS G12C inhibitor is HBI-2438, which is currently under development by HUYA Bioscience International, LLC. HBI-2438 is being investigated in clinical trial NCT05485974.

In some embodiments, the KRAS G12C inhibitor is JNJ-74699157 (also known as ARS-3248), which is under development by Johnson and Johnson and Wellspring Bioscience. JNJ-74699157 is being investigated in clinical trial NCT04006301.

As discussed above, in some embodiments, the KRAS G12C inhibitor and the mTOR inhibitor nanoparticle composition (such as sirolimus/albumin nanoparticle composition) are administered in a single unit dose. In some embodiments, the KRAS G12C inhibitor and the mTOR inhibitor nanoparticle composition (such as sirolimus/albumin nanoparticle composition) are administered in separate dosage forms.

### KRAS G12D Inhibitors

The KRAS G12D mutation incidence is the high in pancreatic cancers (e.g., pancreatic ductal adenocarcinoma), lung cancers, and colorectal cancers, with a strong correlation with poor prognosis. The G12D substitution also favors the activated GTP-bound state of KRAS, amplifying signaling pathways that lead to oncogenesis (see, e.g., Lee et al. (2021) Chem. Sci., 12, 12827-12837).

In some embodiments, the term “KRAS G12D inhibitor” refers to any agent, e.g., polypeptide, fusion polypeptide, antibody, peptide, antisense oligonucleotide, or small molecule drug, that inhibits the activity of the KRAS G12D mutant protein. In some embodiments, the KRAS G12D inhibitor interacts directly with the KRAS G12D mutant protein to inhibit the protein's activity. In some embodiments, the KRAS G12D inhibitor is a small molecule drug. Exemplary KRAS G12D inhibitors that find use with the methods provided herein include, without limitation, MRTX1133 (Mirati Therapeutics) and RMC-6236 (Revolution Medicines).

In some embodiments, the KRASG12D inhibitor is MRTX1133 (CAS Registry Number 2621928-55-8), which is under development by Mirati Therapeutics (see, e.g., world-wide-web(dot)mirati(dot)com/science/programs/kras-inhibitors/kras-g12d-inhibitor/. The structure of MRTX1133 is:

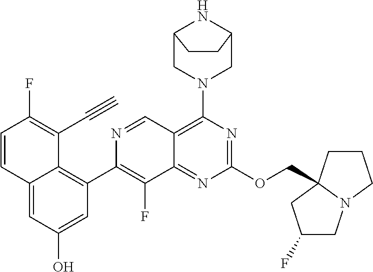 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

Further details regarding MRTX1133 are provided in Wang et al. (2022) J Med Chem 65(4):3123-3133, The KRASG12D inhibitor MRTX1133 elucidates KRAS-mediated oncogenesis. Nat Med (2022) (doi(dot)org/10(dot)1038/s41591-022-02008-6), and Hallin, Bowcut, Calinisan, et al. Anti-tumor efficacy of a potent and selective non-covalent KRAS^{G12D} inhibitor. Nat Med (2022) (doi(dot)org/10(dot)1038/s41591-022-02007-7), the contents of which are incorporated herein by reference in their entirety.

In some embodiments, the KRAS G12D inhibitor is RMC-9805, which is under development by Revolution Medicines (see, e.g., world-wide-web(dot)revmed(dot)com/pipeline/rason-inhibitors.

As discussed above, in some embodiments, the KRAS G12D inhibitor and the mTOR inhibitor nanoparticle composition (such as sirolimus/albumin nanoparticle composition) are administered in a single unit dose. In some embodiments, the KRAS G12D inhibitor and the mTOR inhibitor nanoparticle composition (such as sirolimus/albumin nanoparticle composition) are administered in separate dosage forms.

### KRAS G12V Inhibitors

The KRAS G12V mutation incidence is the high in pancreatic cancers (e.g., pancreatic ductal adenocarcinoma), lung cancers, and colorectal cancers. In some embodiments, the term “KRAS G12V inhibitor” refers to any agent, e.g., polypeptide, fusion polypeptide, antibody, peptide, antisense oligonucleotide, or small molecule drug, that inhibits the activity of the KRAS G12V mutant protein. In some embodiments, the KRAS G12D inhibitor interacts directly with the KRAS G12V mutant protein to inhibit the protein's activity. In some embodiments, the KRAS G12V inhibitor is a small molecule drug. Exemplary KRAS G12V inhibitors that find use with the methods provided herein include, without limitation, JAB-23000 (Jacobio Pharmaceuticals). JAB-23000 is under development by Jacobiopharma. Further details regarding JAB-23000 are provided in Reck et al. (2001) Annals of Oncology. 32(9): 1101-1110 and entzz(dot)jacobiopharma(dot)com/upload/202108/20210831215444372(dot)pdf, the contents of which are incorporated by reference herein in their entireties.

As discussed above, in some embodiments, the KRAS G12V inhibitor and the mTOR inhibitor nanoparticle composition (such as sirolimus/albumin nanoparticle composition) are administered in a single unit dose. In some embodiments, the KRAS G12V inhibitor and the mTOR inhibitor nanoparticle composition (such as sirolimus/albumin nanoparticle composition) are administered in separate dosage forms.

Selection of Individuals for Treatment According to the Methods Herein mTOR-Activating Aberrations

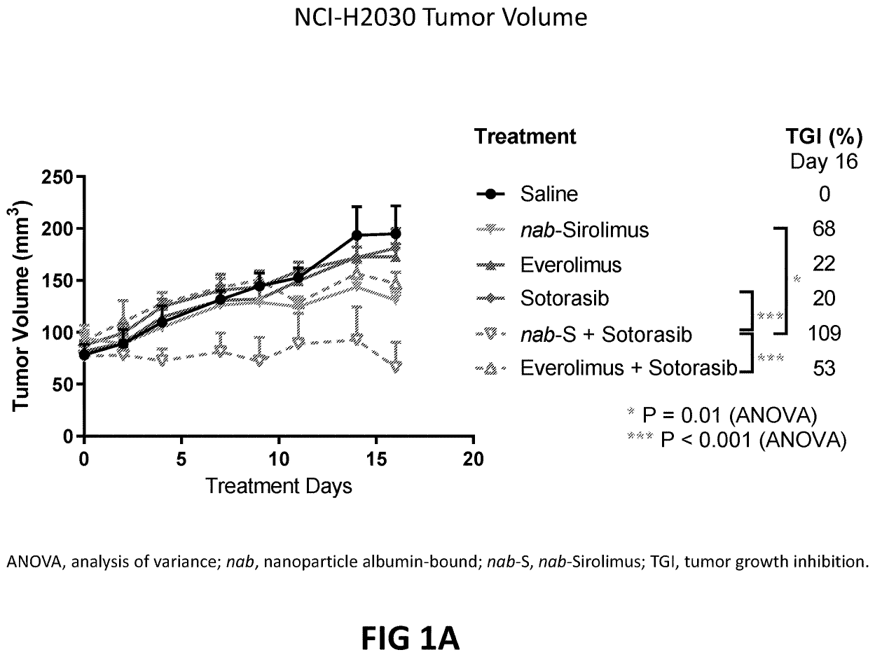

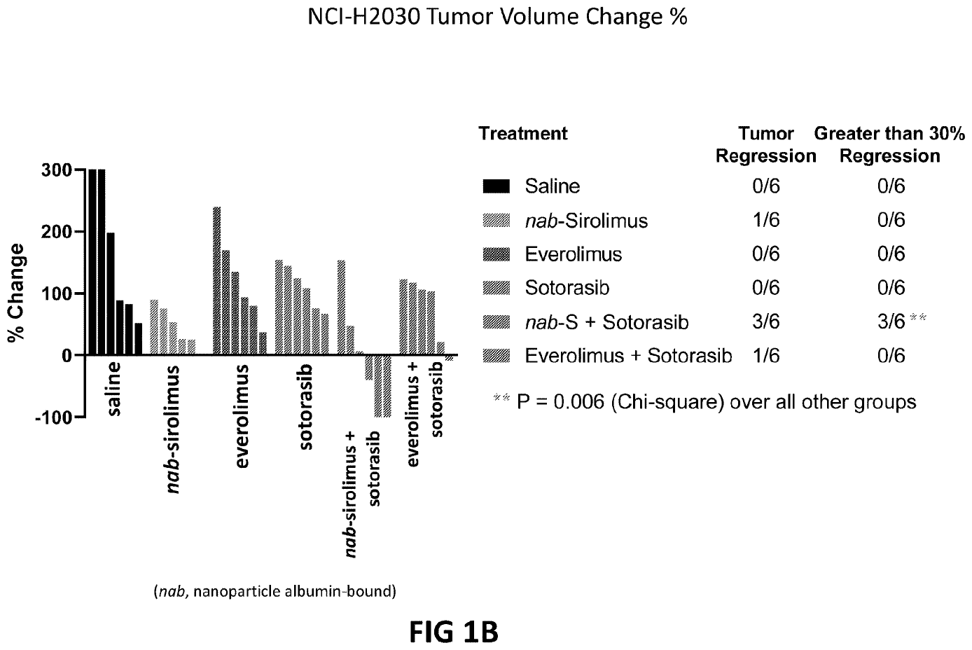

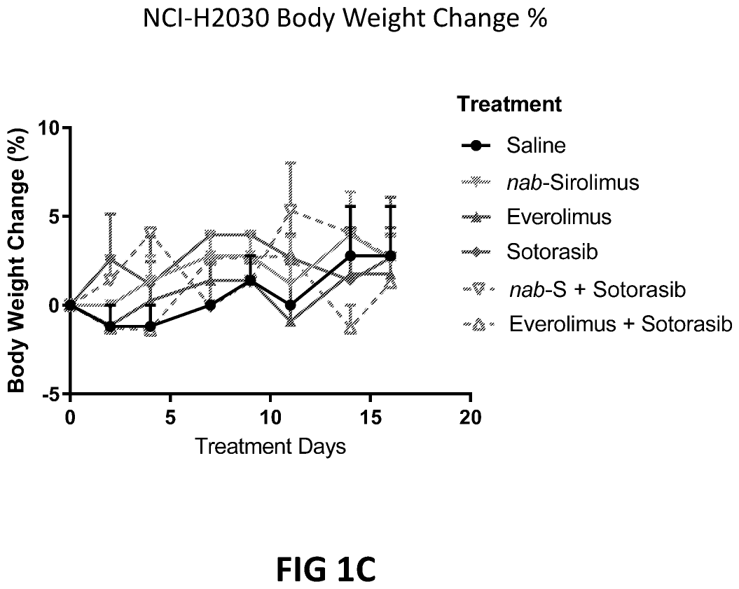

As discussed above, in some embodiments, the methods herein comprise (such as further comprise) selecting the individual for treatment based on the presence of one or more cancer cells with at least one mTOR-activating aberration in a sample from the individual prior to the administration of the mTOR inhibitor nanoparticle composition and the KRAS G12C inhibitor. In some embodiments, “mTOR-activating aberration” refers to a genetic aberration, an aberrant expression level and/or an aberrant activity level of one or more mTOR-associated gene that may lead to hyperactivation of the mTOR signaling pathway. In some embodiments, “hyperactivate” refers to increase of an activity level of a molecule (such as a protein or protein complex) or a signaling pathway (such as the mTOR a signaling pathway) to a level that is above a reference activity level or range, such as at least about any of 10%, 20%, 30%, 40%, 60%, 70%, 80%, 90%, 100%, 200%, 500% or more above the reference activity level or the median of the reference activity range. In some embodiments, the reference activity level is a clinically accepted normal activity level in a standardized test, or an activity level in a healthy individual (or tissue or cell isolated from the individual) free of the mTOR-activating aberration. In some embodiments, the at least one mTOR-activating aberration is an aberration associated with one or more mTOR-associated genes (e.g., AKT1, FLT-3, MTOR, PIK3CA, PIK3CG, TSC1, TSC2, RHEB, STK11, NF1, NF2, TP53, FGFR4, BAP1, KRAS, NRAS, NRF2, KEAP1, and PTEN) including deviations from the reference sequences (i.e. genetic aberrations), abnormal expression levels and/or abnormal activity levels of the one or more proteins encoded by mTOR-associated genes. In some embodiments, the at least one mTOR-activating aberration is an aberration associated with TSC1 and/or TSC2. In some embodiments, the at least one mTOR-activating aberration comprises a STK11 and/or TP53 aberration. Exemplary STK11 aberrations include STK11 E317, SKT11 null, and any of the mutations in FIG. 1 of Oncologist. 2020 Sep. 25(9): 733-737, which is incorporated in its entirety by reference. Exemplary TP53 aberrations include G262 (e.g., G262V), C176 (e.g., C176F), F113 (e.g., F113C) and any of the mutations described in FIG. 1 of Front Oncol. 2020; 10: 593383, which is incorporated in its entirety by reference. These exemplary TP53 mutations include loss of function mutation (e.g., R175H, G245S, R248Q, R248W, S241F, R249S, R273C, R273H, C275Y, R280K), partial function and/or temperature sensitive mutations (e.g., A161T, R181L, R202S, Y220H, S215C, D228V, V272L, R282W), wild type-like or super transactivating mutations (e.g., T123A, G199H, S240N, S288K, R337H, G360V), altered specificity (K120R, S121F, V122A, T125R, G279E), dominant mutations (e.g., R175H, G245S, R248Q, R248W, S241F, R249S, R273C, R273H, C275Y, R280K), and D281G, R282W. In some embodiments, the mTOR inhibitor aberration comprises a PTEN aberration (e.g., PTEN null) and/or PIK3CA aberration (e.g., PIK3CA mutant). In some embodiments, the individual further comprises an ATM (e.g. Q2800fs), CDKN2A (e.g., CDKN2A null) and/or UGT2B17 aberration (e.g., UGT2B17 null).

In some embodiments, the at least one mTOR-activating aberration is a genetic aberration. Genetic aberrations of one or more mTOR-associated genes may comprise a change to the nucleic acid (such as DNA and RNA) or protein sequence (i.e. mutation) or an epigenetic feature associated with an mTOR-associated gene described herein, including, but not limited to, coding, non-coding, regulatory, enhancer, silencer, promoter, intron, exon, and untranslated regions of the mTOR-associated gene.

In some embodiments, the genetic aberration may be a germline mutation (including chromosomal rearrangement), or a somatic mutation (including chromosomal rearrangement). In some embodiments, the genetic aberration is present in all tissues, including normal tissue and the cancer tissue, of the individual. In some embodiments, the genetic aberration is present only in the cancer tissue of the individual. In some embodiments, the genetic aberration is present only in a fraction of the cancer tissue.

In some embodiments, the mTOR-activating aberration comprises a mutation of an mTOR-associated gene described herein, including, but not limited to, deletion, frameshift, insertion, indel, missense mutation, nonsense mutation, point mutation, single nucleotide variation (SNV), silent mutation, splice site mutation, splice variant, and translocation. In some embodiments, the mutation may be a loss of function mutation for a negative regulator of the mTOR signaling pathway or a gain of function mutation of a positive regulator of the mTOR signaling pathway. In some embodiments, the mutation of an mTOR-associated gene is a mutation of TSC1 or TSC2.

In some embodiments, the genetic aberration comprises a copy number variation of an mTOR-associated gene described herein. Normally, there are two copies of each mTOR-associated gene per genome. In some embodiments, the copy number of the mTOR-associated gene is amplified by the genetic aberration, resulting in at least about any of 3, 4, 5, 6, 7, 8, or more copies of the mTOR-associated gene in the genome. In some embodiments, the genetic aberration of the mTOR-associated gene results in loss of one or both copies of the mTOR-associated gene in the genome. In some embodiments, the copy number variation of the mTOR-associated gene is loss of heterozygosity of the mTOR-associated gene. In some embodiments, the copy number variation of the mTOR-associated gene is deletion of the mTOR-associated gene. In some embodiments, the copy number variation of the mTOR-associated gene is caused by structural rearrangement of the genome, including deletions, duplications, inversion, and translocation of a chromosome or a fragment thereof. In some embodiments, the copy number variation of an mTOR-associated gene is a copy number variation of TSC1 or TSC2.

In some embodiments, the genetic aberration comprises an aberrant epigenetic feature associated with an mTOR-associated gene described herein, including, but not limited to, DNA methylation, hydroxymethylation, aberrant histone binding, chromatin remodeling, and the like. In some embodiments, the promotor of the mTOR-associated gene is hypermethylated in the individual, for example by at least about any of 10%, 20%, 30%, 40%, 50%, 60%, 70%, 80%, 90%, or more compared to a control level (such as a clinically accepted normal level in a standardized test). In some embodiments, the aberrant epigenetic feature that is associated with an mTOR-associated gene is associated with TSC1 or TSC2.

The genetic aberration(s) of mTOR-associated gene(s) described herein (e.g., TSC1 or TSC2) may be assessed based on a sample, such as a sample from the individual and/or reference sample. In some embodiments, the sample is a tissue sample or nucleic acids extracted from a tissue sample (e.g., a tumor tissue sample). In some embodiments, the sample is a cell sample or nucleic acids extracted from a cell sample (e.g., a CTC sample). In some embodiments, the sample is a tumor biopsy. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample. In some embodiments, the sample is a blood sample. In some embodiments, cell-free DNA is isolated from the blood sample. In some embodiments, the biological sample is a plasma sample or nucleic acids extracted from the plasma sample.

The genetic aberrations of the mTOR-associated gene may be determined by any method known in the art. See, for example, Dickson et al. Int. J Cancer, 2013, 132(7): 1711-1717; Wagle N. Cancer Discovery, 2014, 4:546-553; and Cancer Genome Atlas Research Network. Nature 2013, 499: 43-49. Exemplary methods include, but are not limited to, genomic DNA sequencing, bisulfite sequencing or other DNA sequencing-based methods using Sanger sequencing or next generation sequencing platforms; polymerase chain reaction assays; in situ hybridization assays; and DNA microarrays. The epigenetic features (such as DNA methylation, histone binding, or chromatin modifications) of one or more mTOR-associated genes from a sample isolated from the individual may be compared with the epigenetic features of the one or more mTOR-associated genes from a control sample. The nucleic acid molecules extracted from the sample can be sequenced or analyzed for the presence of the mTOR-activating genetic aberrations relative to a reference sequence, such as the wild type sequences of AKT1, FLT-3, MTOR, PIK3CA, PIK3CG, TSC1, TSC2, RHEB, STK11, NF1, NF2, TP53, FGFR4, BAP1, KRAS, NRAS, NRF2, KEAP1, and PTEN.

In some embodiments, the genetic aberration of an mTOR-associated gene is assessed using cell-free DNA sequencing methods. In some embodiments, the genetic aberration of an mTOR-associated gene is assessed using next-generation sequencing. In some embodiments, the genetic aberration of an mTOR-associated gene isolated from a blood sample is assessed using next-generation sequencing. In some embodiments, the genetic aberration of an mTOR-associated gene is assessed using exome sequencing. In some embodiments, the genetic aberration of an mTOR-associated gene is assessed using fluorescence in-situ hybridization analysis. In some embodiments, the genetic aberration of an mTOR-associated gene is assessed prior to initiation of the methods of treatment described herein. In some embodiments, the genetic aberration of an mTOR-associated gene is assessed after initiation of the methods of treatment described herein. In some embodiments, the genetic aberration of an mTOR-associated gene is assessed prior to and after initiation of the methods of treatment described herein.

Genetic mTOR-Activating Aberrations in TSC1 and TSC2

In some embodiments, the genetic mTOR-activating aberration comprises an in-frame deletion mutation in TSC1 or TSC2. In some embodiments, the in-frame deletion mutation has been reported in the Leiden Open Variation Database (“LOVD,” available at, e.g., databases(dot)lovd(dot)nl/shared/genes/TSC2). In some embodiments, the in-frame deletion mutation in TSC1 or TSC2 deletes a size of more than one amino acids. In some embodiments, the genetic mTOR-activating aberration comprises a missense mutation in TSC1. In some embodiments, the missense mutation in TSC1 comprises a non-conservative substitution within amino acids 34-224 or exons 4-8 of TSC1. In some embodiments, the genetic mTOR-activating aberration comprises a missense mutation in TSC2. In some embodiments, the missense mutation in TSC2 comprises a non-conservative substitution and/or has been reported in the LOVD database. In some embodiments, the genetic mTOR-activating aberration comprises a homozygous deletion mutation. In some embodiments, the homozygous deletion mutation affects one or more exons of TSC1 or TSC2.

Details regarding methods of assessing whether mutation(s) in TSC1 or TSC2 are genetic mTOR-activating aberrations (e.g., “pathogenic mutations”) are provided in PCT/US2020/060070, the contents of which are incorporated herein in their entirety.

### TSC2

TSC2 is also known as Tuberin, Tuberous sclerosis 2 protein, protein phosphatase 1 regulatory subunit 160, TSC4, PPP1R160, and LAM. TSC2 protein functions as part of a complex with TSC1 by negatively regulating mTORC1 signaling. In some embodiments, the nucleic acid sequence of a wild type TSC2 gene is identified by the Genbank accession number NC_000016.10, from nucleotide 2047936 to nucleotide 2088712 on the forward strand of chromosome 16 according to the GRCh38.p2 assembly of the human genome. The wild type TSC2 gene comprises 42 exons. A mutation of the TSC2 gene may occur in any one or any combination of the 42 exons, or in any intron or noncoding regions of the TSC2 gene.

In some embodiments, the amino acid sequence of a wild type TSC2 protein is identified by the Genbank accession number NP_000539.2. In some embodiments, the amino acid sequence of a wild type TSC2 protein is identified by the Genbank accession number NP_001070651.1. In some embodiments, the amino acid sequence of a wild type TSC2 protein is identified by the Genbank accession number NP_001107854.1.

In some embodiments, the nucleic acid sequence of a cDNA encoding a wild type TSC2 protein is identified by the Genbank accession number NM_000548.3. In some embodiments, the nucleic acid sequence of a cDNA encoding a wild type TSC2 protein is identified by the Genbank accession number NM_001077183.1. In some embodiments, the nucleic acid sequence of a cDNA encoding a wild type TSC2 protein is identified by the Genbank accession number NM_001114382.1.

In some embodiments, the individual is selected for treatment based on having an mTOR-activating aberration at TSC2. In some embodiments, the mTOR-activating aberration at TSC2 comprises a mutation (e.g., inactivating mutation) in TSC2. In some embodiments, the mutation is selected from the group consisting of a splice site mutation, a nonsense mutation, a frameshift mutation, a missense mutation, and a loss or deletion of the gene. In some embodiments, the mTOR-activating aberration at TSC2 comprises a single-nucleotide variant (SNV). In some embodiments, the SNV comprises a mutation selected from the group consisting of C1503T, C2743G, C5383T, C3755G, G760T, C3442T, G880A, T707C, A4949G, or a deletion of any one or more of the amino acids at the position of 1405-1409, 1960-1970, 4999, 5002, 3521, 5208, 5238-5255.

In some embodiments, the mutation is a two-point mutation (i.e., bi-allelic mutations). In some embodiments, the mutation comprises three-point mutation or four-point mutation. In some embodiments, the mTOR-activating aberration at TSC2 is a loss of function mutation. In some embodiments, the mTOR-activating aberration at TSC2 comprises a homozygous deletion. In some embodiments, the mTOR-activating aberration at TSC2 comprises a copy number variation of TSC2. In some embodiments, the mTOR-activating aberration at TSC2 comprises an aberrant expression level of TSC2. In some embodiments, the mTOR-activating aberration at TSC2 comprises an aberrant activity level of a protein encoded by TSC2.

In some embodiments, the individual has a mutation (e.g., inactivating mutation) in any one or more of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, and 44 according to Genbank accession number NM_000548. In some embodiments, the individual has bi-allelic mutations (e.g., bi-allelic inactivating mutation) in two of exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, and 44 according to Genbank accession number NM_000548. In some embodiments, the individual has an inactivating mutation in any of exons 18, 22, 27, 30, and 42 of TSC2. In some embodiments, the individual has bi-allelic mutations in any two of exons 18, 22, 27, 30, and 42 of TSC2. In some embodiments, the individual has bi-allelic mutations in exons 18 and 30 of TSC2. In some embodiments, the individual has bi-allelic mutations in exons 22 and 27 of TSC2.

In some embodiments, the mutation is not within amino acids 947-989 or exon 26. In some embodiments, the mutation is not within amino acids 1272-1295 or exon 32.

In some embodiments, the mutation comprises a non-conservative substitution.

In some embodiments, the mutation has been reported by the LOVD database.

TSC1 and TSC2 gene mutations were described in e.g., Rosset et al., Genetics and Molecular Biology, 40, 1, 69-79 (2017), which is incorporated herein by its entirety. In some embodiments, the individual has a continuous deletion (e.g., TSC2-PKD1 deletion). See e.g., Boronat et al., Brain Dev. 36:801-806. In some embodiments, the individual has a c.5238-5255 del in TSC2. See e.g., Rok et al. Med Sci Monit 11:230-234. In some embodiments, the individual has a proximal region mutation (e.g., in any of exons 1-22) and/or a distal region mutation (e.g., in any of exons 23-41). See e.g., van Eeghena et al. Epilepsy Res 103:83-87.

### TSC1

TSC1 is also known as Hamartin, Tuberous sclerosis 1 protein, TSC, KIAA0243, and LAM. TSC1 protein functions as part of a complex with TSC2 by negatively regulating mTORC1 signaling. In some embodiments, the nucleic acid sequence of a wild type TSC1 gene is identified by the Genbank accession number NC_000009.12, from nucleotide 132891348 to nucleotide 132945370 on the reverse strand of chromosome 9 according to the GRCh38.p2 assembly of the human genome. The wild type TSC1 gene comprises 25 exons. A mutation of the TSC1 gene may occur in any one or any combination of the 25 exons, or in any intron or noncoding regions of the TSC1 gene.

In some embodiments, the amino acid sequence of a wild type TSC1 protein is identified by the Genbank accession number NP_000359.1. In some embodiments, the amino acid sequence of a wild type TSC1 protein is identified by the Genbank accession number NP_001155898.1. In some embodiments, the amino acid sequence of a wild type TSC1 protein is identified by the Genbank accession number NP_001155899.1.

In some embodiments, the nucleic acid sequence of a cDNA encoding a wild type TSC1 protein is identified by the Genbank accession number NM_000368.4. In some embodiments, the nucleic acid sequence of a cDNA encoding a wild type TSC1 protein is identified by the Genbank accession number NM_001162426.1. In some embodiments, the nucleic acid sequence of a cDNA encoding a wild type TSC1 protein is identified by the Genbank accession number NM_001162427.1.

In some embodiments, the individual is selected for treatment on the basis of having an mTOR-activating aberration at TSC1. In some embodiments, the mTOR-activating aberration at TSC1 comprises a mutation (e.g., an inactivating mutation) in TSC1. In some embodiments, the mutation is selected from the group consisting of a splice site mutation, a nonsense mutation, a frameshift mutation, a missense mutation and a loss or deletion of the gene. In some embodiments, the mTOR-activating aberration at TSC1 comprises a single-nucleotide variant (SNV). In some embodiments, the mutation is a two-point mutation. In some embodiments, the mTOR-activating aberration at TSC1 is a loss of function mutation. In some embodiments, the mTOR-activating aberration at TSC1 comprises a homozygous deletion. In some embodiments, the mTOR-activating aberration at TSC1 comprises a copy number variation of TSC1. In some embodiments, the mTOR-activating aberration at TSC1 comprises an aberrant expression level of TSC1. In some embodiments, the mTOR-activating aberration at TSC1 comprises an aberrant activity level of a protein encoded by TSC1.

In some embodiments, the individual has a mutation (e.g., inactivating mutation) in any one or more of exon 1, 2, 3, 4, 5, 6, 7, 8, 9, 10, 11, 12, 13, 14, 15, 16, 17, 18, 19, 20, 21, 22, 23, 24, and 25 according to Genbank accession number NM_000368. In some embodiments, the individual has bi-allelic mutations (e.g., bi-allelic inactivating mutation) in two of exon 1, 2, 3, 4, 5, 6, 7, 8, 9, 10, 11, 12, 13, 14, 15, 16, 17, 18, 19, 20, 21, 22, 23, 24, and 25 according to Genbank accession number NM_000368. In some embodiments, the mutation is not in exon 23. In some embodiments, the mutation is not in 3′ half of exon 22.

In some embodiments, the mutation comprises a non-conservative substitution.

In some embodiments, the mutation has been reported by the LOVD database.

In some embodiments, the individual has a TSC1 loss or deletion.

### KRAS Mutations

In some embodiments, the methods described herein comprise (such as further comprises) selecting the individual for treatment based on the presence of one or more cancer cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS GT3V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K1T17N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein) in a sample from the individual prior to the administration of the mTOR inhibitor nanoparticle composition and the KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS GT3V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor, respectively). In some embodiments, cancer cells that express a KRAS G12C mutant protein comprise a KRAS G12C mutation.

A KRAS mutation may be assessed based on a sample, such as a sample from the individual and/or reference sample. In some embodiments, the sample is a tissue sample or nucleic acids extracted from a tissue sample (e.g., a tumor tissue sample). In some embodiments, the sample is a cell sample or nucleic acids extracted from a cell sample (e.g., a CTC sample). In some embodiments, the sample is a tumor biopsy. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample. In some embodiments, the sample is a blood sample. In some embodiments, cell-free DNA is isolated from the blood sample. In some embodiments, the biological sample is a plasma sample or nucleic acids extracted from the plasma sample.

In some embodiments, the KRAS mutation is assessed (such as detected) via nucleic acid sequencing (dideoxy and pyrosequencing), PCR (including allele-specific PCR and amplification refractory mutation system (ARMS) quantitative PCR, and digital PCR), single-strand conformational polymorphism analysis, melt-curve analysis, probe hybridization methods (including the use of nucleic acid and peptide nucleic acid probes). KRAS mutations (e.g., KRAS G12C mutations) can also be assessed (such as detected) using commercially available kits, e.g., THERASCREEN® assay (DxS, Manchester, UK), PYROMARK® KRAS assay (Qiagen, Valencia, CA, USA), SIGNATURE® KRAS/BRAF assay (Asuragen, Inc., Austin, TX, USA), and others. Such methods are described in, e.g., Matsunaga et al. (2016) Oncol Lett. 12(1): 150-156; Brychta et al. (2016) Clinical Chemistry, Volume 62, Issue 11: 1482-1491; Nicolazzo et al. (2021) Diagnostics (Basel). 11(12): 2196; and Anderson (2011) Expert Rev Mol Diagn. 11(6):635-642, as well as the references cited in each of the preceding. Exemplary platforms for screening patient samples for KRAS G12C mutation include polymerase chain reaction (PCR) for tumor tissue (e.g., Qiagen therascreen KRAS RGQ PCR Kit) and next-generation sequencing (NGS) for ctDNA (e.g., Resolution Bioscience.

### Other Selection Criteria

In some embodiments, the individual treated according to a method described herein has a histologically confirmed diagnosis of a solid tumor malignancy with KRAS mutation (e.g., KRAS G12C mutation) in tumor tissue or plasma ctDNA. In some embodiments, the solid tumor is advanced (e.g., in stage III or stage IV, or terminal stage), unresectable, and/or metastatic solid tumor. In some embodiments, the tumor is at least about 0.5, 1, 1.25, 1.5, 1.75, or 2 centimeter in diameter. In some embodiments, the individual treated according to a method described herein has a histologically confirmed diagnosis of NSCLC with KRAS mutation (e.g., KRAS G12C mutation) in tumor tissue or plasma ctDNA. In some embodiments, the NSCLC is advanced, unresectable, and/or metastatic NSCLC. In some embodiments, the individual is not a candidate for definitive therapy. In some embodiments, there is no available treatment with curative intent for the solid tumor malignancy and/or NSCLC. In some embodiments, the individual has received prior therapy with platinum compound and/or a checkpoint inhibitor (with any therapeutic intent). In some embodiments, the individual has not received a prior therapy with a KRAS inhibitor (e.g., KRAS G12C inhibitor). In some embodiments, the individual has received prior therapy with a KRAS inhibitor (e.g., KRAS G12C inhibitor). In some embodiments, the individual has measurable disease per RECIST 1.1 (see Eisenhauer et al. (2009) Eur J. Cancer 45: 228-247). In some embodiments, the individual is 18 years of age or older. In some embodiments, the individual has a life expectancy of at least 3 months. In some embodiments, wherein the individual received recent prior systemic therapy or radiation therapy, the recent prior systemic therapy (e.g., chemotherapy, immunotherapy, or an investigational agent) and radiation therapy was discontinued at least 2 weeks before first dose of the mTOR inhibitor nanoparticle composition and/or the KRAS inhibitor (e.g., KRAS G12C inhibitor). In some embodiments, wherein the individual has experienced adverse effects from prior therapy, the individual has recovered from the adverse effects of prior therapy at the time of enrollment to ≤Grade 1 (excluding alopecia, peripheral neuropathy, and parameters superseded by other eligibility criteria, such as hematology parameters). In some embodiments, the individual has an Eastern Cooperative Oncology Group (ECOG) performance status of 0 or 1 (see, e.g., www(dot)ecog-acrin(dot)org/resources/ecog-performance-status). In some embodiments, the individual has adequate organ function (including one or more of, e.g., absolute neutrophil count≥1,500/mm³ (1.5×10⁹/L); platelet count≥100,000/mm³ (100×10⁹/L); hemoglobin≥9 g/dL, in the absence of transfusions for at least 2 weeks; total bilirubin≤1.5× Upper Limit of Normal (ULN) (if associated with liver metastases or Gilbert's disease, ≤3×ULN); aspartate transaminase (AST) and alanine transaminase (ALT)≤3.0×ULN (if associated with liver metastases, ≤5×ULN); creatinine clearance ≥50 mL/min or glomerular filtration rate≥50 mL/min/1.73 m² calculated using a validated prediction equation (e.g., Cockcroft-Gault, Modification of Diet in Renal Disease (MDRD), or 24-hour urine CrCl).

In some embodiments, the individual does not have active brain metastases or carcinomatous meningitis. In some embodiments, the individual has brain metastases and (a) the brain metastases are adequately treated, (b) the individual is neurologically stable, and (c) steroid dosing≤10 mg daily prednisone (or equivalent), if the individual is receiving steroid treatment. In some embodiments, the individual does not have a history of significant hemoptysis or hemorrhage within 4 weeks of the first dose of the mTOR inhibitor nanoparticle composition and/or the KRAS inhibitor (e.g., KRAS G12C inhibitor). In some embodiments, the individual has not had major surgery within 4 weeks of first dose of treatment with the mTOR inhibitor nanoparticle composition and the KRAS inhibitor. In some embodiments, the individual does not have a history of intestinal disease, inflammatory bowel disease, major gastric surgery, or other gastrointestinal conditions (e.g., uncontrolled nausea, vomiting, malabsorption syndrome). In some embodiments, the individual does not have (or does not have a history of) any of the following cardiac abnormalities: (a) unstable angina pectoris or myocardial infarction; (b) congestive heart failure □ New Your Heart Association Class 3; (c) prolonged corrected QT (QTc)>480 milliseconds on ECG during screening period or medical or family history of congenital Long QT Syndrome; (d) symptomatic or uncontrolled atrial fibrillation or other clinically significant arrhythmia. In some embodiments, the individual does not have a history of stroke or transient ischemic attack, e.g., within the previous 6 months. In some embodiments, the individual does not have an ongoing need for a medication with a known risk of Torsades de Pointes or substrate of CYP3A with narrow therapeutic index; strong inhibitor or inducer of CYP3A and/or P-gp; strong inhibitor of breast cancer resistant protein (BCRP); and proton pump inhibitors that cannot be switched to alternative treatment prior to the first dose of treatment with the mTOR inhibitor nanoparticle composition and/or the KRAS inhibitor (e.g., KRAS G12C inhibitor). In some embodiments, the individual does not have known human immunodeficiency virus (HIV) infection or acute or chronic hepatitis B or C infection. In some embodiments, the individual has known human immunodeficiency virus (HIV) infection with no detectable viral load or acute or chronic hepatitis B or C infection with no detectable viral load. In some embodiments, the individual is not immunocompromised. In some embodiments, the individual does not have a history of interstitial lung disease or radiation pneumonitis requiring steroid treatment, or any evidence of clinically active interstitial lung disease or pneumonitis. In some embodiments, the individual does not have uncontrolled diabetes.

Exemplary Routes of Administration, Dosages, and Administration Frequencies Compositions Comprising Nanoparticles Comprising an mTOR Inhibitor and an Albumin

In some embodiments, the mTOR inhibitor nanoparticle composition (e.g., a sirolimus/albumin nanoparticle composition, such as FYARRO™) is administered subcutaneously. In some embodiments, the mTOR inhibitor nanoparticle composition (e.g., a sirolimus/albumin nanoparticle composition, such as FYARRO™) is administered intravenously. In some embodiments, the mTOR inhibitor nanoparticle composition (e.g., a sirolimus/albumin nanoparticle composition, such as FYARRO™) is administered at a dose between about 1 mg/m² and about 150 mg/m², between about 5 mg/m² and about 75 mg/m2, e.g., via intravenous infusion. In some embodiments, the mTOR inhibitor nanoparticle composition (e.g., a sirolimus/albumin nanoparticle composition, such as FYARRO™) is administered at a dose of about any one of 5, 7.5, 10, 15, 30, 56, 75 or 100 mg/m², e.g., via intravenous infusion. In some embodiments, the mTOR inhibitor nanoparticle composition (e.g., a sirolimus/albumin nanoparticle composition, such as FYARRO™) is administered to the individual having cancer in one or more 21-day cycles (e.g., three week cycles). In some embodiments, the mTOR inhibitor nanoparticle composition (e.g., a sirolimus/albumin nanoparticle composition, such as FYARRO™) is administered to the individual once during each 21-day cycle (e.g., three week cycle). In some embodiments, the mTOR inhibitor nanoparticle composition (e.g., a sirolimus/albumin nanoparticle composition, such as FYARRO™) is administered during Week 1, Week 2, or Week 3 during each 21-day cycle (e.g., three week cycle). In some embodiments, the mTOR inhibitor nanoparticle composition (e.g., a sirolimus/albumin nanoparticle composition, such as FYARRO™) is administered on Day 1, Day 8, or Day 15 of each 21-day cycle (e.g., three week cycle). In some embodiments, the mTOR inhibitor nanoparticle composition (e.g., a sirolimus/albumin nanoparticle composition, such as FYARRO™) is administered to the individual twice during each 21-day cycle (e.g., three week cycle). In some embodiments, the mTOR inhibitor nanoparticle composition (e.g., a sirolimus/albumin nanoparticle composition, such as FYARRO™) is administered during Week 1 and Week 2 during each 21-day cycle (e.g., three week cycle). In some embodiments, the mTOR inhibitor nanoparticle composition (e.g., a sirolimus/albumin nanoparticle composition, such as FYARRO™) is administered during Week 2 and Week 3 during each 21-day cycle (e.g., three week cycle). In some embodiments, the mTOR inhibitor nanoparticle composition (e.g., a sirolimus/albumin nanoparticle composition, such as FYARRO™) is administered during Week 1 and Week 3 during each 21-day cycle (e.g., three week cycle). In some embodiments, the mTOR inhibitor nanoparticle composition (e.g., a sirolimus/albumin nanoparticle composition, such as FYARRO™) is administered on Day 1 and Day 8 of each 21-day cycle (e.g., three week cycle). In some embodiments, the mTOR inhibitor nanoparticle composition (e.g., a sirolimus/albumin nanoparticle composition, such as FYARRO™) is administered on Day 1 and Day 15 of each 21-day cycle (e.g., three week cycle). In some embodiments, the mTOR inhibitor nanoparticle composition (e.g., a sirolimus/albumin nanoparticle composition, such as FYARRO™) is administered on Day 8 and Day 15 of each 21-day cycle (e.g., three week cycle). In some embodiments, the mTOR inhibitor nanoparticle composition (e.g., a sirolimus/albumin nanoparticle composition, such as FYARRO™) is administered to the individual three times during each 21-day cycle (e.g., three week cycle). In some embodiments, the mTOR inhibitor nanoparticle composition (e.g., a sirolimus/albumin nanoparticle composition, such as FYARRO™) is administered during Week 1, Week 2, and Week 3 during each 21-day cycle (e.g., three week cycle). In some embodiments, the mTOR inhibitor nanoparticle composition (e.g., a sirolimus/albumin nanoparticle composition, such as FYARRO™) is administered on Day 1, Day 8, and Day 15 of each 21-day cycle (e.g., three week cycle). In some embodiments, the dosage of the mTOR inhibitor nanoparticle composition (e.g., a sirolimus/albumin nanoparticle composition, such as FYARRO™) is modified (e.g., if the individual experiences one or more adverse effects). Details regarding dosage modification for FYARRO™ and circumstances under which dosage modifications are made are detailed at www(dot)accessdata(dot)fda(dot)gov/drugsatfda_docs/label/2021/2133121bl.pdf.

### KRAS Inhibitors

In some embodiments, the KRAS inhibitor (e.g., KRAS G12C inhibitor) is administered orally. In some embodiments the KRAS inhibitor (e.g., KRAS G12C inhibitor) is administered at a dose between about 100 mg and about 1200 mg, between about 200 mg and about 1150 mg, between about 300 mg and about 1000 mg, between about 400 mg and about 1000 mg, between about 400 mg and about 960 mg, or between about 400 mg and about 800 mg.

In some embodiments, the KRAS inhibitor is a KRAS G12C inhibitor. In some embodiments, the KRAS G12C inhibitor is sotorasib. In some embodiments, the sotorasib is administered once daily. In some embodiments, the sotorasib is administered (e.g., orally) at a dose between about 100 mg and about 1200 mg, between about 200 mg and about 1150 mg, or between about 300 mg and about 1000 mg. In some embodiments, the sotorasib is administered (e.g., orally) at a dose of about 960 mg. In some embodiments, the dosage of sotorasib is modified. Details regarding dosage modification for sotorasib and circumstances under which dosage modifications are made are detailed in www(dot)accessdata(dot)fda.gov/drugsatfda_docs/label/2021/214665s000lbl(dot)pdf. In some embodiments, the KRAS G12C inhibitor is adagrasib. In some embodiments, the adagrasib is administered twice daily (e.g., bis en die or “BID”). In some embodiments, the adagrasib is administered at a dose between about 100 mg and about 1200 mg, between about 200 mg and about 1150 mg, between about 300 mg and about 1000 mg, between about 400 mg and about 1000 mg, between about 400 mg and about 960 mg, or between about 400 mg and about 800 mg. In some embodiments, the adagrasib is administered (e.g., orally) at a dose of about any one of 100 mg, 150 mg, 200 mg, 300 mg, 400 mg, 500 mg, 600 mg, 700 mg, 800 mg, 900 mg, 1,000 mg, 1,100 mg and 1,200 mg.

### Exemplary Methods

In some embodiments, provided is a method of treating cancer in an individual (e.g, a human), comprising administering to the subject (a) an effective amount of a composition comprising nanoparticles comprising an mTOR inhibitor and an albumin (an “mTOR inhibitor nanoparticle composition”), wherein the mTOR inhibitor in the nanoparticles is associated (e.g., coated) with the albumin; and (b) an effective amount of a KRAS G12C inhibitor. In some embodiments, the cancer comprises one or more cells that express the KRAS G12C mutant protein. Additionally or alternatively, in some embodiments, the cancer comprises one or more cells that have at least one mTOR-activating aberration. In some embodiments, the cancer is lung cancer (e.g., NSCLC), bladder cancer, appendiceal cancer, colorectal cancer, small bowel cancer, pancreatic cancer, or tumor of unknown origin. In some embodiments, the cancer is NSCLC or bladder cancer. In some embodiments, the mTOR inhibitor nanoparticle composition and the KRAS G12C inhibitor are administered simultaneously. In some embodiments, the mTOR inhibitor nanoparticle composition and the KRAS G12C inhibitor are administered sequentially. In some embodiments mTOR inhibitor nanoparticle composition is administered prior to the KRAS G12C inhibitor. In some embodiments of the methods of treatment, the KRAS G12C inhibitor is administered prior to the mTOR inhibitor nanoparticle composition. In some embodiments, the mTOR inhibitor is an mTOR inhibitor described herein. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. In some embodiments, the albumin is human albumin, e.g., human serum albumin. In some embodiments, the KRAS G12C inhibitor is a small molecule inhibitor. In some embodiments, the KRAS G12C inhibitor is sotorasib, shown below:

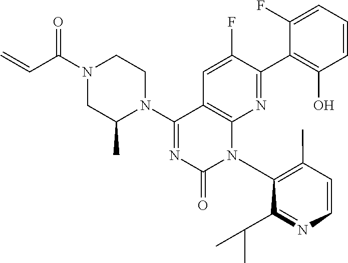

In some embodiments, the KRAS G12C inhibitor is adagrasib, shown below:

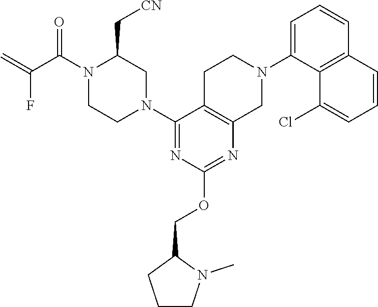

In some embodiments, the method comprises (such as further comprises) selecting the individual for treatment based on the presence of one or more cancer cells with at least one mTOR-activating aberration in a sample (e.g., tumor sample or blood sample) from the individual prior to the administration of the mTOR inhibitor nanoparticle composition and the KRAS G12C inhibitor. In some embodiments, the method comprises (such as further comprises) selecting the individual for treatment based on the presence of one or more cancer cells that express a KRAS G12C mutant protein in a sample (e.g., tumor sample or blood sample) from the individual prior to the administration of the mTOR inhibitor nanoparticle composition and the KRAS G12C inhibitor.

In some embodiments, provided is a method of treating cancer in an individual (e.g., a human), comprising administering to the subject (a) an effective amount of a composition comprising nanoparticles comprising an mTOR inhibitor and an albumin (an “mTOR inhibitor nanoparticle composition”), wherein the nanoparticles have an average particle size of no greater than about 150 nm (such as no greater than about 120 nm); and (b) an effective amount of a KRAS G12C inhibitor. In some embodiments, the cancer comprises one or more cells that express the KRAS G12C mutant protein. Additionally or alternatively, in some embodiments, the cancer comprises one or more cells that have at least one mTOR-activating aberration. In some embodiments, the cancer is lung cancer (e.g., NSCLC), bladder cancer, appendiceal cancer, colorectal cancer, small bowel cancer, pancreatic cancer, or tumor of unknown origin. In some embodiments, the cancer is NSCLC or bladder cancer. In some embodiments, the mTOR inhibitor nanoparticle composition and the KRAS G12C inhibitor are administered simultaneously. In some embodiments, the mTOR inhibitor nanoparticle composition and the KRAS G12C inhibitor are administered sequentially. In some embodiments mTOR inhibitor nanoparticle composition is administered prior to the KRAS G12C inhibitor. In some embodiments of the methods of treatment, the KRAS G12C inhibitor is administered prior to the mTOR inhibitor nanoparticle composition. In some embodiments, the mTOR inhibitor is an mTOR inhibitor described herein. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. In some embodiments, the albumin is human albumin, e.g., human serum albumin. In some embodiments, the KRAS G12C inhibitor is a small molecule inhibitor. In some embodiments, the KRAS G12C inhibitor is sotorasib, the structure of which is shown above. In some embodiments, the KRAS G12C inhibitor is adagrasib, the structure of which is shown above. In some embodiments, the method comprises (such as further comprises) selecting the individual for treatment based on the presence of one or more cancer cells with at least one mTOR-activating aberration in a sample (e.g., tumor sample or blood sample) from the individual prior to the administration of the mTOR inhibitor nanoparticle composition and the KRAS G12C inhibitor. In some embodiments, the method comprises (such as further comprises) selecting the individual for treatment based on the presence of one or more cancer cells that express a KRAS G12C mutant protein in a sample (e.g., tumor sample or blood sample) from the individual prior to the administration of the mTOR inhibitor nanoparticle composition and the KRAS G12C inhibitor.

In some embodiments, provided is a method of treating cancer in an individual (e.g., a human), comprising administering to the subject (a) an effective amount of a composition comprising nanoparticles comprising an mTOR inhibitor and an albumin (an “mTOR inhibitor nanoparticle composition”), wherein the nanoparticles comprise the mTOR inhibitor associated (e.g., coated) with albumin, and wherein the nanoparticles have an average particle size of no greater than about 150 nm (such as no greater than about 120 nm); and (b) an effective amount of a KRAS G12C inhibitor. In some embodiments, the cancer comprises one or more cells that express the KRAS G12C mutant protein. Additionally or alternatively, in some embodiments, the cancer comprises one or more cells that have at least one mTOR-activating aberration. In some embodiments, the cancer is lung cancer (e.g., NSCLC), bladder cancer, appendiceal cancer, colorectal cancer, small bowel cancer, pancreatic cancer, or tumor of unknown origin. In some embodiments, the cancer is NSCLC or bladder cancer. In some embodiments, the mTOR inhibitor nanoparticle composition and the KRAS G12C inhibitor are administered simultaneously. In some embodiments, the mTOR inhibitor nanoparticle composition and the KRAS G12C inhibitor are administered sequentially. In some embodiments mTOR inhibitor nanoparticle composition is administered prior to the KRAS GT2C inhibitor. In some embodiments of the methods of treatment, the KRAS G12C inhibitor is administered prior to the mTOR inhibitor nanoparticle composition. In some embodiments, the mTOR inhibitor is an mTOR inhibitor described herein. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. In some embodiments, the albumin is human albumin, e.g., human serum albumin. In some embodiments, the KRAS G12C inhibitor is a small molecule inhibitor. In some embodiments, the KRAS G12C inhibitor is sotorasib, the structure of which is shown above. In some embodiments, the KRAS G12C inhibitor is adagrasib, the structure of which is shown above. In some embodiments, the method comprises (such as further comprises) selecting the individual for treatment based on the presence of one or more cancer cells with at least one mTOR-activating aberration in a sample (e.g., tumor sample or blood sample) from the individual prior to the administration of the mTOR inhibitor nanoparticle composition and the KRAS G12C inhibitor. In some embodiments, the method comprises (such as further comprises) selecting the individual for treatment based on the presence of one or more cancer cells that express a KRAS G12C mutant protein in a sample (e.g., tumor sample or blood sample) from the individual prior to the administration of the mTOR inhibitor nanoparticle composition and the KRAS G12C inhibitor.

In some embodiments, provided is a method of treating cancer in an individual (e.g., a human), comprising administering to the subject (a) an effective amount of a composition comprising nanoparticles comprising an mTOR inhibitor and an albumin (an “mTOR inhibitor nanoparticle composition”), wherein the weight ratio of albumin and the mTOR inhibitor in the mTOR inhibitor nanoparticle composition is about 10:1 (such as about 10:1 or about 9:1 or about 8:1); and (b) an effective amount of a KRAS G12C inhibitor. In some embodiments, the cancer comprises one or more cells that express the KRAS G12C mutant protein. Additionally or alternatively, in some embodiments, the cancer comprises one or more cells that have at least one mTOR-activating aberration. In some embodiments, the cancer is lung cancer (e.g., NSCLC), bladder cancer, appendiceal cancer, colorectal cancer, small bowel cancer, pancreatic cancer, or tumor of unknown origin. In some embodiments, the cancer is NSCLC or bladder cancer. In some embodiments, the mTOR inhibitor nanoparticle composition and the KRAS G12C inhibitor are administered simultaneously. In some embodiments, the mTOR inhibitor nanoparticle composition and the KRAS G12C inhibitor are administered sequentially. In some embodiments mTOR inhibitor nanoparticle composition is administered prior to the KRAS G12C inhibitor. In some embodiments of the methods of treatment, the KRAS G12C inhibitor is administered prior to the mTOR inhibitor nanoparticle composition. In some embodiments, the mTOR inhibitor is an mTOR inhibitor described herein. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. In some embodiments, the albumin is human albumin, e.g., human serum albumin. In some embodiments, the KRAS G12C inhibitor is a small molecule inhibitor. In some embodiments, the KRAS G12C inhibitor is sotorasib, the structure of which is shown above. In some embodiments, the KRAS G12C inhibitor is adagrasib, the structure of which is shown above. In some embodiments, the method comprises (such as further comprises) selecting the individual for treatment based on the presence of one or more cancer cells with at least one mTOR-activating aberration in a sample (e.g., tumor sample or blood sample) from the individual prior to the administration of the mTOR inhibitor nanoparticle composition and the KRAS G12C inhibitor. In some embodiments, the method comprises (such as further comprises) selecting the individual for treatment based on the presence of one or more cancer cells that express a KRAS G12C mutant protein in a sample (e.g., tumor sample or blood sample) from the individual prior to the administration of the mTOR inhibitor nanoparticle composition and the KRAS G12C inhibitor.

In some embodiments, provided is a method of treating cancer in an individual (e.g., a human), comprising administering to the subject (a) an effective amount of a composition comprising nanoparticles comprising an mTOR inhibitor and an albumin (an “mTOR inhibitor nanoparticle composition”), wherein the nanoparticles comprise the mTOR inhibitor associated (e.g., coated) with albumin, wherein the nanoparticles have an average particle size of no greater than about 150 nm (such as no greater than about 120 nm), and wherein the weight ratio of albumin and the mTOR inhibitor in the mTOR inhibitor nanoparticle composition is about 10:1 (such as about 10:1 or about 9:1 or about 8:1); and (b) an effective amount of a KRAS G12C inhibitor. In some embodiments, the cancer comprises one or more cells that express the KRAS G12C mutant protein. Additionally or alternatively, in some embodiments, the cancer comprises one or more cells that have at least one mTOR-activating aberration. In some embodiments, the cancer is lung cancer (e.g., NSCLC), bladder cancer, appendiceal cancer, colorectal cancer, small bowel cancer, pancreatic cancer, or tumor of unknown origin. In some embodiments, the cancer is NSCLC or bladder cancer. In some embodiments, the mTOR inhibitor nanoparticle composition and the KRAS G12C inhibitor are administered simultaneously. In some embodiments, the mTOR inhibitor nanoparticle composition and the KRAS G12C inhibitor are administered sequentially. In some embodiments mTOR inhibitor nanoparticle composition is administered prior to the KRAS G12C inhibitor. In some embodiments of the methods of treatment, the KRAS G12C inhibitor is administered prior to the mTOR inhibitor nanoparticle composition. In some embodiments, the mTOR inhibitor is an mTOR inhibitor described herein. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. In some embodiments, the albumin is human albumin, e.g., human serum albumin. In some embodiments, the KRAS G12C inhibitor is a small molecule inhibitor. In some embodiments, the KRAS G12C inhibitor is sotorasib, the structure of which is shown above. In some embodiments, the KRAS G12C inhibitor is adagrasib, the structure of which is shown above. In some embodiments, the method comprises (such as further comprises) selecting the individual for treatment based on the presence of one or more cancer cells with in in a sample (e.g., tumor sample or blood sample) from the individual prior to the administration of the mTOR inhibitor nanoparticle composition and the KRAS G12C inhibitor. In some embodiments, the method comprises (such as further comprises) selecting the individual for treatment based on the presence of one or more cancer cells that express a KRAS G12C mutant protein in a sample (e.g., tumor sample or blood sample) from the individual prior to the administration of the mTOR inhibitor nanoparticle composition and the KRAS G12C inhibitor.

In some embodiments, provided is the use of a composition comprising nanoparticles comprising an mTOR inhibitor and an albumin (an “mTOR inhibitor nanoparticle composition”), wherein the nanoparticles comprise the mTOR inhibitor associated (e.g., coated) with albumin, wherein the nanoparticles have an average particle size of no greater than about 150 nm (such as no greater than about 120 nm), and/or wherein the weight ratio of albumin and the mTOR inhibitor in the mTOR inhibitor nanoparticle composition is about 10:1 (such as about 10:1 or about 9:1 or about 8:1), in the manufacture of a medicament for treating cancer in an individual (e.g., a human), wherein the medicament is for administration with a KRAS G12C inhibitor. In some embodiments, provided is the use of a KRAS G12C inhibitor in the manufacture of a medicament for treating cancer in an individual (e.g., a human), wherein the medicament is for administration with an mTOR inhibitor nanoparticle composition, wherein the nanoparticles comprise the mTOR inhibitor associated (e.g., coated) with albumin, wherein the nanoparticles have an average particle size of no greater than about 150 nm (such as no greater than about 120 nm), and/or wherein the weight ratio of albumin and the mTOR inhibitor in the mTOR inhibitor nanoparticle composition is about 10:1 (such as about 10:1 or about 9:1 or about 8:1). In some embodiments, the cancer comprises one or more cells that express the KRAS G12C mutant protein. Additionally or alternatively, in some embodiments, the cancer comprises one or more cells that have at least one mTOR-activating aberration. In some embodiments, the cancer is lung cancer (e.g., NSCLC), bladder cancer, appendiceal cancer, colorectal cancer, small bowel cancer, pancreatic cancer, or tumor of unknown origin. In some embodiments, the cancer is NSCLC or bladder cancer. In some embodiments, the mTOR inhibitor nanoparticle composition and the KRAS G12C inhibitor are administered simultaneously. In some embodiments, the mTOR inhibitor nanoparticle composition and the KRAS G12C inhibitor are administered sequentially. In some embodiments mTOR inhibitor nanoparticle composition is administered prior to the KRAS G12C inhibitor. In some embodiments of the methods of treatment, the KRAS G12C inhibitor is administered prior to the mTOR inhibitor nanoparticle composition. In some embodiments, the mTOR inhibitor is an mTOR inhibitor described herein. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. In some embodiments, the albumin is human albumin, e.g., human serum albumin. In some embodiments, the KRAS G12C inhibitor is a small molecule inhibitor. In some embodiments, the KRAS G12C inhibitor is sotorasib, the structure of which is shown above. In some embodiments, the KRAS G12C inhibitor is adagrasib, the structure of which is shown above. In some embodiments, the method comprises (such as further comprises) selecting the individual for treatment based on the presence of one or more cancer cells with at least one mTOR-activating aberration in a sample (e.g., tumor sample or blood sample) from the individual prior to the administration of the mTOR inhibitor nanoparticle composition and the KRAS G12C inhibitor. In some embodiments, the method comprises (such as further comprises) selecting the individual for treatment based on the presence of one or more cancer cells that express a KRAS G12C mutant protein in a sample (e.g., tumor sample or blood sample) from the individual prior to the administration of the mTOR inhibitor nanoparticle composition and the KRAS G12C inhibitor.

### Articles of Manufacture and Kits

Provided is an article of manufacture comprising materials useful for the treatment of a cancer in an individual. In certain embodiments, the article of manufacture or kit comprises a container containing composition comprising nanoparticles comprising an mTOR inhibitor and an albumin (an “mTOR inhibitor nanoparticle composition”). In some embodiments, the mTOR inhibitor is an mTOR inhibitor described herein. In some embodiments, the mTOR inhibitor is a limus drug. In some embodiments, the mTOR inhibitor is sirolimus (rapamycin) or a derivative or analog thereof. In some embodiments, the mTOR inhibitor nanoparticle composition comprises nab-sirolimus. In some embodiments, the mTOR inhibitor nanoparticle composition is nab-sirolimus. In some embodiments, the nanoparticles comprise the mTOR inhibitor associated (e.g., coated) with albumin. In some embodiments, the nanoparticles have an average particle size of no greater than about 150 nm (such as no greater than about 120 nm). In some embodiments, the weight ratio of albumin and the mTOR inhibitor in the mTOR inhibitor nanoparticle composition is about 10:1 or less (such as about 10:1 or about 9:1 or about 8:1). In some embodiments, the albumin is human albumin, e.g., human serum albumin. In some embodiments, the kit includes one or more positive controls, for example, cells having at least one mTOR-activating aberration (see elsewhere herein for further details regarding mTOR-activating aberrations). In some embodiments, the kit includes negative controls, for example a cell that does not have any mTOR-activating aberrations. In some embodiments, the kit is for treatment of a cancer that comprises one or more cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein). Additionally or alternatively, in some embodiments, the kit is for treatment of a cancer that comprises one or more cells that have at least one mTOR-activating aberration. In some embodiments, the cancer is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin. In some embodiments, the cancer is solid tumor, lung cancer, or bladder cancer. In some embodiments, the lung cancer is non-small cell lung cancer (NSCLC).

In certain embodiments, the article of manufacture or kit comprises a container and a label or package insert on or associated with the container. Suitable containers include, for example, bottles, vials, syringes, IV solution bags, test tubes, etc. The containers may be formed from a variety of materials such as glass or plastic. The container holds a composition (e.g., an mTOR inhibitor nanoparticle composition described herein, e.g., nab-sirolimus) which is by itself or combined with another composition effective for treating (such as delaying the progression of) cancer. In some embodiments, the cancer comprises one or more cells that express a KRAS mutant protein (e.g., a KRAS G12C mutant protein, a KRAS G12A mutant protein, a KRAS G12D mutant protein, a KRAS G12F mutant protein, a KRAS G12L mutant protein, a KRAS G12R mutant protein, a KRAS G12S mutant protein, a KRAS G12V mutant protein, a KRAS G13A mutant protein, a KRAS G13C mutant protein, a KRAS G13D mutant protein, a KRAS G13P mutant protein, a KRAS G13R mutant protein, a KRAS G13S mutant protein, a KRAS G13V mutant protein, a KRAS Q61E mutant protein, a KRAS Q61H mutant protein, a KRAS Q61K mutant protein, a KRAS Q61L mutant protein, a KRAS Q61P mutant protein, a KRAS Q61R mutant protein, a KRAS K117N mutant protein, a KRAS K117R mutant protein, a KRAS A146E mutant protein, a KRAS A146G mutant protein, a KRAS A146P mutant protein, a KRAS A146S mutant protein, a KRAS A146T mutant protein, or a KRAS A146V mutant protein). Additionally or alternatively, in some embodiments, the cancer comprises one or more cells that have at least one mTOR-activating aberration. In some embodiments, the cancer is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin. In some embodiments, the cancer is solid tumor, lung cancer, or bladder cancer. In some embodiments, the lung cancer is non-small cell lung cancer (NSCLC). The container may have a sterile access port (for example the container may be an intravenous solution bag or a vial having a stopper pierceable by a hypodermic injection needle). At least one agent in the composition is an mTOR inhibitor nanoparticle composition described herein, e.g., nab-sirolimus). In some embodiments, the label or package insert indicates that the composition is used in combination with a KRAS inhibitor (e.g., a KRAS G12C inhibitor, a KRAS G12A inhibitor, a KRAS G12D inhibitor, a KRAS G12F inhibitor, a KRAS G12L inhibitor, a KRAS G12R inhibitor, a KRAS G12S inhibitor, a KRAS G12V inhibitor, a KRAS G13A inhibitor, a KRAS G13C inhibitor, a KRAS G13D inhibitor, a KRAS G13P inhibitor, a KRAS G13R inhibitor, a KRAS G13S inhibitor, a KRAS G13V inhibitor, a KRAS Q61E inhibitor, a KRAS Q61H inhibitor, a KRAS Q61K inhibitor, a KRAS Q61L inhibitor, a KRAS Q61P inhibitor, a KRAS Q61R inhibitor, a KRAS K117N inhibitor, a KRAS K117R inhibitor, a KRAS A146E inhibitor, a KRAS A146G inhibitor, a KRAS A146P inhibitor, a KRAS A146S inhibitor, a KRAS A146T inhibitor, or a KRAS A146V inhibitor) for treating cancer (e.g., an exemplary cancer described herein). In some embodiments, the KRAS inhibitor is a KRAS G12C inhibitor, e.g., without limitation, isotorasib, which is also known as AMG 510 (Amgen/Beigene), MRTX849, which is also known as adagrasib (Mirati/Zai Lab), JAB-21822 (Jacobiopharma), GDC-6036 (Genentech), JDQ443 (Novartis), D-1553 (InventisBio and Merck Sharp & Dohme), GH35 (Genhouse Bio), GFH925 (GenFleet Therapeutics), BPI-421286 (Bettapharma), LY3537982, RMC-6291 (Revolution Medicine), RMC-8839 (Revolution Medicine), HBI-2438 (Huya Biosciences International, LLC), or JNJ-74699157 (Johnson & Johnson). In some embodiments, the KRAS inhibitor is a KRAS G12D inhibitor, e.g., without limitation, MRTX1133 (Mirati Therapeutics) or RMC-6236 (Revolution Medicines). In some embodiments, the KRAS inhibitor is a KRAS G12V inhibitor, e.g., without limitation, JAB-23000.

In some embodiments, the package insert provided with the article of manufacture or kit contains information about the indications, usage, dosage, administration, contraindications and/or warnings concerning the composition(s) provided with the article of manufacture or kit.

Moreover, the article of manufacture or kit may comprise (a) a first container with a composition contained therein, wherein the composition comprises an mTOR inhibitor nanoparticle composition described herein, e.g., nab-sirolimus), and (b) a second container with a composition contained therein, wherein the composition comprises KRAS inhibitor (e.g., a polypeptide, antibody, fusion polypeptide, antisense oligonucleotide or a small molecule drug that is capable of inhibiting the activity of a KRAS mutant protein described herein). In some embodiments, the second container contains a small molecule KRAS inhibitor (e.g., a KRAS inhibitor described herein). Additionally, the article of manufacture may further comprise an additional container comprising a pharmaceutically acceptable buffer, such as bacteriostatic water for injection (BWFI), phosphate-buffered saline, Ringer's solution, and dextrose solution. It may further include other materials desirable from a commercial and user standpoint, including other buffers, diluents, filters, needles, and syringes.

The kits provided herein are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

Those skilled in the art will recognize that several embodiments are possible within the scope and spirit of this invention. The invention will now be described in greater detail by reference to the following non limiting examples. The following examples further illustrate the invention but, of course, should not be construed as in any way limiting its scope.

All publications and patent applications cited in this specification are herein incorporated by reference as if each individual publication or patent application were specifically and individually indicated to be incorporated by reference.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Anti-Tumor Activity of Nab-Sirolimus in Combination with a KRAS G12C Inhibitor in Mice Bearing Human Non-Small Cell Lung Cancer (NSCLC) Tumor Xenografts

In this Example, the anti-tumor efficacies of each of (i) nab-sirolimus, (ii) everolimus (i.e., a sirolimus derivative), (iii) sotorasib (i.e., a small molecule KRAS G12C inhibitor), (iv) nab-sirolimus+sotorasib, and (v) everolimus+sotorasib were evaluated in mice bearing NCI-H2030 tumor xenografts. NCI-H2030 is a human non-small cell lung cancer (NSCLC) adenocarcinoma cell line harboring KRAS^{G12C} and STK11^{E317*} mutations. (NCI-H2030 mutation profile: KRAS^{G12C}, STK11^{E317*}, TP53^{G262V}.) The KRAS G12C mutation, which is common in NSCLC, leads to constitutive activation of the tumor growth-promoting RAS/MAPK signaling pathway. STK11 is a negative regulator of mTOR signaling. The mTOR pathway is often activated in patients with KRAS mutation and contributes to adaptive resistance to KRAS inhibitors.

Female athymic mice were implanted with human NCI-H2030 NSCLC cells in both flanks. When tumor volume reached approximately 100-150 mm³, animals were randomly divided into five groups (n=3/group), and administered with saline (control), a single agent drug, or a drug combination shown in Table A below according to the administration routes, dosages and administration frequencies shown in Table A1 below. Additional information about the treatment regimens in Table A1 are provided in Table A2.

TABLE A1
Treatment Groups for NCI-H2030 Tumor Xenograft Model

| | | | | | Route(s) of | |
|---|---|---|---|---|---|---|
| Group | #Mice | Tumor | Drug(s) | Dosage(s) | Administration | Frequency |
| 1 | 3 | NCI-H2030 | saline | Not | IV* | 10 ml |
| | | | (control) | applicable | | 2x weekly |
| 2 | 3 | NCI-H2030 | nab-sirolimus | 7.5 mg/kg | IV | 2x weekly |
| 3 | 3 | NCI-H2030 | everolimus | 3 mg/kg | PO** | qdx5 weekly† |
| 4 | 3 | NCI-H2030 | sotorasib | 30 mg/kg | PO | qdx5 weekly |
| 5 | 3 | NCI-H2030 | nab-sirolimus + | 7.5 mg/kg + | IV + | 2x weekly + |
| | | | sotorasib | 30 mg/kg | PO | qdx5 weekly |
| 6 | 3 | NCI-H2030 | everolimus + | 3 mg/kg + | PO + | qdx5 weekly + |
| | | | sotorasib | 30 mg/kg | PO | qdx5 weekly |

*IV = intravenous
**PO = per os (i.e., oral administration)
†daily, 5 times a week

TABLE A2
Additional Information about Treatment
Regimens in Tables A1, D, and F

| | | Weekly | % Clinical | |
|---|---|---|---|---|
| | | Dose | Weekly | |
| Material | Dose/Frequency^{a} | (mg/kg) | Dosing | Route |
| Saline | 10 mL/kg, twice | 0 | NA | IV |
| | weekly | | | |
| nab- | 7.5 mg/kg, twice | 15 | 45 | IV |
| Sirolimus | weekly | | | |
| Everolimus | 3 mg/kg, 5 days/week | 15 | 115 | PO |
| Sotorasib* | 30 mg/kg, 5 | 150 | 11 | PO |
| | days/week | | | |
| Adagrasib* | 30 mg/kg, 5 | 150 | 9 | PO |
| | days/week | | | |

^{a}Doses have been used in previous non-clinical studies. Dosing was once per day for 6 weeks; dosing regimen for each drug was consistent across models.
IV = intravenous
Nab = nanoparticle albumin-bound
PO = orally
*Martin et al. American Association for Cancer Research 2022 Annual Meeting. Poster 2670

As shown in FIG. 1A, the nab-sirolimus+sotorasib combination showed significantly greater tumor growth inhibition or “TGI” (109%) compared with single agent nab-sirolimus (68%, P=0.0102, ANOVA), everolimus (22%, P=0.0011) or sotorasib (20%, P=0.0002), and combination of everolimus and sotorasib (53%, P=0.0008) in the NCI-H2030 model. Table B shows that nab-sirolimus and sotorasib combination treatment was the only treatment that demonstrated a statistically significant difference in tumor growth inhibition (TGI), as compared to saline and to all other treatment groups (two-way ANOVA). No other treatment was found to demonstrate statistically significant TGI, as compared to saline or to other treatment groups.

TABLE B
Two-way ANOVA Analysis of TGI nab-Sirolimus +
Sotorasib vs. Other Treatment Groups

| Comparison of TGI | |
|---|---|
| (treatment group vs. treatment group) | p-value |
| nab-sirolimus + sotorasib vs. saline | 0.0003 |
| nab-sirolimus + sotorasib vs. nab-sirolimus | 0.0102 |
| nab-sirolimus + sotorasib vs. everolimus | 0.0011 |
| nab-sirolimus + sotorasib vs. sotorasib | 0.0002 |
| nab-sirolimus + sotorasib vs. everolimus + sotorasib | 0.0008 |

FIG. 1B provides waterfall plots showing tumor volume regression in in NCI-H2030-xeongrafted mice treated with saline, nab-sirolimus, everolimus, sotorasib, nab-sirolimus+sotorasib, and everolimus+sotorasib. The greater TGI observed in mice treated with nab-sirolimus+sotorasib correlated with significantly higher tumor response rate (tumor regression of more than −30% change in tumor volume) in the nab-sirolimus+sotorasib treatment group compared with all other treatment groups (P=0.0059, Chi-square). Table C shows the response rate (i.e., tumor regression>30%) among NCI-H2030-xenografted mice in treatment groups 1-6 from Table A. Tumor regression (e.g., >−30% change in tumor volume) was only observed in mice treated with nab-sirolimus+sotorasib. Combination treatment with everolimus+sotorasib failed to increase tumor response rate compared with single agent everolimus or single agent sotorasib.

TABLE C
Response Rate of NCI-H2030-Xenografted Mice (P = 0.0602)

| | | Response Rate % |
|---|---|---|
| Group | Drug(s) | (Tumor Regression >−30%) |
| 1 | saline (control) | 0 |
| 2 | nab-sirolimus | 0 |
| 3 | everolimus | 0 |
| 4 | sotorasib | 0 |
| 5 | nab-sirolimus + sotorasib | 50% |
| 6 | everolimus + sotorasib | 0 |

When comparing tumor growth curves, statistical significance was observed with nab-sirolimus+sotorasib vs single-agent nab-sirolimus (P=0.0001) or sotorasib (P<0.0001), and the combination of everolimus+sotorasib (P=0.0036).

All treatments were tolerable with no signs of toxicity and similar body weight change to saline control (see FIG. 1C).

Next, a similar set of experiments was performed in mice bearing human NCI-H2122 tumor xenografts. NCI-H2122 is a human NSCLC squamous cell cancer cell line that harbors KRAS^{G12C} and STK11 null (loss of function) mutations. (NCI-H2122 mutation profile: NCI-H2122: KRAS^{G12C}, STK11^{null}, TP53^{C176F}.) Briefly, female athymic mice were implanted with NCI-H2122 cells in in both flanks. When tumor volume reached approximately 100-150 mm³, animals were randomly divided into nine groups (n=5/group), and administered with saline (control), a single agent drug, or a drug combination shown in Table D below according to the administration routes, dosages and administration frequencies shown in Table D below. Adagrasib is a small molecule KRAS G12C inhibitor. Additional information about the treatment regimens in Table D are provided in Table A2.

TABLE D
Treatment Groups for NCI-H2122 Tumor Xenograft Model

| | | | | | Route(s) of | |
|---|---|---|---|---|---|---|
| Group | #Mice | Tumor | Drug(s) | Dosage(s) | Administration | Frequency |
| 1 | 5 | NCI-H2122 | saline | Not | IV* | 2x weekly |
| | | | (control) | applicable | | |
| 2 | 5 | NCI-H2122 | nab-sirolimus | 7.5 mg/kg | IV | 2x weekly |
| 3 | 5 | NCI-H2122 | everolimus | 3 mg/kg | PO** | qdx5 weekly† |
| 4 | 5 | NCI-H2122 | sotorasib | 30 mg/kg | PO | qdx5 weekly |
| 5 | 5 | NCI-H2122 | adagrasib | 30 mg/kg | PO | qdx5 weekly |
| 6 | 5 | NCI-H2122 | nab-sirolimus + | 7.5 mg/kg + | IV + | 2x weekly + |
| | | | sotorasib | 30 mg/kg | PO | qdx5 weekly |
| 7 | 5 | NCI-H2122 | everolimus + | 3 mg/kg + | PO + | qdx5 weekly + |
| | | | sotorasib | 30 mg/kg | PO | qdx5 weekly |
| 8 | 5 | NCI-H2122 | nab-sirolimus + | 7.5 mg/kg + | IV + | 2x weekly + |
| | | | adagrasib | 30 mg/kg | PO | qdx5 weekly |
| 9 | 5 | NCI-H2122 | everolimus + | 3 mg/kg + | PO + | qdx5 weekly + |
| | | | adagrasib | 30 mg/kg | PO | qdx5 weekly |

*IV = intravenous
**PO = per os (i.e., oral administration)
†daily, 5 times a week

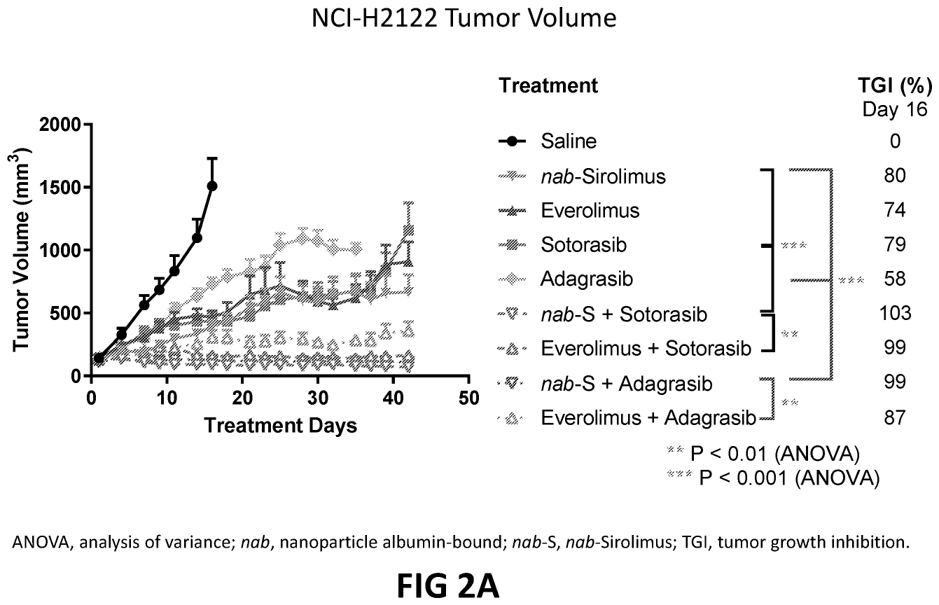

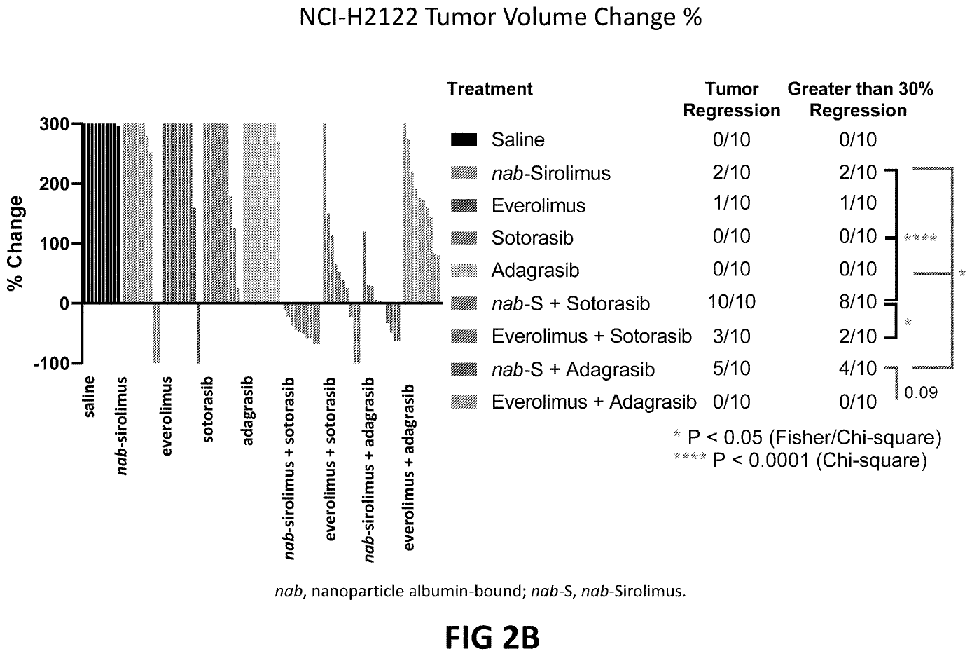

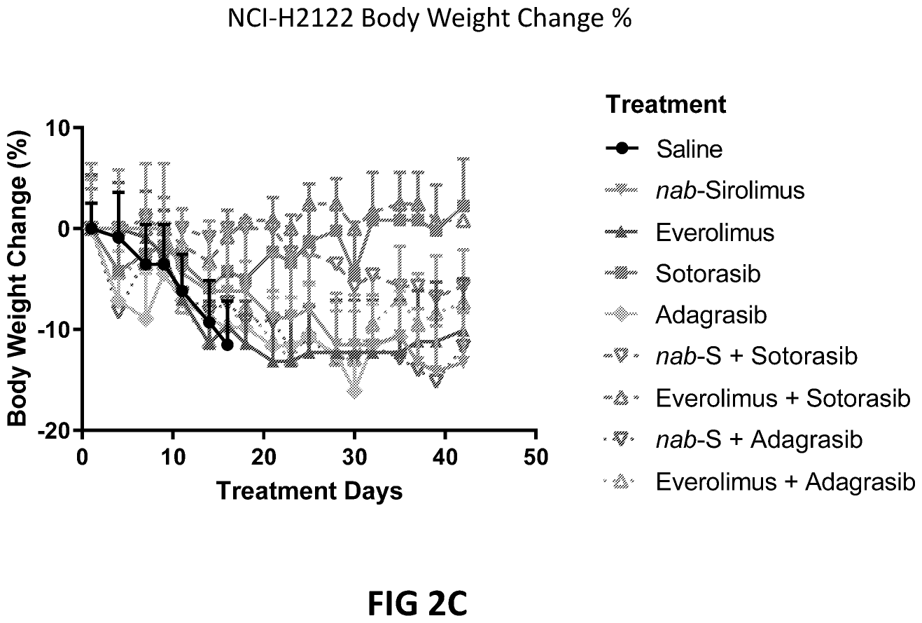

As shown in FIG. 2A, combination treatments with an mTOR inhibitor and a KRAS G12C inhibitor were significantly better in inhibiting the growth of NCI-H2122 tumors (TGI=103%), as compared to single agent treatment with nab-sirolimus (TGI=80%, P=0.0001, ANOVA) or single agent sotorasib (79%, P<0.0001). The nab-sirolimus+adagrasib combination showed significantly greater TGI (99%) compared with single agent nab-sirolimus (80%, P=0.0002), or single agent adagrasib (58%, P<0.0001). Single agent sotorasib was significantly more active in the NCI-H2122 model than adagrasib (P=0.0143). Unexpectedly, there was no significant difference in TGI of nab-sirolimus+sotorasib vs. nab-sirolimus vs. adagrasib. Further, nab-sirolimus+sotorasib and nab-sirolimus+adagrasib were statistically better in inhibiting tumor growth as compared to everolimus+sotorasib and everolimus+adagrasib, respectively: nab-sirolimus+sotorasib vs. everolimus+sotorasib: P=0.0036 (ANOVA); nab-sirolimus+adagrasib vs. everolimus+adagrasib: P=0.0013; nab-sirolimus+sotorasib vs. nab-sirolimus+adagrasib: P=not significant (ns).

FIG. 2B provides waterfall plots showing tumor volume regression in NCI-H2122-xeongrafted mice treated with saline, nab-sirolimus, everolimus, sotorasib, adagrasib, nab-sirolimus+sotorasib, everolimus+sotorasib, nab-sirolimus+adagrasib, and everolimus+adagrasib. The improved TGI in mice treated with nab-sirolimus+sotorasib or nab-sirolimus+adagrasib correlates with significantly higher tumor response rate (e.g., tumor regression more than −30% change in tumor volume) compared with saline and all single agent treatments (P<0.0001, Chi-square and P=0.0347, respectively). Tumor regression (e.g., more than −30% change in tumor volume) in the nab-sirolimus+sotorasib treatment group was 80%, whereas tumor regression in the everolimus+sotorasib treatment group was 20% (P=0.0230, Fisher's exact test). The combination of everolimus+sotorasib (FIG. 2B) failed to improve meaningful tumor regression rates over single-agent treatment with either agent. Tumor regression was numerically higher in the nab-sirolimus+adagrasib treatment group as compared to the everolimus+adagrasib treatment group (40% vs. 0%, respectively, P=0.0867). Table E shows the response rate (i.e., tumor regression>30%) among NCI-H2122-xenografted mice in treatment groups 1-9 from Table D. The response rates in mice treated with single agent nab-sirolimus or single agent everolimus were 10% and 20%, respectively, indicating that each single agent demonstrated minor antitumor activity. However, the response rates of mice treated with nab-sirolimus in combination with sotorasib or adagrasib (i.e., 80% and 40%, respectively) were unexpectedly found to be significantly higher than the response rates of mice treated with everolimus in combination with sotorasib or adagrasib (i.e., 20% and 0%, respectively).

TABLE E
Response Rate of NCI-H2122-Xenografted
Mice (P = not significant)

| | | Response Rate % |
|---|---|---|
| Group | Drug(s) | (Tumor Regression >−30%) |
| 1 | saline (control) | 0 |
| 2 | nab-sirolimus | 20% |
| 3 | everolimus | 10% |
| 4 | sotorasib | 0% |
| 5 | adagrasib | 0% |
| 6 | nab-sirolimus + sotorasib | 80% |
| 7 | everolimus + sotorasib | 20% |
| 8 | nab-sirolimus + adagrasib | 40% |
| 9 | everolimus + adagrasib | 0% |

All treatment groups demonstrated improved survival (i.e. days) as compared to saline control. The median survival time of mice given saline was 16 days. The median survival time of mice in other treatment groups had not been reached (p<0.0001, Log-rank test).

All treatments were tolerable with no signs of toxicity and similar body weight change to saline control (see FIG. 2C).

### Example 2: Anti-Tumor Activity of Nab-Sirolimus in Combination with a KRAS G12C Inhibitor in Mice Bearing Human Bladder Cancer Tumor Xenografts

In this Example, the anti-tumor efficacies of each of (i) nab-sirolimus, (ii) sotorasib, (iii) adagrasib, (iv) nab-sirolimus+sotorasib, and (v) nab-sirolimus+adagrasib were evaluated in mice bearing UMUC3 tumor xenografts. UMUC3 is a human transitional cell carcinoma (bladder cancer) cell line harboring KRAS^{G12C} and PTEN null mutations. PTEN is a negative regulator or mTOR activity (UMUC3 mutation profile: KRAS^{G12C}, PTEN^{null}, TP53^{F113C}, ATM^{Q2800fs}, CDKN2A^{null}, UGT2B17^{null}.)

Briefly, female athymic mice were implanted with NCI-H2122 cells in their right flanks. When tumor volume reached approximately 100-150 mm³, animals were randomly divided into nine groups (n=6/group), and administered with saline (control), a single agent drug, or a drug combination shown in Table F below according to the administration routes, dosages and administration frequencies shown in Table F below. Additional information about the treatment regimens in Table F are provided in Table A2.

TABLE F
Treatment Groups for UMUC3 Tumor Xenograft Model

| | | | | | Route(s) of | |
|---|---|---|---|---|---|---|
| Group | #Mice | Tumor | Drug(s) | Dosage(s) | Administration | Frequency |
| 1 | 6 | UMUC3 | saline | Not | IV* | 2x weekly |
| | | | (control) | applicable | | |
| 2 | 6 | UMUC3 | nab-sirolimus | 7.5 mg/kg | IV | 2x weekly |
| 3 | 6 | UMUC3 | sotorasib | 3 mg/kg | PO** | qdx5 weekly† |
| 4 | 6 | UMUC3 | adagrasib | 30 mg/kg | PO | qdx5 weekly |
| 5 | 6 | UMUC3 | nab-sirolimus + | 7.5 mg/kg + | IV + | 2x weekly + |
| | | | sotorasib | 30 mg/kg | PO | qdx5 weekly |
| 6 | 6 | UMUC3 | nab-sirolimus + | 7.5 mg/kg + | IV + | 2x weekly + |
| | | | adagrasib | 30 mg/kg | PO | qdx5 weekly |

*IV = intravenous
**PO = per os (i.e., oral administration)
†daily, 5 times a week

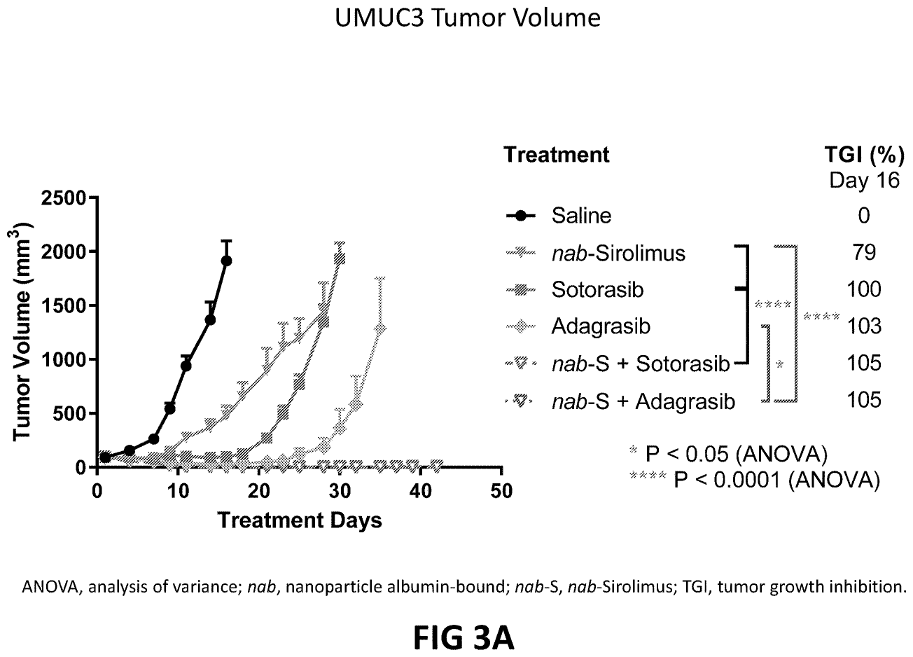

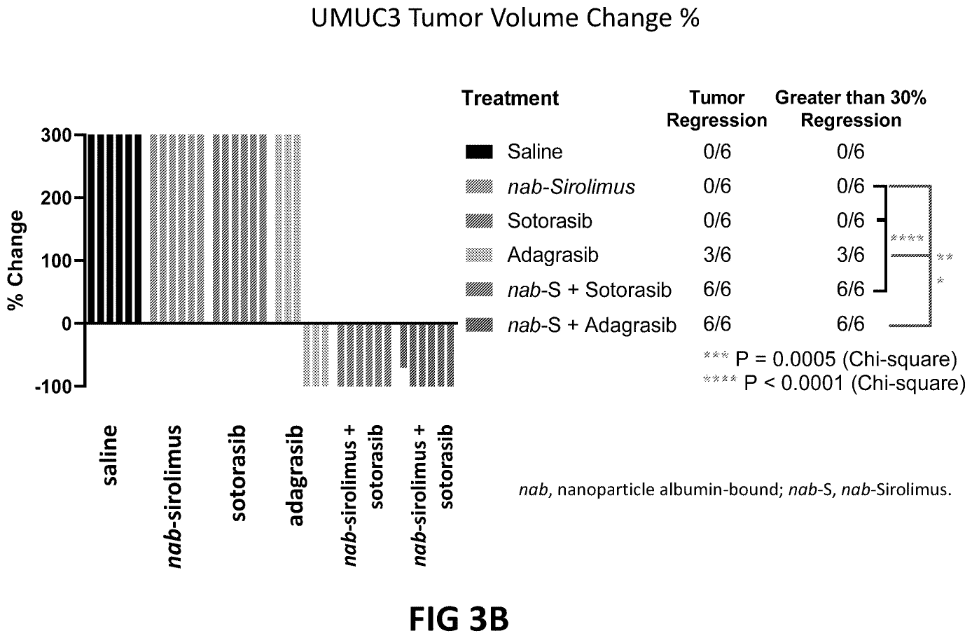

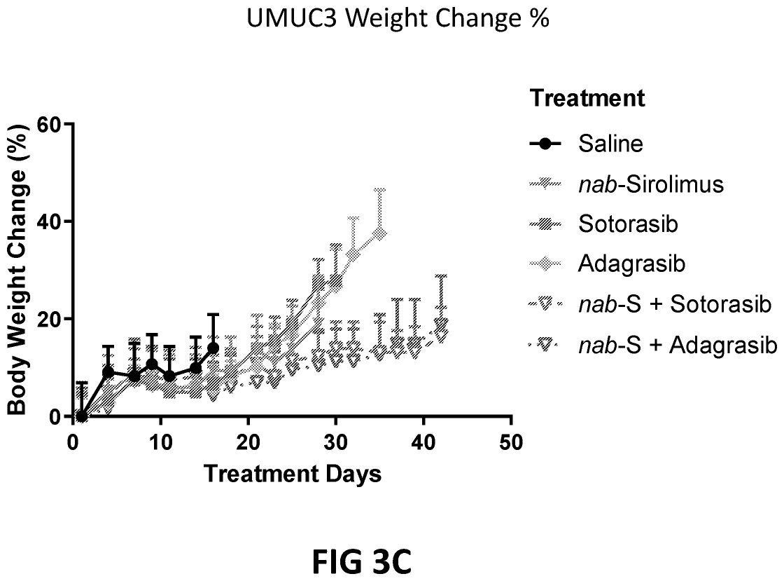

None of the mice (0/6) in any of the saline control group, the nab-sirolimus group, and the sotorasib group survived to the end of the study (i.e., Day 42). None of the mice (0/6) in any of these groups were tumor free. Among the mice treated with adagrasib, 1/6 survived to Day 42, and 3/6 were tumor-free during the study (median tumor free time: 24 days; range: 13-29 days). Among the mice treated with nab-sirolimus+sotorasib, 3/6 survived until Day 42, and 6/6 mice were tumor-free during the study (median tumor free time: 28 days; range: 25-34 days). Among the mice treated with nab-sirolimus+adagrasib, 3/6 survived until Day 42, and 5/6 were tumor free during study (median tumor free time: 31 days; range: 19-34 days). See FIG. 3A. Mice treated with nab-sirolimus+sotorasib or nab-sirolimus+adagrasib resulted in almost complete elimination of UMUC3 tumors (complete response, 6/6 of mice treated with nab-sirolimus+sotorasib demonstrated complete tumor regression, and 5/6 of mice treated with nab-sirolimus+adagrasib demonstrated complete tumor regression respectively). Combining nab-sirolimus with either KRAS inhibitor, i.e., sotorasib or adagrasib, showed significantly greater tumor growth suppression compared with single-agent nab-sirolimus, sotorasib, or adagrasib. See FIG. 3A. There was no significant difference in tumor growth suppression between combinations of nab-sirolimus with either sotorasib or adagrasib (see FIG. 3A).

Mice treated with nab-sirolimus+sotorasib showed significantly greater TGI (105%) compared to mice treated with single agent nab-sirolimus (79%, P<0.0001, ANOVA), or single agent sotorasib (100%, P<0.0001). Mice treated with nab-sirolimus+adagrasib showed significantly greater TGI (105%) compared to mice treated with single agent nab-sirolimus (79%, P<0.0001), or single agent adagrasib (103%, P=0.0374). In contrast the to the results from NCI-H2122 model (see FIG. 2A), single agent adagrasib was significantly more active in the UMUC3 model than sotorasib (P=0.0035). Unexpectedly, no significant difference in TGI was observed in mice treated with nab-sirolimus+sotorasib vs. mice treated with nab-sirolimus+adagrasib. No significant difference in TGI was observed in mice treated with nab-sirolimus+sotorasib vs. mice treated with nab-sirolimus+adagrasib.

The greater TGI observed in mice treated of nab-sirolimus+sotorasib or nab-sirolimus+adagrasib correlated with significantly higher rate of tumor response rate (e.g., tumor regression of more than −30% change in tumor volume) compared with saline and single agents (P<0.0001, Chi-square and P=0.0005, respectively). See FIG. 3B. Table G shows the response rate (i.e., tumor regression>30%) among UMUC3-xenografted mice in treatment groups 1-6 from Table F.

TABLE F
Response Rate of UMUC3-Xenografted Mice (P for nab-
sirolimus + sotorasib < 0.0001; P for
nab-sirolimus + adagrasib = 0.0005)

| | | Response Rate % |
|---|---|---|
| Group | Drug(s) | (Tumor Regression >−30%) |
| 1 | saline (control) | 0 |
| 2 | nab-sirolimus | 0% |
| 3 | sotorasib | 0% |
| 4 | adagrasib | 50% |
| 5 | nab-sirolimus + sotorasib | 100% |
| 6 | nab-sirolimus + adagrasib | 100% |

Mice treated with nab-sirolimus+sotorasib demonstrated improved survival as compared to mice treated with sotorasib alone (p=0.0183 (log rank)). Additionally, mice treated with nab-sirolimus+adagrasib demonstrated improved survival as compared to mice treated with adagrasib alone (p=0.0708 (log rank)).

All treatments were tolerable with no signs of toxicity and similar body weight change to saline control (see FIG. 3C).

### Example 3: Comparing the Anti-Tumor Activities of Single Agent Sotorasib Vs. Single Agent Adagrasib and of Nab-Sirolimus+Sotorasib Combination Therapy Vs. Nab-Sirolimus+Adagrasib Combination Therapy

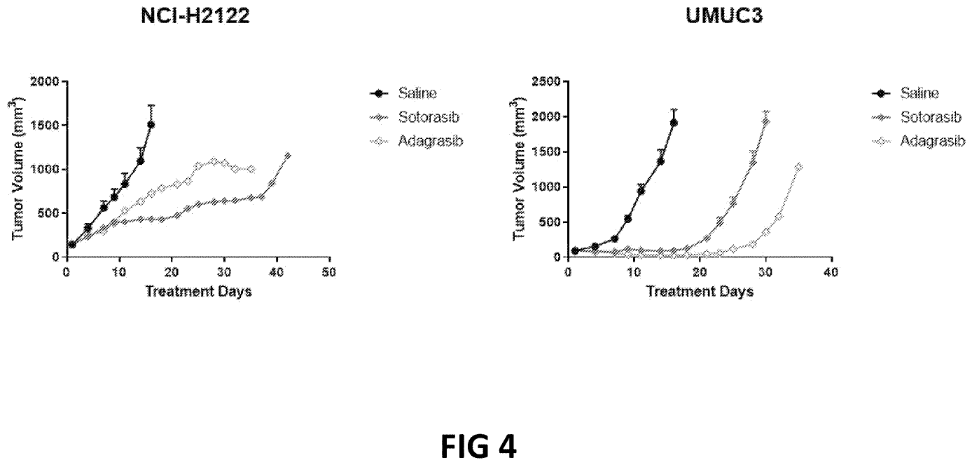

FIG. 4 shows the anti-tumor activity of single agent sotorasib and single agent adagrasib in mice bearing NCI-H2122 NSCLC tumors (left side, data taken from FIG. 2A), as well as the anti-tumor activity of single agent sotorasib and single agent adagrasib in mice bearing UMUC3 tumors (right side, data taken from FIG. 3A). Sotorasib was more effective than adagrasib in the NCI-H2122 model (P=0.0143), whereas adagrasib was more effective in the UMUC3 model (P=0.0035).

Without being bound by theory, the relative efficacy sotorasib and adagrasib may be tumor specific. Nevertheless, the combinations of nab-sirolimus+sotorasib and nab-sirolimus+adagrasib demonstrated significantly improved anti-tumor activity, as compared single agent nab-sirolimus, single agent sotorasib, and single agent adagrasib, in all the tumor models tested in Examples 1 and 2. See FIG. 1A, FIG. 2A, and FIG. 3A. Regardless of the relative anti-tumor activity of each single agent in the NSCLC and bladder cancer xenograft models, there was no difference in anti-tumor efficacy of nab-sirolimus+sotorasib and nab-sirolimus+adagrasib in either model. See e.g., FIG. 2A, and FIG. 3A.

By analyzing the rate of meaningful tumor response or meaningful tumor regression (e.g., tumor volume reduction of greater than 30%), treatment with nab-sirolimus+sotorasib and nab-sirolimus+adagrasib significantly increased the rate of meaningful tumor regression in all 3 models tested, not only when compared to treatment with single agent nab-sirolimus, single agent everolimus, single agent sotorasib, or single agent adagrasib, but also when compared to treatment with everolimus+sotorasib or everolimus+adagrasib. Combination treatments with everolimus+sotorasib or everolimus+adagrasib inhibited (such as slowed) tumor growth, but the everolimus+KRAS G12C inhibitor combination treatments did not result in any increase in tumor regression rate in any models tested.

In the H2030 model, the antitumor activity of single agent nab-sirolimus was numerically better than that of single agent everolimus (see FIG. 1A and FIG. 1B). By contrast, the antitumor activities of single agent nab-sirolimus and single agent everolimus were not significantly different in the H2122 model (see FIG. 2A and FIG. 2B). Surprisingly, response rates (e.g., tumor regression of >−30% in tumor volume) were significantly higher in mice treated with nab-sirolimus in combination with sotorasib or adagrasib than in mice treated with everolimus in combination with sotorasib or adagrasib.

In summary, nab-sirolimus+KRAS inhibitors resulted in partial responses (PR) and complete responses (CR), whereas everolimus+KRAS inhibitors resulted in some mice with stable disease (SD) and mice with disease progression.

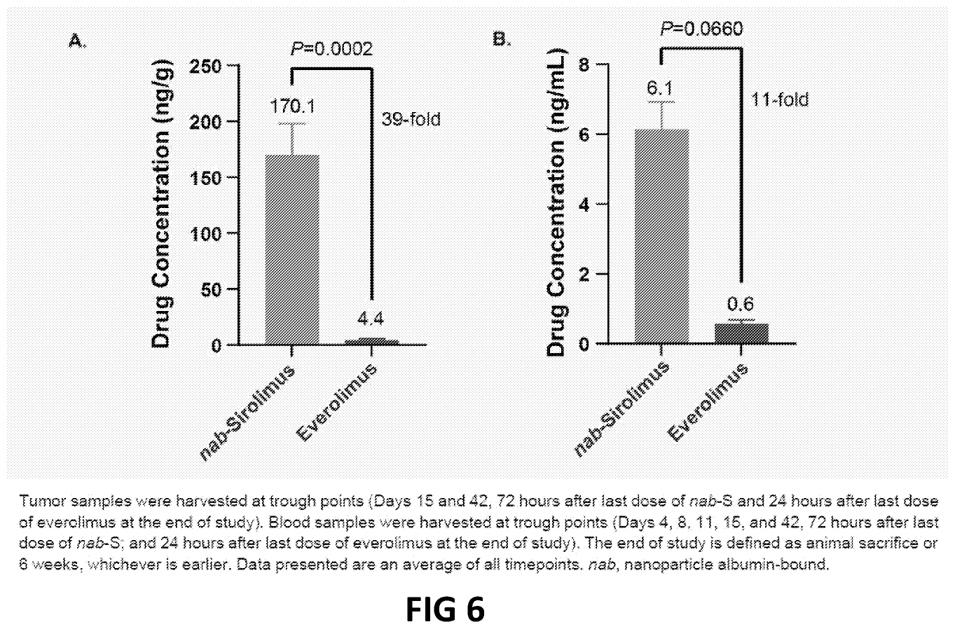

As shown in the Examples 1-3, nab-sirolimus, when combined with either sotorasib or adagrasib, showed synergistic antitumor activity with significantly greater suppression of tumor growth and meaningful tumor regressions compared to the single agents. This study confirms that consistent tumor growth inhibition and significantly higher tumor drug levels were observed with nab sirolimus than with everolimus. See e.g., FIG. 6. All treatments were tolerable with no overt signs of toxicity and produced a similar body weight change pattern when compared to the saline controls in each study. Results suggest that nab-sirolimus should be the preferred mTOR inhibitor for combination treatment with a KRAS inhibitor (e.g., adagrasib or sotorasib) in the clinic.

### Example 4: A Phase 1/2 Trial of a KRAS G12C Inhibitor in Combination with an mTOR Nanoparticle Composition in Patients with Advanced Solid Tumors with a KRAS G12C Mutation and Non-Small Cell Lung Cancer (NSCLC) with a KRAS G12C Mutation Overview

This Phase 1/2 study evaluates the safety and clinical activity of a KRAS G12C inhibitor (e.g., adagrasib or sotorasib) in combination with ABI-009 (i.e., an exemplary sirolimus/albumin nanoparticle composition also known as nab-sirolimus and FYARRO™) in cohorts of patients with advanced solid tumors/NSCLC with KRAS G12C mutation who have received prior therapy in the advanced or metastatic setting.

### Target Population

The target population is patients with advanced, unresectable, or metastatic solid tumor or NSCLC with KRAS G12C mutation.

### Number of Patients in Trial

The trial enrolls approximately 50-90 patients:
Phase 1: approximately 15-25 patients with solid tumors
Phase 2 cohorts:
   Cohort A: approximately 20-40 NSCLC patients with no prior KRAS G12C inhibitor exposure
   Cohort B: approximately 20-40 NSCLC patients with prior KRAS G12C inhibitor exposure

### (a) Objectives and Endpoints

### Phase 1—Objectives

The primary objectives of the Phase 1 portion of the trial include, but are not limited to (i) characterizing the safety and tolerability of a KRAS G12C inhibitor in combination with the mTOR nanoparticle composition ABI-009 (also known as FYARRO® and nab-sirolimus), in patients with advanced solid tumors harboring a KRAS G12C mutation and (ii) establishing the maximum tolerated dose (MTD) of the combination using one or more dosing regimens and/or to identify recommended Phase 2 combinatorial doses (RP2Ds) and regimens of the KRAS G12C inhibitor and ABI-009.

The secondary objectives of the Phase 1 portion of the trial include (i) evaluating the pharmacokinetics (PK) of the KRAS G12C inhibitor and ABI-009 when administered in combination and (ii) evaluating the clinical activity (e.g., clinical efficacy) of the KRAS G12C inhibitor in combination with ABI-009 in patients with solid tumor malignancies harboring a KRAS G12C mutation.

Exploratory objectives of the Phase 1 portion of the trial include, but are not limited to, (i) exploring potential pharmacodynamic markers of signal transduction inhibition in tumor tissue, (ii) evaluating the utility of detection of KRAS G12C mutations in plasma to identify the study population, and (iii) exploring correlations between tumor biomarkers, gene alterations, and efficacy.

### Phase 1—Endpoints

The primary endpoints of the Phase 1 portion of the trial include, but are not limited to, (i) safety, characterized by type, incidence, severity, timing, seriousness, relationship to study treatment of adverse events (AEs) and laboratory abnormalities, and number of patients modifying or discontinuing study treatment due to an adverse event (AE), from first dose of study treatment to 28 days after the last dose of study treatment and (ii) the maximum tolerated dose (MTD) and/or recommended Phase 2 dose (RP2D) for the KRAS G12C inhibitor and ABI-009 administered in combination. Safety parameters are used to assess number of patients with dose-limiting toxicity (DLT) to determine the MTD/RP2D.

The secondary endpoints of the Phase 1 portion of the trial include, but are not limited to, (i) plasma pharmacokinetic parameters for a the KRAS G12C inhibitor and ABI-009, (ii) objective response rate (ORR) as defined by defined by RECIST 1.1 (see Eisenhauer et al. (2009) Eur J. Cancer 45: 228-247), (iii) duration of response (DOR), (iv) progression-free survival (PFS), (v) PFS at 6 & 12 months, (vi) 1-Year Survival Rate, and (vii) overall survival (OS).

Overall survival (OS) is typically measured as the time from the start of treatment to date of death due to any cause. Progression free survival (PFS) is typically measured as the time from the start of treatment to the date of progressive disease (PD) or death due to any cause, whichever occurs first. Duration of response (DOR) is typically measured as the time from the start of treatment to the first documentation of objective tumor response (complete response (“CR”) or partial response (“PR”)) to the first documentation of either PD or death due to any cause, whichever occurs first. Objective response rate (ORR) is typically measured as the proportion of individuals (e.g., patients, subjects) that experience confirmed complete response (CR) or partial response (PR) based on RECIST v1.1 criteria during the time period from first dose of treatment until last dose of treatment. The exploratory endpoints of the Phase 1 portion of the trial include, but are not limited to, (i) level of KRAS G12C protein modification, (ii) dynamics of gene alterations in tumor tissue and circulating tumor DNA (ctDNA) and concordance between KRAS G12C mutation identified in tumor tissue versus ctDNA, (iii) mutations in RAS and other tumor genes potentially implicated in sensitivity and resistance to the KRAS G12C inhibitor in combination with ABI-009, including TSC1 & TSC2 mutations.

### Phase 2—Objectives

The primary objective of the Phase 2 portion of the trial includes, but is not limited to, evaluating the clinical efficacy of the KRAS G12C inhibitor in combination with ABI-009 in patients with NSCLC harboring a KRAS G12C mutation.

The secondary objectives of the Phase 2 portion of the trial include, but are not limited to, (i) evaluating the pharmacokinetics of the KRAS G12C inhibitor and ABI-009 when administered in combination, (ii) characterizing the safety and tolerability of the KRAS G12C inhibitor in combination with ABI-009 in patients with NSCLC harboring a KRAS G12C mutation, and (iii) evaluating the clinical activity of the KRAS G12C inhibitor in combination with ABI-009 in patients with solid tumor malignancies harboring a KRAS G12C mutation.

The exploratory objectives of the Phase 2 portion of the trial include, but are not limited to, (i) exploring potential pharmacodynamic markers of signal transduction inhibition in tumor tissue (ii) evaluating the utility of detection of KRAS G12C mutations in plasma to identify the study population, and (iii) exploring correlations between tumor biomarkers, gene alterations, and efficacy.

### Phase 2—Endpoints

The primary endpoint of the Phase 2 portion of the trial includes, but is not limited to, clinical efficacy based on ORR as defined by RECIST 1.1 (see Eisenhauer et al. (2009) Eur J Cancer 45: 228-247).

The secondary endpoints of the Phase 2 portion of the trial include, but are not limited to, (i) plasma pharmacokinetic concentrations for the KRAS G12C inhibitor and ABI-009, (ii) safety, characterized by type, incidence, severity, timing, seriousness, relationship to study treatment of AEs and laboratory abnormalities, and number of patients modifying or discontinuing study treatment due to an adverse event, from first dose of study treatment to 28 days after last dose of study treatment, (iii) duration of response (DOR), (iv) progression-free survival (PFS), (v) PFS at 6 & 12 months, (vi) 1-Year Survival Rate, and (vii) overall survival (OS). Overall survival (OS) is typically measured as the time from the start of treatment to date of death due to any cause. Progression free survival (PFS) is typically measured as the time from the start of treatment to the date of progressive disease (PD) or death due to any cause, whichever occurs first. Duration of response (DOR) is typically measured as the time from the start of treatment to the first documentation of objective tumor response (complete response (“CR”) or partial response (“PR”)) to the first documentation of either PD or death due to any cause, whichever occurs first. Objective response rate (ORR) is typically measured as the proportion of individuals (e.g., patients, subjects) that experience confirmed complete response (CR) or partial response (PR) based on RECIST v1.1 criteria during the time period from first dose of treatment until last dose of treatment.

The exploratory endpoints of the Phase 2 portion of the trial include, but are not limited to, (i) level of KRAS G12C protein modification, (ii) dynamics of gene alterations in tumor tissue and ctDNA, and concordance between KRAS G12C mutation identified in tumor tissue versus ctDNA, and (iii) mutations in RAS and other tumor genes potentially implicated in sensitivity and resistance to the KRAS G12C inhibitor in combination with ABI-009, including TSC1 & TSC2 mutations.

### (b) Study Design

This Example describes a Phase 1/2 evaluation of the safety, pharmacokinetics, and clinical activity of the combination of a KRAS G12C inhibitor with ABI-009 in patients with advanced solid tumor malignancies with KRAS G12C mutation and NSCLC with KRAS G12C mutation.

The study treatment (i.e., the KRAS G12C inhibitor in combination with ABI-009) is administered in 3-week cycles (or according to an alternative schedule). The KRAS G12C inhibitor is administered orally on an ongoing daily basis (or according to an alternative schedule). The KRAS G12C inhibitor is administered orally at a dose between 200 mg and 800 mg twice a day (i.e., bis en die or “BID”) or at a dose between 100 mg and 2000 mg once a day (i.e., “qd”). ABI-009 is administered via intravenous (IV) infusion on Days 1 and 8 every 21 days (i.e., twice every three weeks). Alternatively, the ABI-009 is administered once a week or once every three weeks. ABI-009 is administered at a dose between 1 mg/m² and 75 mg/m². Dosing may escalate or de-escalate dependent on toxicity experienced.

Patients receive study treatment at the discretion of the Investigator until disease progression, unacceptable adverse events, patient refusal, or death.

### Phase 1—Study Design

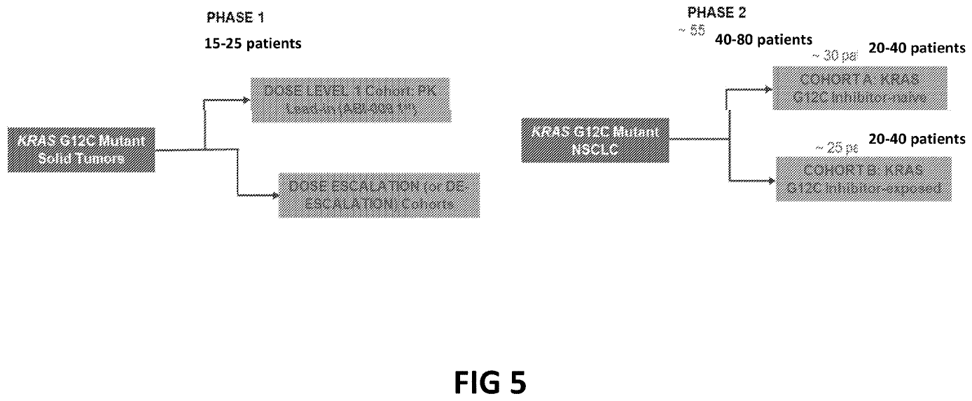

The study begins with a PK (pharmacokinetic) Lead-in at a first dose level of the KRAS G12C inhibitor and ABI-009 to evaluate the PK of both agents when given in combination. Approximately 24 patients are enrolled. The administration schedule for the PK Lead-in is provided in Table G1 and the administration schedule for Cycle 1 and all subsequent cycles is provided in Table G2 below, and schema for overall study is in FIG. 5.

TABLE G1

| | PK Lead-in | | | | | | | | | | | | | |
|---|---|---|---|---|---|---|---|---|---|---|---|---|---|---|
| Study Day | 1 | 2 | 3 | 4 | 5 | 6 | 7 | 8 | 9 | 10 | 11 | 12 | 13 | 14 |

| | | | | | | | | |
|---|---|---|---|---|---|---|---|---|
| ABI-009 Dosing | X | | | | | | | |
| KRAS G12C | | X | X | X | X | X | X | X |
| Inhibitor Dosing | | | | | | | | |

TABLE G2

| | Cycle 1 and Subsequent Cycles | | | | | | | | | | | | | | | | | | | | |
|---|---|---|---|---|---|---|---|---|---|---|---|---|---|---|---|---|---|---|---|---|---|
| Study Day | 1 | 2 | 3 | 4 | 5 | 6 | 7 | 8 | 9 | 10 | 11 | 12 | 13 | 14 | 15 | 16 | 17 | 18 | 19 | 20 | 21 |
| ABI-009 Dosing | X | | | | | | | X | | | | | | | | | | | | | |
| KRAS G12C | X | X | X | X | X | X | X | X | X | X | X | X | X | X | X | X | X | X | X | X | X |
| Inhibitor Dosing | | | | | | | | | | | | | | | | | | | | | |

For any specific regimen of the KRAS G12C inhibitor and ABI-009 administered, the MTD for the combination regimen is the dose associated with targeted toxicity rate of 0.30 during the first treatment cycle, with the acceptable toxicity probability interval of (0.25, 0.35).

### Phase 2—Study Design

After determination of the MTD and/or a potentially viable RP2D regimen, additional patients are enrolled into Phase 2 cohorts of up to a total of approximately 55 patients with NSCLC (approximately 30 patients in Cohort A and approximately 25 patients in Cohort B) to further evaluate the safety/tolerability and clinical activity.

If warranted, additional dose confirmation/expansion cohorts of patients are added. Objective Response Rate (ORR) in accordance with RECIST 1.1 (see Eisenhauer et al. (2009) Eur J Cancer. 45(2): 228-47) is the clinical activity endpoint for hypothesis testing. Patients experiencing clinical benefit in the judgment of the Investigator may continue study treatment beyond RECIST 1.1-defined disease progression. Patients discontinuing treatment are followed for receipt of subsequent anti-cancer therapies and survival.

### (c) Patient Selection and Enrollment Criteria

### Inclusion Criteria

The inclusion criteria for this Phase 1/2 clinical study are:
Histologically confirmed diagnosis:
   Phase 1: Histologically confirmed diagnosis of a solid tumor malignancy with KRAS G12C mutation in tumor tissue or plasma ctDNA.
   Phase 2: Histologically confirmed diagnosis of NSCLC with KRAS G12C mutation in tumor tissue or plasma ctDNA.
Unresectable or metastatic disease.
Not a candidate for definitive therapy (e.g., no available treatment with curative intent); in addition:
Patients in Phase 2 (Cohorts A and B) must have received prior therapy with platinum compound and checkpoint inhibitor (with any therapeutic intent).
Patients in Cohort A must have never received a prior KRAS G12C inhibitor.
Patients in Cohort B must have received a prior KRAS G12C inhibitor.
Presence of measurable disease per RECIST 1.1.
Age≥18 years.
Life expectancy of at least 3 months.
Most recent prior systemic therapy (e.g., chemotherapy, immunotherapy, or investigational agent) and radiation therapy discontinued at least 2 weeks before first dose of study treatment.
Recovery from the adverse effects of prior therapy at the time of enrollment to ≤Grade 1 (excluding alopecia, peripheral neuropathy, and parameters superseded by other eligibility criteria, such as hematology parameters).
Eastern Cooperative Oncology Group (ECOG) performance status of 0 or 1 (see, e.g., www(dot)ecog-acrin(dot)org/resources/ecog-performance-status).
   Adequate organ function, as determined by laboratory values within the screening period:
   Absolute neutrophil count≥1,500/mm³ (≥1.5×10⁹/L);
   Platelet count≥100,000/mm³ (100×10⁹/L);
   Hemoglobin≥9 g/dL, in the absence of transfusions for at least 2 weeks;
   Total bilirubin≤1.5×Upper Limit of Normal (ULN) (if associated with liver metastases or Gilbert's disease, ≤3×ULN);
   Aspartate transaminase (AST) and alanine transaminase (ALT)≤3.0×ULN (if associated with liver metastases, ≤5×ULN)
   Creatinine clearance≥50 mL/min or glomerular filtration rate≥50 mL/min/1.73 m² calculated using a validated prediction equation (e.g., Cockcroft-Gault, Modification of Diet in Renal Disease (MDRD), or 24-hour urine CrCl).
Women of childbearing potential (WOCBP) or men whose partner is a WOCBP agree to use contraception while participating in this study, and for a period of 6 months following termination of study treatment.

### Exclusion Criteria

Active brain metastases or carcinomatous meningitis. Patients with brain metastases are eligible if:
   Brain metastases are adequately treated and
   Patients are neurologically stable for at least 2 weeks prior to enrollment and
   Steroid dosing≤10 mg daily prednisone (or equivalent).
History of significant hemoptysis or hemorrhage within 4 weeks of the first dose of study treatment.
Major surgery within 4 weeks of first dose of study treatment.
History of intestinal disease, inflammatory bowel disease, major gastric surgery, or other gastrointestinal conditions (e.g., uncontrolled nausea, vomiting, malabsorption syndrome) likely to alter absorption of study treatment or result in inability to swallow oral medications.
Any of the following cardiac abnormalities within the last 6 months prior to enrollment:
   Unstable angina pectoris or myocardial infarction;
   Congestive heart failure≥New Your Heart Association Class 3;
   Prolonged corrected QT (QTc)>480 milliseconds on ECG during screening period or medical or family history of congenital Long QT Syndrome;
   Symptomatic or uncontrolled atrial fibrillation or other clinically significant arrhythmia.
History of stroke or transient ischemic attack within the previous 6 months.
Ongoing need for a medication with a known risk of Torsades de Pointes or substrate of CYP3A with narrow therapeutic index; strong inhibitor or inducer of CYP3A and/or P-gp; strong inhibitor of breast cancer resistant protein (BCRP); and proton pump inhibitors that cannot be switched to alternative treatment prior to study entry. At least 5 half-lives must have elapsed since discontinuation of these concomitant medications prior to starting study treatment.
Known or suspected presence of another malignancy that could be mistaken for the malignancy under study during disease assessments.
Known human immunodeficiency virus (HIV) infection or acute or chronic hepatitis B or C infection. Patients treated for hepatitis C with no detectable viral load and patients treated for HIV with no detectable viral load for at least 1 month are permitted.
Immunocompromised patients (solid organ or bone marrow transplant patients on current immunosuppression, congenital immunodeficiency, or immunosuppressive medications putting the patient at risk for opportunistic infection in the opinion of the investigator).
Live vaccine within 14 days of first dose of study treatment
History of interstitial lung disease or radiation pneumonitis requiring steroid treatment, or any evidence of clinically active interstitial lung disease or pneumonitis.
Uncontrolled diabetes within 4 weeks of study treatment.
Known hypersensitivity to a KRAS G12C inhibitor or ABI-009, or to any excipient.
Pregnancy (WOCBP must have a negative serum or urine pregnancy test documented within the screening period prior to start of study drug).
Breastfeeding or planning to breast feed during the study or within 6 months after study treatment.
Any serious illness, uncontrolled inter-current illness, psychiatric illness, active or uncontrolled infection, or other medical history, including laboratory results, which, in the Investigator's opinion, would be likely to interfere with the patient's participation in the study, or with the interpretation of the results.

### Dietary Restrictions

Patients are to avoid the following substances due to the possibility of interactions of these substances with the pharmacokinetics of the KRAS G12C inhibitor and/or ABI-009:
Grapefruit juice;
Curcumin/turmeric; and
St. John's wort and other herbal preparations.

### (d) Patient Treatment

The combination treatment (i.e., the KRAS G12C inhibitor and ABI-009) is administered in 21-day cycles. For the PK lead-in cycle only, the KRAS G12C inhibitor is administered orally starting on Day 8, and ABI-009 is administered intravenously on Day 1 over 30 minutes. For all other cycles, the KRAS G12C inhibitor is administered orally starting on Day 1, and ABI 009 is administered intravenously on Days 1 and 8 over 30 minutes. The KRAS G12C inhibitor is administered prior to ABI-009. The twice daily dosing of the KRAS G12C inhibitor is at 12-hour intervals to the extent possible. ABI-009 is dosed based on body surface area. Patients are dosed based on their height at beginning of study and their weight at beginning of study, and the dose is not adjusted unless a patient's body weight changes by more than 5% from baseline.

### Phase 1 Segment

The study begins with evaluation of the KRAS G12C inhibitor administered at a dose between 200 mg and 800 mg BID (i.e., twice a day) in combination with ABI-009 at a dose between 1 mg/m² and 75 mg/m². The combination regimen is administered in in 21-day cycles. The regimens of the KRAS G12C inhibitor and/or ABI-009 are adapted based on the observed toxicity, toxicity resolution and/or PK profiles. Other dosages of the KRAS G12C inhibitor and/or ABI-009 may be explored depending on emerging data. Allowance is made for dose reduction of both the KRAS G12C inhibitor and ABI-009 as needed to manage adverse events (AEs) and in pursuit of the RP2D regimen.

### Regimens Viable for Phase 2

Regimens of the KRAS G12C inhibitor in combination with ABI-009 considered viable for use in Phase 2 are typified by sufficient tolerability to anticipate delivery of at least 80% of the intended dose intensity during at least 2 treatment cycles and must be at or below the maximum tolerated dose (defined as the dose associated with a targeted toxicity rate of 0.30 during the first treatment cycle, with the acceptable toxicity probability interval of (0.25, 0.35)).

### Phase 2 Dose Confirmation/Expansion Segment

The Phase 2 segment of the study evaluates the clinical efficacy of the KRAS G12C inhibitor in combination with ABI-009 in cohorts of patients having NSCLC with KRAS G12C mutation and specified tumor histology, treatment history, and baseline characteristics (see Patient Selection and Enrollment Criteria). Patients receive treatment with the KRAS G12C inhibitor and ABI-009 using the dose levels and regimen determined in the dose escalation part of the study.

### (e) Efficacy Endpoint Definitions and Analyses Objective Response Rate (ORR)

Objective disease response is categorized in accordance with RECIST 1.1 criteria (see Eisenhauer et al. (2009) Eur J Cancer. 45(2): 228-47). Objective Response Rate is determined as the percent of patients documented to have a confirmed complete response (CR) or partial response (PR).

### Duration of Response (DOR)

Duration of Response is defined as the time from date of the first documentation of objective tumor response (CR or PR) to the first documentation of Objective Progression of Disease (PD) or to death due to any cause in the absence of documented PD. The Kaplan Meier method is used for the subgroup of patients with an objective response in order to obtain the estimate of median DOR.

### Progression-Free Survival (PFS)

Progression-free survival is determined as the time from date of first study treatment to first PD or death due to any cause in the absence of documented PD. The Kaplan-Meier method is used to obtain the estimate of median PFS time.

### Overall Survival (OS)

Time to death is determined as the time from date of first study treatment to death due to any cause. The Kaplan-Meier is used to estimate the median OS and 1-year Survival Rate; the 95% confidence interval of the 1-year survival rate is reported.

### Subgroup Analyses

Baseline characteristics in Phase 2 evaluated in subgroup analyses include gender, age, smoking status, and tumor DNA source for detection of KRAS G12C mutation (e.g., tumor tissue or ctDNA).

### Pharmacokinetic Analyses

Pharmacokinetic parameters are determined for the PK-evaluable population using standard non-compartmental methods. PK parameters include, but are not limited to, the following:
Cmax (ng/mL): Observed maximum plasma concentration of the KRAS G12C inhibitor and/or ABI-009 during a dosing interval
Cmin (ng/mL): Minimum observed concentration of the KRAS G12C inhibitor and/or ABI-009 during a dosing interval
tmax (hr): Observed time to maximum plasma concentration of the KRAS G12C inhibitor and/or ABI-009 during a dosing interval
t1/2 (hr): Terminal elimination half-life of the KRAS G12C inhibitor and/or ABI-009
AUClast (ng*h/mL): Area under the plasma concentration-time curve of the KRAS G12C inhibitor and/or ABI-009 from time zero to the last measurable time point calculated by log-linear trapezoidal summation
AUC∞ (ng*h/mL): Area under the plasma concentration-time curve of the KRAS G12C inhibitor and/or ABI-009 from time zero to infinity, calculated by log-linear trapezoidal summation and extrapolated to infinity by addition of the last observed quantifiable plasma concentration divided by the elimination rate constant λz (if % AUCext>20%, AUCO-∞ is not reported)
CL/F (L/hr): Apparent clearance of the KRAS G12C inhibitor after oral administration
Vz/F (L): Apparent volume of distribution of the KRAS G12C inhibitor after oral administration
CL (L/hr): Apparent total body clearance of the KRAS G12C inhibitor and/or ABI-009 from plasma.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 5

Upon analysis of colon cancer patients treated with nab-rapamycin, it was found that patients with KRAS wildtype responded better than patients with a KRAS aberration (44% vs. 20% overall response rate). The results indicate that mutations in KRAS pathway are contributing to drug resistance against nab-sirolimus in cancer patients with mTOR pathway activation. Accordingly, combination strategies with KRAS/NRAS inhibitors for patients with these mutations may be warranted.

### Example 6

### Method

Mice bearing NCI-H2122 tumors were treated for 6 weeks or until tumors exceeded 2000 mm³ in size. At the end of study, tumors were harvested and analyzed for biomarkers by western blot.

### Result

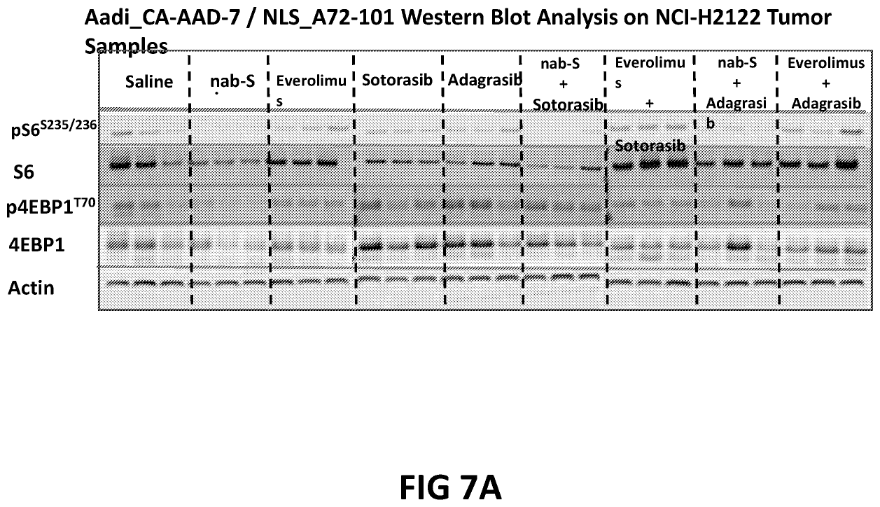

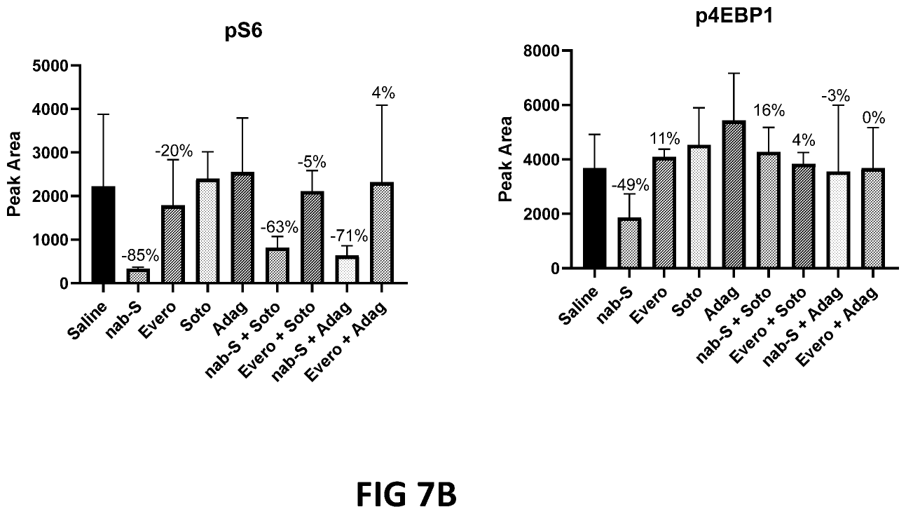

Following prolonged treatment, nab-sirolimus alone or in combination with sotorasib or adagrasib showed stronger inhibition of mTORC1 target phospho-S6 compared with the corresponding everolimus alone or combination groups. See FIGS. 7A-7B. In addition, nab-sirolimus alone also showed stronger inhibition of mTORC1 target phospho-4EBP1 compared with everolimus alone. The results demonstrated that nab-sirolimus strongly inhibited mTORC1 activity alone or in combination with KRAS inhibitors.

1. A method of treating cancer in an individual, comprising administering to the individual:
(a) an effective amount of a composition comprising nanoparticles comprising an mTOR inhibitor and an albumin, and
(b) an effective amount of a KRAS inhibitor.

2. The method of claim 1, wherein the mTOR inhibitor is a limus drug.

3. The method of claim 2, wherein the limus drug is sirolimus.

4. The method of any one of claims 1-3, wherein the average diameter of the nanoparticles in the composition is no greater than about 150 nm.

5. The method of claim 4, wherein the average diameter of the nanoparticles in the composition is no greater than about 120 nm.

6. The method of any one of claims 1-5, wherein the weight ratio of the albumin to the mTOR inhibitor in the nanoparticle composition is no greater than about 10:1.

7. The method of any one of claims 1-6, wherein the nanoparticles comprise the mTOR inhibitor associated with the albumin.

8. The method of claim 7, wherein the nanoparticles comprise the mTOR inhibitor coated with the albumin.

9. The method of any one of claims 1-8, wherein the mTOR inhibitor nanoparticle composition is administered intravenously or subcutaneously.

10. The method of claim 9, wherein the mTOR inhibitor nanoparticle composition is administered intravenously.

11. The method of any one of claims 1-10, wherein the KRAS inhibitor is an antibody, a peptide, a protein, an antisense oligonucleotide, or a small molecule that inhibits the activity of the KRAS mutant protein.

12. The method of claim 11, wherein the KRAS inhibitor is a small molecule.

13. The method of claim 12, wherein the KRAS inhibitor is a small molecule KRAS G12C inhibitor selected from the group consisting of: sotorasib, adagrasib, JAB-21822, GDC-6036, JDQ443, D-1553, GH35, GFH925, BPI-421286, and LY3537982, RMC-6291, RMC-8839, HBI-2438, and JNJ-74699157.

14. The method of claim 13, wherein the KRAS G12C inhibitor small molecule is sotorasib or adagrasib.

15. The method of claim 14, wherein the sotorasib or the adagrasib is administered orally.

16. The method of any one of claims 1-15, wherein the cancer comprises one or more cancer cells that express a KRAS G12C mutant protein.

17. The method of claim 12, wherein the KRAS inhibitor is a small molecule KRAS G12D inhibitor selected from the group consisting of: MRTX1133 and RMC-6236.

18. The method of any one of claims 1-11 and 17, wherein the cancer comprises one or more cancer cells that express a KRAS G12D mutant protein.

19. The method of claim 12, wherein the KRAS inhibitor is a small molecule KRAS G12V inhibitor, and wherein the small molecule KRAS G12V inhibitor is JAB-23000.

20. The method of any one of claims 1-11 and 19, wherein the cancer comprises one or more cancer cells that express a KRAS G12V mutant protein.

21. The method of any one of claims 1-20, comprises one or more cancer cells that express a KRAS mutant protein and/or have at least one mTOR-activating aberration

22. The method of any one of claims 1-21, wherein the cancer that comprises one or more cancer cells that express a KRAS mutant protein and/or have at least one mTOR-activating aberration is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin.

23. The method of claim 22, wherein the cancer is solid tumor, lung cancer, bladder cancer, appendiceal cancer, colorectal cancer, small bowel cancer, pancreatic cancer, or tumor of unknown origin.

24. The method of claim 23, wherein the cancer is solid tumor, lung cancer or bladder cancer.

25. The method of claim 23, wherein the solid tumor is advanced, unresectable, and/or metastatic solid tumor.

26. The method of claim 24, wherein the lung cancer is non-small cell lung cancer (NSCLC).

27. The method of claim 26, wherein the NSCLC is advanced, unresectable, and/or metastatic NSCLC.

28. The method of any one of claims 1-27, wherein the mTOR inhibitor nanoparticle composition and the KRAS inhibitor are administered simultaneously.

29. The method of any one of claims 1-27, wherein the mTOR inhibitor nanoparticle composition and the KRAS inhibitor are administered concurrently.

30. The method of any one of claims 1-27, wherein the mTOR inhibitor nanoparticle composition and the KRAS inhibitor are administered sequentially.

31. The method of claim 30, wherein the mTOR inhibitor nanoparticle composition is administered weekly, once every three weeks, or twice every three weeks.

32. The method of claim 31, wherein the KRAS inhibitor is administered daily or twice every day.

33. The method of any one of claims 1-32, wherein the individual is human.

34. The method of any one of claims 1-33, wherein the method comprises selecting the individual for treatment based on the presence of one or more cancer cells with at least one mTOR-activating aberration prior to the administration of the mTOR inhibitor nanoparticle composition and the KRAS inhibitor.

35. The method of claim 34, wherein the mTOR-activating aberration comprises a mutation in an mTOR-associated gene.

36. The method of claim 34 or 35, wherein the mTOR-activating aberration is in at least one mTOR-associated gene selected from the group consisting of: AKT1, FLT-3, MTOR, PIK3CA, PIK3CG, TSC1, TSC2, RHEB, STK11, NF1, NF2, TP53, FGFR4, BAP1, KRAS, NRAS, NRF2, KEAP1, and PTEN.

37. The method of claim 36, wherein the mTOR activating aberration is in TSC1 and/or TSC2.

38. The method of any one of claims 1-37, wherein the method comprises selecting the individual for treatment based on the presence of one or more cancer cells that express a KRAS mutant protein.

39. The method of claim 38, wherein the KRAS mutant protein is a KRAS G12C mutant protein, a KRAS GT2D mutant protein, or a KRAS GT2V mutant protein.

40. A kit for treating cancer in a subject comprising:
(a) a composition comprising nanoparticles comprising an mTOR inhibitor and an albumin, and
(b) instructions for administering an effective amount of the mTOR inhibitor nanoparticle composition and an effective amount of a KRAS inhibitor to a subject who has a cancer that comprises one or more cancer cells that express a KRAS mutant protein and/or have at least one mTOR-activating aberration.

41. The kit of claim 40, wherein the mTOR inhibitor is a limus drug.

42. The kit of claim 40 or 41, wherein the limus drug is sirolimus.

43. The kit of any one of claims 40-42, wherein the average diameter of the nanoparticles in the composition is no greater than about 150 nm.

44. The kit of claim 43, wherein the average diameter of the nanoparticles in the composition is no greater than about 120 nm.

45. The kit of any one of claims 40-44, wherein the weight ratio of the albumin to the mTOR inhibitor in the nanoparticle composition is no greater than about 10:1.

46. The kit of any one of claims 40-45, wherein the nanoparticles comprise the mTOR inhibitor associated with the albumin.

47. The kit of claim 46, wherein the nanoparticles comprise the mTOR inhibitor coated with the albumin.

48. The kit of any one of claims 40-47, wherein the KRAS inhibitor is an antibody, a peptide, a protein, an antisense oligonucleotide, or a small molecule that inhibits the activity of the KRAS mutant protein.

49. The kit of claim 48, wherein the KRAS inhibitor is a small molecule.

50. The kit of claim 49, wherein the KRAS inhibitor is a small molecule KRAS G12C inhibitor selected from the group consisting of sotorasib, adagrasib, JAB-21822, GDC-6036, JDQ443, D-1553, GH35, GFH925, BPI-421286, and LY3537982, RMC-6291, RMC-8839, HBI-2438, and JNJ-74699157.

51. The kit of claim 50, wherein the KRAS inhibitor is a small molecule KRAS G12D inhibitor selected from the group consisting of: MRTX1133 and RMC-6236.

52. The kit of claim 51, wherein the KRAS inhibitor is a small molecule KRAS G12V inhibitor, and wherein the small molecule KRAS G12V inhibitor is JAB-23000.

53. The kit of any one of claims 40-52, wherein the cancer is solid tumor, lung cancer, bladder cancer, appendiceal cancer, colorectal cancer, small bowel cancer, pancreatic cancer, uterine cancer, endometrial cancer, cervical cancer, testicular cancer, cholangiocarcinoma, myelodysplastic cancer, or tumor of unknown origin.

54. The kit of any one of claims 40-53, wherein the individual is a human.
